SEQUENCE LISTING (2 sequences)

SEQ ID NO: 1 - protein, 2944 aa, Homo sapiens
MTLRLLVAALCAGILAEAPRVRAQHRERVTCTRLYAADIVFLLDGSSSIGRSNFREVRSFLEGLVLPFSGAASAQGVRFATVQYSDDPRTEFGLDALGSGGDVIRAIRELSYKGGNTRTGAAILHVADHVFLPQLARPGVPKVCILITDGKSQDLVDTAAQRLKGQGVKLFAVGIKNADPEELKRVASQPTSDFFFFVNDFSILRTLLPLVSRRVCTTAGGVPVTRPPDDSTSAPRDLVLSEPSSQSLRVQWTAASGPVTGYKVQYTPLTGLGQPLPSERQEVNVPAGETSVRLRGLRPLTEYQVTVIALYANSIGEAVSGTARTTALEGPELTIQNTTAHSLLVAWRSVPGATGYRVTWRVLSGGPTQQQELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATELPGQRVRVSWSPVPGATQYRIIVRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQKGEPGEMGLRGQVGPPGDPGLPGRTGAPGPQGPPGSATAKGERGFPGADGRPGSPGRAGNPGTPGAPGLKGSPGLPGPRGDPGERGPRGPKGEPGAPGQVIGGEGPGLPGRKGDPGPSGPPGPRGPLGDPGPRGPPGLPGTAMKGDKGDRGERGPPGPGEGGIAPGEPGLPGLPGSPGPQGPVGPPGKKGEKGDSEDGAPGLPGQPGSPGEQGPRGPPGAIGPKGDRGFPGPLGEAGEKGERGPPGPAGSRGLPGVAGRPGAKGPEGPPGPTGRQGEKGEPGRPGDPAVVGPAVAGPKGEKGDVGPAGPRGATGVQGERGPPGLVLPGDPGPKGDPGDRGPIGLTGRAGPPGDSGPPGEKGDPGRPGPPGPVGPRGRDGEVGEKGDEGPPGDPGLPGKAGERGLRGAPGVRGPVGEKGDQGDPGEDGRNGSPGSSGPKGDRGEPGPPGPPGRLVDTGPGAREKGEPGDRGQEGPRGPKGDPGLPGAPGERGIEGFRGPPGPQGDPGVRGPAGEKGDRGPPGLDGRSGLDGKPGAAGPSGPNGAAGKAGDPGRDGLPGLRGEQGLPGPSGPPGLPGKPGEDGKPGLNGKNGEPGDPGEDGRKGEKGDSGASGREGRDGPKGERGAPGILGPQGPPGLPGPVGPPGQGFPGVPGGTGPKGDRGETGSKGEQGLPGERGLRGEPGSVPNVDRLLETAGIKASALREIVETWDESSGSFLPVPERRRGPKGDSGEQGPPGKEGPIGFPGERGLKGDRGDPGPQGPPGLALGERGPPGPSGLAGEPGKPGIPGLPGRAGGVGEAGRPGERGERGEKGERGEQGRDGPPGLPGTPGPPGPPGPKVSVDEPGPGLSGEQGPPGLKGAKGEPGSNGDQGPKGDRGVPGIKGDRGEPGPRGQDGNPGLPGERGMAGPEGKPGLQGPRGPPGPVGGHGDPGPPGAPGLAGPAGPQGPSGLKGEPGETGPPGRGLTGPTGAVGLPGPPGPSGLVGPQGSPGLPGQVGETGKPGAPGRDGASGKDGDRGSPGVPGSPGLPGPVGPKGEPGPTGAPGQAVVGLPGAKGEKGAPGGLAGDLVGEPGAKGDRGLPGPRGEKGEAGRAGEPGDPGEDGQKGAPGPKGFKGDPGVGVPGSPGPPGPPGVKGDLGLPGLPGAPGVVGFPGQTGPRGEMGQPGPSGERGLAGPPGREGIPGPLGPPGPPGSVGPPGASGLKGDKGDPGVGLPGPRGERGEPGIRGEDGRPGQEGPRGLTGPPGSRGERGEKGDVGSAGLKGDKGDSAVILGPPGPRGAKGDMGERGPRGLDGDKGPRGDNGDPGDKGSKGEPGDKGSAGLPGLRGLLGPQGQPGAAGIPGDPGSPGKDGVPGIRGEKGDVGFMGPRGLKGERGVKGACGLDGEKGDKGEAGPPGRPGLAGHKGEMGEPGVPGQSGAPGKEGLIGPKGDRGFDGQPGPKGDQGEKGERGTPGIGGFPGPSGNDGSAGPPGPPGSVGPRGPEGLQGQKGERGPPGERVVGAPGVPGAPGERGEQGRPGPAGPRGEKGEAALTEDDIRGFVRQEMSQHCACQGQFIASGSRPLPSYAADTAGSQLHAVPVLRVSHAEEEERVPPEDDEYSEYSEYSVEEYQDPEAPWDSDDPCSLPLDEGSCTAYTLRWYHRAVTGSTEACHPFVYGGCGGNANRFGTREACERRCPPRVVQSQGTGTAQD

SEQ ID NO: 2 - DNA, 9231 bp, Homo sapiens
  source 1..9231: mol_type = other DNA
gacttttctctgctctgcccgagagactgcagcggcggcggcgggagcgggcggacgcgcaggatgacgctgcggcttctggtggccgcgctctgcgccgggatcctggcagaggcgccccgagtgcgagcccagcacagggagagagtgacctgcacgcgcctttacgccgctgacattgtgttcttactggatggctcctcatccattggccgcagcaatttccgcgaggtccgcagctttctcgaagggctggtgctgcctttctctggagcagccagtgcacagggtgtgcgctttgccacagtgcagtacagcgatgacccacggacagagttcggcctggatgcacttggctctgggggtgatgtgatccgcgccatccgtgagcttagctacaaggggggcaacactcgcacaggggctgcaattctccatgtggctgaccatgtcttcctgccccagctggcccgacctggtgtccccaaggtctgcatcctgatcacagacgggaagtcccaggacctggtggacacagctgcccaaaggctgaaggggcagggggtcaagctatttgctgtggggatcaagaatgctgaccctgaggagctgaagcgagttgcctcacagcccaccagtgacttcttcttcttcgtcaatgacttcagcatcttgaggacactactgcccctcgtttcccggagagtgtgcacgactgctggtggcgtgcctgtgacccgacctccggatgactcgacctctgctccacgagacctggtgctgtctgagccaagcagccaatccttgagagtacagtggacagcggccagtggccctgtgactggctacaaggtccagtacactcctctgacggggctgggacagccactgccgagtgagcggcaggaggtgaacgtcccagctggtgagaccagtgtgcggctgcggggtctccggccactgaccgagtaccaagtgactgtgattgccctctacgccaacagcatcggggaggctgtgagcgggacagctcggaccactgccctagaagggccggaactgaccatccagaataccacagcccacagcctcctggtggcctggcggagtgtgccaggtgccactggctaccgtgtgacatggcgggtcctcagtggtgggcccacacagcagcaggagctgggccctgggcagggttcagtgttgctgcgtgacttggagcctggcacggactatgaggtgaccgtgagcaccctatttggccgcagtgtggggcccgccacttccctgatggctcgcactgacgcttctgttgagcagaccctgcgcccggtcatcctgggccccacatccatcctcctttcctggaacttggtgcctgaggcccgtggctaccggttggaatggcggcgtgagactggcttggagccaccgcagaaggtggtactgccctctgatgtgacccgctaccagttggatgggctgcagccgggcactgagtaccgcctcacactctacactctgctggagggccacgaggtggccacccctgcaaccgtggttcccactggaccagagctgcctgtgagccctgtaacagacctgcaagccaccgagctgcccgggcagcgggtgcgagtgtcctggagcccagtccctggtgccacccagtaccgcatcattgtgcgcagcacccagggggttgagcggaccctggtgcttcctgggagtcagacagcattcgacttggatgacgttcaggctgggcttagctacactgtgcgggtgtctgctcgagtgggtccccgtgagggcagtgccagtgtcctcactgtccgccgggagccggaaactccacttgctgttccagggctgcgggttgtggtgtcagatgcaacgcgagtgagggtggcctggggacccgtccctggagccagtggatttcggattagctggagcacaggcagtggtccggagtccagccagacactgcccccagactctactgccacagacatcacagggctgcagcctggaaccacctaccaggtggctgtgtcggtactgcgaggcagagaggagggccctgctgcagtcatcgtggctcgaacggacccactgggcccagtgaggacggtccatgtgactcaggccagcagctcatctgtcaccattacctggaccagggttcctggcgccacaggatacagggtttcctggcactcagcccacggcccagagaaatcccagttggtttctggggaggccacggtggctgagctggatggactggagccagatactgagtatacggtgcatgtgagggcccatgtggctggcgtggatgggccccctgcctctgtggttgtgaggactgcccctgagcctgtgggtcgtgtgtcgaggctgcagatcctcaatgcttccagcgacgttctacggatcacctgggtaggggtcactggagccacagcttacagactggcctggggccggagtgaaggcggccccatgaggcaccagatactcccaggaaacacagactctgcagagatccggggtctcgaaggtggagtcagctactcagtgcgagtgactgcacttgtcggggaccgcgagggcacacctgtctccattgttgtcactacgccgcctgaggctccgccagccctggggacgcttcacgtggtgcagcgcggggagcactcgctgaggctgcgctgggagccggtgcccagagcgcagggcttccttctgcactggcaacctgagggtggccaggaacagtcccgggtcctggggcccgagctcagcagctatcacctggacgggctggagccagcgacacagtaccgcgtgaggctgagtgtcctagggccagctggagaagggccctctgcagaggtgactgcgcgcactgagtcacctcgtgttccaagcattgaactacgtgtggtggacacctcgatcgactcggtgactttggcctggactccagtgtccagggcatccagctacatcctatcctggcggccactcagaggccctggccaggaagtgcctgggtccccgcagacacttccagggatctcaagctcccagcgggtgacagggctagagcctggcgtctcttacatcttctccctgacgcctgtcctggatggtgtgcggggtcctgaggcatctgtcacacagacgccagtgtgcccccgtggcctggcggatgtggtgttcctaccacatgccactcaagacaatgctcaccgtgcggaggctacgaggagggtcctggagcgtctggtgttggcacttgggcctcttgggccacaggcagttcaggttggcctgctgtcttacagtcatcggccctccccactgttcccactgaatggctcccatgaccttggcattatcttgcaaaggatccgtgacatgccctacatggacccaagtgggaacaacctgggcacagccgtggtcacagctcacagatacatgttggcaccagatgctcctgggcgccgccagcacgtaccaggggtgatggttctgctagtggatgaacccttgagaggtgacatattcagccccatccgtgaggcccaggcttctgggcttaatgtggtgatgttgggaatggctggagcggacccagagcagctgcgtcgcttggcgccgggtatggactctgtccagaccttcttcgccgtggatgatgggccaagcctggaccaggcagtcagtggtctggccacagccctgtgtcaggcatccttcactactcagccccggccagagccctgcccagtgtattgtccaaagggccagaagggggaacctggagagatgggcctgagaggacaagttgggcctcctggcgaccctggcctcccgggcaggaccggtgctcccggcccccaggggccccctggaagtgccactgccaagggcgagaggggcttccctggagcagatgggcgtccaggcagccctggccgcgccgggaatcctgggacccctggagcccctggcctaaagggctctccagggttgcctggccctcgtggggacccgggagagcgaggacctcgaggcccaaagggggagccgggggctcccggacaagtcatcggaggtgaaggacctgggcttcctgggcggaaaggggaccctggaccatcgggcccccctggacctcgtggaccactgggggacccaggaccccgtggccccccagggcttcctggaacagccatgaagggtgacaaaggcgatcgtggggagcggggtccccctggaccaggtgaaggtggcattgctcctggggagcctgggctgccgggtcttcccggaagccctggaccccaaggccccgttggcccccctggaaagaaaggagaaaaaggtgactctgaggatggagctccaggcctcccaggacaacctgggtctccgggtgagcagggcccacggggacctcctggagctattggccccaaaggtgaccggggctttccagggcccctgggtgaggctggagagaagggcgaacgtggacccccaggcccagcgggatcccgggggctgccaggggttgctggacgtcctggagccaagggtcctgaagggccaccaggacccactggccgccaaggagagaagggggagcctggtcgccctggggaccctgcagtggtgggacctgctgttgctggacccaaaggagaaaagggagatgtggggcccgctgggcccagaggagctaccggagtccaaggggaacggggcccacccggcttggttcttcctggagaccctggccccaagggagaccctggagaccggggtcccattggccttactggcagagcaggacccccaggtgactcagggcctcctggagagaagggagaccctgggcggcctggccccccaggacctgttggcccccgaggacgagatggtgaagttggagagaaaggtgacgagggtcctccgggtgacccgggtttgcctggaaaagcaggcgagcgtggccttcggggggcacctggagttcgggggcctgtgggtgaaaagggagaccagggagatcctggagaggatggacgaaatggcagccctggatcatctggacccaagggtgaccgtggggagccgggtcccccaggacccccgggacggctggtagacacaggacctggagccagagagaagggagagcctggggaccgcggacaagagggtcctcgagggcccaagggtgatcctggcctccctggagcccctggggaaaggggcattgaagggtttcggggacccccaggcccacagggggacccaggtgtccgaggcccagcaggagaaaagggtgaccggggtccccctgggctggatggccggagcggactggatgggaaaccaggagccgctgggccctctgggccgaatggtgctgcaggcaaagctggggacccagggagagacgggcttccaggcctccgtggagaacagggcctccctggcccctctggtccccctggattaccgggaaagccaggcgaggatggcaaacctggcctgaatggaaaaaacggagaacctggggaccctggagaagacgggaggaagggagagaaaggagattcaggcgcctctgggagagaaggtcgtgatggccccaagggtgagcgtggagctcctggtatccttggaccccaggggcctccaggcctcccagggccagtgggccctcctggccagggttttcctggtgtcccaggaggcacgggccccaagggtgaccgtggggagactggatccaaaggggagcagggcctccctggagagcgtggcctgcgaggagagcctggaagtgtgccgaatgtggatcggttgctggaaactgctggcatcaaggcatctgccctgcgggagatcgtggagacctgggatgagagctctggtagcttcctgcctgtgcccgaacggcgtcgaggccccaagggggactcaggcgaacagggccccccaggcaaggagggccccatcggctttcctggagaacgcgggctgaagggcgaccgtggagaccctggccctcaggggccacctggtctggcccttggggagaggggcccccccgggccttccggccttgccggggagcctggaaagcctggtattcccgggctcccaggcagggctgggggtgtgggagaggcaggaaggccaggagagaggggagaacggggagagaaaggagaacgtggagaacagggcagagatggccctcctggactccctggaacccctgggccccccggaccccctggccccaaggtgtctgtggatgagccaggtcctggactctctggagaacagggaccccctggactcaagggtgctaagggggagccgggcagcaatggtgaccaaggtcccaaaggagacaggggtgtgccaggcatcaaaggagaccggggagagcctggaccgaggggtcaggacggcaacccgggtctaccaggagagcgtggtatggctgggcctgaagggaagccgggtctgcagggtccaagaggcccccctggcccagtgggtggtcatggagaccctggaccacctggtgccccgggtcttgctggccctgcaggaccccaaggaccttctggcctgaagggggagcctggagagacaggacctccaggacggggcctgactggacctactggagctgtgggacttcctggaccccccggcccttcaggccttgtgggtccacaggggtctccaggtttgcctggacaagtgggggagacagggaagccgggagccccaggtcgagatggtgccagtggaaaagatggagacagagggagccctggtgtgccagggtcaccaggtctgcctggccctgtcggacctaaaggagaacctggccccacgggggcccctggacaggctgtggtcgggctccctggagcaaagggagagaagggagcccctggaggccttgctggagacctggtgggtgagccgggagccaaaggtgaccgaggactgccagggccgcgaggcgagaagggtgaagctggccgtgcaggggagcccggagaccctggggaagatggtcagaaaggggctccaggacccaaaggtttcaagggtgacccaggagtcggggtcccgggctcccctgggcctcctggccctccaggtgtgaagggagatctgggcctccctggcctgcccggtgctcctggtgttgttgggttcccgggtcagacaggccctcgaggagagatgggtcagccaggccctagtggagagcggggtctggcaggccccccagggagagaaggaatcccaggacccctggggccacctggaccaccggggtcagtgggaccacctggggcctctggactcaaaggagacaagggagaccctggagtagggctgcctgggccccgaggcgagcgtggggagccaggcatccggggtgaagatggccgccccggccaggagggaccccgaggactcacggggccccctggcagcaggggagagcgtggggagaagggtgatgttgggagtgcaggactaaagggtgacaagggagactcagctgtgatcctggggcctccaggcccacggggtgccaagggggacatgggtgaacgagggcctcggggcttggatggtgacaaaggacctcggggagacaatggggaccctggtgacaagggcagcaagggagagcctggtgacaagggctcagccgggttgccaggactgcgtggactcctgggaccccagggtcaacctggtgcagcagggatccctggtgacccgggatccccaggaaaggatggagtgcctggtatccgaggagaaaaaggagatgttggcttcatgggtccccggggcctcaagggtgaacggggagtgaagggagcctgtggccttgatggagagaagggagacaagggagaagctggtcccccaggccgccccgggctggcaggacacaaaggagagatgggggagcctggtgtgccgggccagtcgggggcccctggcaaggagggcctgatcggtcccaagggtgaccgaggctttgacgggcagccaggccccaagggtgaccagggcgagaaaggggagcggggaaccccaggaattgggggcttcccaggccccagtggaaatgatggctctgctggtcccccagggccacctggcagtgttggtcccagaggccccgaaggacttcagggccagaagggtgagcgaggtccccccggagagagagtggtgggggctcctggggtccctggagctcctggcgagagaggggagcaggggcggccagggcctgccggtcctcgaggcgagaagggagaagctgcactgacggaggatgacatccggggctttgtgcgccaagagatgagtcagcactgtgcctgccagggccagttcatcgcatctggatcacgacccctccctagttatgctgcagacactgccggctcccagctccatgctgtgcctgtgctccgcgtctctcatgcagaggaggaagagcgggtaccccctgaggatgatgagtactctgaatactccgagtattctgtggaggagtaccaggaccctgaagctccttgggatagtgatgacccctgttccctgccactggatgagggctcctgcactgcctacaccctgcgctggtaccatcgggctgtgacaggcagcacagaggcctgtcacccttttgtctatggtggctgtggagggaatgccaaccgttttgggacccgtgaggcctgcgagcgccgctgcccaccccgggtggtccagagccaggggacaggtactgcccaggactgaggcccagataatgagctgagattcagcatcccctggaggagtcggggtctcagcagaaccccactgtccctccccttggtgctagaggcttgtgtgcacgtgagcgtgcgtgtgcacgtccgttatttcagtgacttggtcccgtgggtctagccttcccccctgtggacaaacccccattgtggctcctgccaccctggcagatgactcactgtgggggggtggctgtgggcagtgagcggatgtgactggcgtctgacccgccccttgacccaagcctgtgatgacatggtgctgattctggggggcattaaagctgctgttttaaaaggc

(19) United States
(12) Patent Application Publication
(10) Pub. No.: US 2024/0424065 A1
(43) Pub. Date: Dec. 26, 2024

(54) COLLAGEN 7 PROTEIN REPLACEMENT THERAPY

(71) Applicant: Phoenix Tissue Repair, Inc., Boston, MA (US)

(72) Inventors: Hal LANDY, Jupiter, FL (US); Sanuj RAVINDRAN, San Francisco, CA (US)

(21) Appl. No.: 18/700,333

(22) PCT Filed: Oct. 12, 2022
(86) PCT No.: PCT/US2022/077964

§ 371 Date: Apr. 11, 2024

Related U.S. Application Data
(60) Provisional application No. 63/254,953, filed on Oct. 12, 2021.
(60) Provisional application No. 63/342,993, filed on May 17, 2022.

(51) Int. Cl. A61K 38/39; A61P 17/02

(52) U.S. Cl. CPC A61K 38/39; A61P 17/02

(57) ABSTRACT

The present disclosure relates to collagen 7 (Col7) protein replacement therapy for treatment of a skin disorder, specifically dystrophic epidermolysis bullosa (DEB). Recombinant collagen 7 drug products. compositions, and methods for administration and monitoring the outcomes of the treatment are provided.

### FIELD OF THE DISCLOSURE

The present disclosure relates to recombinant collagen 7 protein therapy for Dystrophic Epidermolysis Bullosa (DEB), in particular Recessive Dystrophic Epidermolysis Bullosa (RDEB).

### REFERENCE TO THE SEQUENCE LISTING

The present application is being filed along with a Sequence Listing in electronic format. The Sequence Listing is provided as a file entitled 51624-014WO3_Sequence_Listing_10_7_22.xml, created on Oct. 7, 2022, which is 14,778 bytes in size. The information in the electronic format of the sequence listing is incorporated herein by reference in its entirety.

### BACKGROUND OF THE DISCLOSURE

Epidermolysis bullosa (EB) encompasses a group of molecularly diverse diseases characterized by the development of blisters after minor mechanical trauma to the skin. Almost all patients experience severe, recurrent blistering or ruptured skin, although severity, extracutaneous manifestations, and clinical course depend on the type of EB. The four major types of inherited EB include EB simplex, junctional EB, dystrophic EB (DEB), and Kindler syndrome and are differentiated based on the level of the skin and/or noncutaneous tissue where the absent or affected protein is located and where the blisters and their clinical manifestations develop.

DEB, one of the most severe forms of EB, is characterized by recurrent blistering with subsequent healing and scarring. In patients with DEB, blistering can be triggered by even minor mechanical trauma owing to the extremely fragile nature of the skin. This leads to a chronic cycle of blistering, healing, and re-blistering that causes patients to suffer from painful wounds and debilitating scarring of epithelial tissue.

DEB is caused by mutations in the COL7A1 gene, which encodes the alpha chain of type VII collagen (Col7), a protein essential for the formation of the fibrils that anchor the basement membrane to the underlying dermis. The condition is inherited as either a dominant (DDEB) or a recessive form (RDEB). The recessive form typically presents with a more severe phenotype and in the most severe generalized form of RDEB, wounds affecting the whole body may be present. The correlation between genotype and phenotype of RDEB and COL7A1 mutations is highly variable.

The extent of cutaneous involvement ranges from localized blistering of the extremities to widespread lesions. It is common for patients with all forms of DEB to have dystrophy or loss of nails. Scarring of the hands and feet causing webbing between the digits known as pseudosyndactyly, with progression to mutilating deformities in which the fingers or toes are encased in scar tissue, is most characteristic of severe, generalized RDEB, while pseudosyndactyly is infrequent in DDEB.

The behavior of wounds in DEB, particularly in more severe forms of the disease RDEB, has several distinct characteristics. The wounds are dynamic and occur due to the skin's impaired mechanical resistance to external shear forces; they heal but may recur, reflecting continued risk for mechanical injury that led to the first wound as well as alterations in the healing process due to Col7 deficiency. Characteristic features of DEB include life-long skin fragility and healing with scarring, apparently due to effects on two interconnected Col7-dependent mechanisms.

In addition to the cutaneous symptoms, severe forms of DEB can cause erosion and scarring of mucous membranes such as those of the eye, mouth and esophagus, genitals, and anus; dental abnormalities are also common. Oral involvement may lead to mouth blistering. fusion of the tongue to the floor of the mouth, and progressive diminution of the size of the oral cavity. Esophageal erosions can lead to webs and strictures that can cause severe dysphagia. Consequently, nutritional deficiency, anemia, and other secondary problems are common. Corneal erosions can lead to scarring and loss of vision. Blistering of the hands and fect followed by scarring results in pseudosyndactyly, a hallmark of this disorder. The lifetime risk of aggressive squamous cell carcinoma is higher than 90%. Other forms of DEB have similar, but less pronounced clinical findings.

Pathogenic mutations in the COL7A1 gene lead to abnormal, decreased or a complete absence of anchoring fibrils in the skin, resulting in DEB. This lack of normal functioning anchoring fibrils is responsible for poor epidermal-dermal adherence, pronounced skin fragility and severe blistering in the skin and mucosa in DEB patients.

There is no definitive treatment for DEB and its management focuses on supportive care. Bandaging and infection prevention are the main strategies for management of all forms of EB, along with pain and itch relief, nutritional support, and surgical management as required. Current standard of care with allografts is of very limited benefit in RDEB patients, as 0% of wounds in RDEB were reported healed after 16 weeks of grafting because of the chronic and dynamic nature of wounds in RDEB patients. There is, however, no uniformly accepted standard of care: the choice of bandages, routine, and distraction techniques differs among families and centers. Severely affected patients with wide-ranging cutaneous and extracutaneous symptoms also require a multidisciplinary approach to treatment. Patients with DEB experience diminished health-related quality of life (HRQL) due to pain, pruritus, decreased activities of daily living, fatigue, reduced weight, and the social impact of disease manifestations.

DEB is a devastating systemic disease with no effective therapy. No systemic disease modifying approaches are known to be in development. Because DEB's debilitating effects arise from deficiencies in functional Col7, DEB is appropriate for systemic protein therapy with recombinant human Col7 (rhCol7). The present disclosure provides rCol7 (e.g., rhCol7) protein therapies for systemically treating DEB.

### SUMMARY OF THE DISCLOSURE

The present disclosure relates to collagen 7 (alternately referred to herein as col7, Col7, or C7), specifically recombinant human collagen 7 (alternately referred to herein as rhCol7 or rhC7) protein replacement therapy for treatment of dystrophic epidermolysis bullosa (DEB), especially recessive DEB (RDEB). The treatment relates to systemic administration of a recombinant collagen 7 (e.g., rCol7, such as rhCol7) substance to subjects with DEB by intravenous administration, e.g., intravenous infusion. The present disclosure is based in part on the premise that rCol7 protein is able to deposit within wounded and unwounded skin sites, specifically concentrating at the dermal-epidermal junction (DEJ), through systemic administration, and thus can be used to treat, prevent, delay the onset of, ameliorate a complication and/or symptom of, and/or prevent the progression of a clinical complication and/or symptom associated with DEB.

In an aspect, the present disclosure provides a method of treating dystrophic epidermolysis bullosa (DEB) in a subject in need thereof, comprising intravenously administering to the subject a recombinant human Col7 (rhCol7) substance according to a first dosing regimen and a second dosing regimen, wherein: i) the first dosing regimen includes administering a first therapeutically effective amount of the substance at a first frequency; and ii) the second dosing regimen includes administering a second therapeutically effective amount of the substance at a second frequency, wherein the first frequency is greater than the second frequency, and wherein administration according to the first dosing regimen occurs prior to administration according to the second dosing regimen.

In another aspect, the present disclosure provides a method of ameliorating. alleviating, reducing, improving, delaying onset of, delaying progression of, eliminating, and/or curing a symptom or complication of dystrophic epidermolysis bullosa (DEB) in a subject in need thereof, comprising intravenously administering to the subject a recombinant human Col7 (rhCol7) substance according to a first dosing regimen and a second dosing regimen, wherein: i) the first dosing regimen includes administering a first therapeutically effective amount of the substance at a first frequency; and ii) the second dosing regimen includes administering a second therapeutically effective amount of the substance at a second frequency, wherein the first frequency is greater than the second frequency, and wherein administration according to the first dosing regimen occurs prior to administration according to the second dosing regimen.

In a further aspect, the present disclosure provides a Col7 substance (e.g., a recombinant human Col7, rhCol7) for use in a method of treating dystrophic epidermolysis bullosa (DEB) in a subject in need thereof, the method comprising administering to the subject a recombinant human Col7 (rhCol7) substance according to a first dosing regimen and a second dosing regimen, wherein: i) the first dosing regimen includes administering a first therapeutically effective amount of the substance at a first frequency; and ii) the second dosing regimen includes administering a second therapeutically effective amount of the substance at a second frequency, wherein the first frequency is greater than the second frequency, and wherein administration according to the first dosing regimen occurs prior to administration according to the second dosing regimen.

In a related aspect, the present disclosure provides a Col7 substance (e.g., a recombinant human Col7, rhCol7) for use in a method of ameliorating, alleviating, reducing, improving, delaying onset of, delaying progression of, eliminating, and/or curing a symptom or complication of dystrophic epidermolysis bullosa (DEB) in a subject in need thereof, the method comprising intravenously administering to the subject a recombinant human Col7 (rhCol7) substance according to a first dosing regimen and a second dosing regimen, wherein: i) the first dosing regimen includes administering a first therapeutically effective amount of the substance at a first frequency; and ii) the second dosing regimen includes administering a second therapeutically effective amount of the substance at a second frequency, wherein the first frequency is greater than the second frequency, and wherein administration according to the first dosing regimen occurs prior to administration according to the second dosing regimen.

In a related aspect, the present disclosure provides a use of a Col7 substance (e.g., a recombinant human Col7, rhCol7) for the manufacture of a medicament for the treatment of dystrophic epidermolysis bullosa (DEB) or a symptom or complication thereof, wherein the treatment comprises administering to a subject in need thereof a recombinant human Col7 (rhCol7) substance according to a first dosing regimen and a second dosing regimen, wherein: i) the first dosing regimen includes administering a first therapeutically effective amount of the substance at a first frequency; and ii) the second dosing regimen includes administering a second therapeutically effective amount of the substance at a second frequency, wherein the first frequency is greater than the second frequency, and wherein administration according to the first dosing regimen occurs prior to administration according to the second dosing regimen.

In a related aspect, the present disclosure provides a use of a Col7 substance (e.g., a recombinant human Col7, rhCol7) for the manufacture of a medicament for the amelioration, alleviation, reduction, improvement, delay of onset of, delay of progression of, elimination, and/or curing of a symptom or complication of dystrophic epidermolysis bullosa (DEB), wherein the amelioration, alleviation, reduction, improvement, delay of onset of, delay of progression of, elimination, and/or curing comprises administering to a subject in need thereof a recombinant human Col7 (rhCol7) substance according to a first dosing regimen and a second dosing regimen, wherein: i) the first dosing regimen includes administering a first therapeutically effective amount of the substance at a first frequency; and ii) the second dosing regimen includes administering a second therapeutically effective amount of the substance at a second frequency, wherein the first frequency is greater than the second frequency, and wherein administration according to the first dosing regimen occurs prior to administration according to the second dosing regimen.

In some embodiments of the preceding aspects, the symptom or complication of DEB is skin wounds including blistering, itching, pain, skin erosion, scarring, skin fragility, gangrene, aplasia or hypoplasia of the skin, hypopigmentation of skin, milia, skin infections, cheilitis (e.g., inflammation of lips), dystrophic fingernails or toenails, abnormal dental enamel, pseudosyndactyly or camptodactyly of fingers or toes, finger or toe syndactyly, flexion contracture of toes, carious teeth, dysphagia (e.g., poor swallowing), furrowed or grooved tongue, esophageal stricture, laryngeal stenosis, failure to thrive, dilated cardiomyopathy, abnormal pulmonary interstitial morphology, acute constipation, hearing or visual impairment, eczema, glomerulopathy, immunologic hypersensitivity, nasolacrimal duct obstruction, anemia, chronic ear infections, corneal erosion, corneal abrasions and scarring, microstomia, osteopenia, ectropion, immunologic hypersensitivity, nephrotic syndrome, phimosis, renal insufficiency, urinary retention, ureteral stenosis, stroke, squamous cell carcinoma, or a combination thereof. In some embodiments, the symptom or complication of DEB is skin wounds including blistering, skin erosion, scarring, skin fragility, aplasia or hypoplasia of the skin, cheilitis (e.g., inflammation of lips), dystrophic fingernails or toenails, pseudosyndactyly or camptodactyly of fingers or toes, finger or toe syndactyly, flexion contracture of toes, dysphagia (e.g., poor swallowing), esophageal stricture, laryngeal stenosis, or a combination thereof. In some embodiments, the symptom or complication of DEB is skin wounds including blistering.

In some embodiments of the preceding aspects, the method ameliorates, alleviates, reduces, improves, or eliminates one or more skin wounds.

In some embodiments of the preceding aspects, the first dosing regimen lasts at least one week. In some embodiments, the first dosing regimen lasts at least two weeks. In some embodiments, the first dosing regimen lasts at least three weeks. In some embodiments, the first dosing regimen lasts at least four weeks. In some embodiments, the first frequency is about daily, about every two days, about every three days, about every four days, about every five days, about every six days, about every week, about every ten days, or about every two weeks. In some embodiments, the first frequency is weekly.

In some embodiments of the preceding aspects, the second dosing regimen lasts at least four weeks. In some embodiments, the second dosing regimen lasts at least six weeks. In some embodiments, the second dosing regimen lasts at least seven weeks. In some embodiments, the second dosing regimen lasts at least eight weeks. In some embodiments, the second dosing regimen lasts at least six months. In some embodiments, the second dosing regimen lasts at least one year. In some embodiments, the second dosing regimen comprises administering the rhCol7 substance to the subject over the subject's lifetime. In some embodiments, the second frequency is about every week, about every ten days, about every two weeks, about every three weeks, about every four weeks, or about every month. In some embodiments, the second frequency is weekly. In some embodiments, the second frequency is every other week. In some embodiments, the second dosing regimen is chronic (e.g., lasts the subject's lifetime).

In some embodiments of the preceding aspects, the first dosing regimen lasts between one week and four weeks, and the second dosing regimen lasts at least six weeks. In some embodiments, the first dosing regimen lasts one week. In some embodiments, the first dosing regimen lasts two weeks. In some embodiments, the first dosing regimen lasts three weeks. In some embodiments, the first dosing regimen lasts four weeks. In some embodiments, the second dosing regimen lasts seven weeks. In some embodiments, the second dosing regimen lasts at least six months. In some embodiments, the second dosing regimen comprises administering the rhCol7 substance to the subject over the subject's lifetime. In some embodiments, the first dosing regimen lasts four weeks, and the second dosing regimen lasts at least seven weeks.

In some embodiments of the preceding aspects, the first effective amount is about 0.01 mg/kg, 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg 1.2 mg/kg, 1.3 mg/kg, 1.4 mg/kg, 1.5 mg/kg, 1.6 mg/kg, 1.7 mg/kg, 1.8 mg/kg, 1.9 mg/kg. 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg. 3.3 mg/kg. 3.4 mg/kg, 3.5 mg/kg, 3.6 mg/kg, 3.7 mg/kg, 3.8 mg/kg, 3.9 mg/kg, 4.0 mg/kg, 4.5 mg/kg, 5.0 mg/kg, 6.0 mg/kg, 7.0 mg/kg, 8.0 mg/kg, 9.0 mg/kg, 10 mg/kg, 15 mg/kg, or 20 mg/kg. In some embodiments, the first effective amount is about 0.1mg/kg, 0.3 mg/kg, 0.5 mg/kg, 1.0 mg/kg, 1.5 mg/kg, 2.0 mg/kg, or 3.0 mg/kg. In some embodiments, the second effective amount is about 0.1 mg/kg. In some embodiments, the first effective amount is about 0.3 mg/kg. In some embodiments, the second effective amount is about 0.5 mg/kg. In some embodiments, the second effective amount is about 1.0 mg/kg. In some embodiments, the second effective amount is about 1.5 mg/kg. In some embodiments, the second effective amount is about 2.0 mg/kg. In some embodiments, the first effective amount is about 3.0 mg/kg.

In some embodiments of the preceding aspects, the second effective amount is about 0.01 mg/kg. 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg 1.2 mg/kg, 1.3 mg/kg, 1.4 mg/kg, 1.5 mg/kg, 1.6 mg/kg. 1.7 mg/kg, 1.8 mg/kg, 1.9 mg/kg, 2.0 mg/kg, 2.1 mg/kg, 2.2 mg/kg. 2.3 mg/kg, 2.4 mg/kg, 2.5 mg/kg, 2.6 mg/kg, 2.7 mg/kg. 2.8 mg/kg, 2.9 mg/kg. 3.0 mg/kg, 3.1 mg/kg, 3.2 mg/kg. 3.3 mg/kg, 3.4 mg/kg, 3.5 mg/kg. 3.6 mg/kg, 3.7 mg/kg. 3.8 mg/kg, 3.9 mg/kg, 4.0 mg/kg, 4.5 mg/kg, 5.0 mg/kg, 6.0 mg/kg, 7.0 mg/kg, 8.0 mg/kg, 9.0 mg/kg, 10 mg/kg. 15 mg/kg, or 20 mg/kg. In some embodiments, the second effective amount is about 0.1 mg/kg, 0.3 mg/kg, 0.5 mg/kg, 1.0 mg/kg, 1.5 mg/kg, 2.0 mg/kg, or 3.0 mg/kg. In some embodiments, the second effective amount is about 0.1 mg/kg. In some embodiments, the second effective amount is about 0.3 mg/kg. In some embodiments, the second effective amount is about 0.5 mg/kg. In some embodiments, the second effective amount is about 1.0 mg/kg. In some embodiments, the second effective amount is about 1.5 mg/kg. In some embodiments, the second effective amount is about 2.0 mg/kg. In some embodiments, the second effective amount is about 3.0 mg/kg.

In some embodiments of the preceding aspects, the first effective amount is the same as the second effective amount. In some embodiments, the first effective amount and the second effective amount are independently about 0.01 mg/kg. 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg. 1.0 mg/kg. 1.1 mg/kg 1.2 mg/kg, 1.3 mg/kg, 1.4 mg/kg, 1.5 mg/kg, 1.6 mg/kg, 1.7 mg/kg, 1.8 mg/kg. 1.9 mg/kg, 2.0 mg/kg, 2.1 mg/kg. 2.2 mg/kg, 2.3 mg/kg, 2.4 mg/kg. 2.5 mg/kg. 2.6 mg/kg, 2.7 mg/kg, 2.8 mg/kg, 2.9 mg/kg, 3.0 mg/kg, 3.1 mg/kg, 3.2 mg/kg, 3.3 mg/kg. 3.4 mg/kg, 3.5 mg/kg. 3.6 mg/kg. 3.7 mg/kg. 3.8 mg/kg, 3.9 mg/kg, 4.0 mg/kg, 4.5 mg/kg, 5.0 mg/kg, 6.0 mg/kg, 7.0 mg/kg, 8.0 mg/kg, 9.0 mg/kg, 10 mg/kg, 15 mg/kg, or 20 mg/kg. In some embodiments, the first effective amount and the second effective amount are independently about 0.1 mg/kg. 0.3 mg/kg, 0.5 mg/kg, 1.0 mg/kg, 1.5 mg/kg, 2.0 mg/kg, or 3.0 mg/kg. In some embodiments, the first effective amount and the second effective amount are each about 0.3 mg/kg. In some embodiments, the first effective amount and the second effective amount are each about 3.0 mg/kg.

In some embodiments of the preceding aspects, the first dosing regimen lasts at least one week, the first frequency is weekly, and the first effective amount is about 0.1 mg/kg. 0.3 mg/kg, 0.5 mg/kg, 1.0 mg/kg, 1.5 mg/kg, 2.0 mg/kg, or 3.0 mg/kg. In some embodiments, the first dosing regimen lasts four weeks. In some embodiments, the first effective amount is about 0.3 mg/kg. In some embodiments, the first effective amount is about 3.0 mg/kg. In some embodiments, the second dosing regimen lasts at least four weeks, the second frequency is every other week, and the second effective amount is about 0.1 mg/kg. 0.3 mg/kg, 0.5 mg/kg, 1.0 mg/kg, 1.5 mg/kg, 2.0 mg/kg, or 3.0 mg/kg. In some embodiments, the second dosing regimen lasts at least seven weeks. In some embodiments, the second dosing regimen is chronic. In some embodiments, the second dosing regimen lasts over the patient's lifetime. In some embodiments, the second effective amount is about 0.3 mg/kg. In some embodiments, the second effective amount is about 3.0 mg/kg.

In some embodiments of the preceding aspects, the intravenous administration is by intravenous infusion, or slow push injection.

In some embodiments of the preceding aspects, the subject has recessive dystrophic epidermolysis bullosa (RDEB).

In some embodiments of the preceding aspects, the subject has dominant dystrophic epidermolysis bullosa (DDEB).

In some embodiments of the preceding aspects, the subject has a mutation in the COL7A1 gene that is consistent with a recessive inheritance pattern.

In some embodiments of the preceding aspects, the subject is a human. In some embodiments, the subject is under 18 years of age. In some embodiments, the subject is at least 6 years of age. In some embodiments, the subject is at least 2 years of age. In some embodiments, the subject is less than 2 years of age. In some embodiments, the subject is at least 18 years of age.

In some embodiments of the preceding aspects, the rhCol7 substance is included in a pharmaceutical composition comprising 1.2 mg/mL rhCol7 substance, 10 mM sodium phosphate, 5 mM sodium citrate, 100 mM L-arginine, 1.7% sucrose (w/v), 70 mM sodium chloride, and 0.05% (v/v) polysorbate 20 (pH 7.2).

In some embodiments of the preceding aspects, the second dosing regimen duration, second frequency, and/or second effective amount is determined at least in part based on an assessment of one or more clinical parameters of the subject performed during or after the first dosing regimen. In some embodiments, the method further comprises, between the first dosing regimen and the second dosing regimen, assessing one or more clinical parameters. In some embodiments, the method further comprises, during the first dosing regimen, assessing one or more clinical parameters. In some embodiments, the method further comprises, during the second dosing regimen, assessing one or more clinical parameters. In some embodiments, the one or more clinical parameters are selected from wound surface area, wound healing, time to chronic wound healing, and time in re-blistering.

### BRIEF SUMMARY OF THE DRAWINGS

FIG. 1 shows representative images of dose dependent deposition of Col7 in tongue and skin in Col7a1−/− mice following three intravenous injections.

FIG. 2A shows representative images (Hematoxylin & Eosin staining) of tongue tissues in Col7a1−/− mice following a single intravenous administration of rhCol7.

FIG. 2B is a histogram of the portion of Col7a1−/− mice with closure of dermal-epidermal separation in tongue following as single administration of Col7.

FIG. 3 shows the PTR-01 exposure-dependent deposition of Col7. All the patients received rCol7 replacement achieved increases in Col7 at or above 35% normal human skin (NHS). Ph2 patients (black data points) received 4×3 mg/kg weekly (1-29 days), plus 7×3 mg/kg every other week (42-120 days) vs. Ph1 patients received only 3×PTR-01 doses bi-weekly (1-29 days). *Day 22 and 120 unwounded samples for 202-001 were poor quality, no data—data shown is for wounded sample. Patients 02-06, 02-07, 02-08 participated in the Phase 1/2 study. NHS: normal human skin.

FIG. 4 schematically illustrates the study design for a Phase 2, Open Label study of PTR-01. qow: every other week.

FIG. 5 shows the comparison of Day 120 wound evaluations vs. baseline.

FIG. 6 shows the wound response by percent reduction in wound surface area by Canfield imaging. *N wound images were not available for Day 36 (wound 6; patient 201-001) and Day 92 (wound 1; patient 201-002); therefore, wound surface areas for these patients at those time points were omitted. Numbers in the shaded bars represent the number of wounds in each percentage category; the number above each bar is the total number of wounds assessed at that timepoint.

FIG. 7 shows the wound closure observed in chronic and recurrent wounds.

FIG. 8 shows example wound images demonstrating clear reduction in wound surface area post PTR-01 treatment.

FIGS. 9A-9B show individual wound and median change from baseline in wound surface area. AUC: area under the curve (cumulative wound surface area from baseline); N/A: not applicable; *Hodges-Lehmann estimate of difference in medians=−18.01, 95% CI=(−40.35,7.19); Wilcoxon rank-sum p=0.1776.

FIGS. 10A-10B show improvements in pain, disease impact, activities of daily living, mood, and essential functions measured by iscorEB-P by patients in the Phase 2 study. iscorEB-P: Patient reported assessment of iscorEB instrument.

FIG. 11 shows Investigator and Patient Global Impression of Change (GIC) score by patient and time point. *Data point missing. Patient 203-001 is not shown. The number above each column is the number of patients with that score and the height of the column reflects the IGIC or PGIC score. Note that the only decline in perceived global status occurred in patient 203-002 at 3 months after the end of treatment. IGI: Investigator Global Impression; PGI: Patient Global Impression.

FIG. 12 shows reduction in skin pro-fibrotic biomarker staining with PTR-01administration. ¹Scoring was performed on a 3-point scale (1=low; 2=moderate; 3=high). N=5 patients (study completers).

### DETAILED DESCRIPTION OF THE DISCLOSURE

The present disclosure provides methods of treating a skin disorder (e.g., dystrophic epidermolysis bullosa (DEB), such as recessive DEB), and/or ameliorating, alleviating, reducing, improving, delaying onset of, delaying progression of, eliminating, and/or curing a symptom or complication of a skin disorder such as DEB in a subject (e.g., a human subject) in need thereof. The methods provided herein may involve administering a pharmaceutical composition comprising a collagen 7 (e.g., C7, col7, or Col7, such as a recombinant collagen 7 (rCol7), such as a recombinant human collagen 7 (rhCol7)), or a functional variant thereof, to the subject according to a first dosing regimen and a second dosing regimen. The first dosing regimen may be a “loading” dosing phase in which a subject is provided a higher dose of collagen 7 and/or is administered collagen 7 at a higher frequency than in the second dosing regimen. The second dosing regimen may be a “maintenance” dosing phase in which the subject is provided a lower dose of collagen 7 and/or is administered the collagen 7 at a lower frequency than in the first dosing regimen. The use of distinct dosing regimens may serve to acclimate the subject to the therapy and address acute symptoms during the loading phase, and to maintain the therapeutic benefits of the collagen 7 therapy and prevent the development or worsening of symptoms during the maintenance phase. The loading phase may last for one or more days, weeks, or months, while the maintenance phase will generally be longer than the loading phase and may last for weeks, months, or years, and may extend through the subject's lifetime.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present disclosure will control.

### INTRODUCTION

DEB patients lack functional collagen 7 due to mutations in the COL7A1 gene which encodes type VII collagen (also referred to as C7 or Col7). Col7 is required to produce anchoring fibrils necessary for epidermis-dermis adhesion. Endogenous Col7 forms through a complex supramolecular aggregation process. Specifically, three alpha chains associate through their C-terminus ends to form a homo-trimer molecule, which folds into a triple helical conformation within the collagenous region. Two homo-trimers form anti-parallel dimers, from which the C-terminal propeptides are proteolytically cleaved by proteases such as bone morphogenetic protein 1. Subsequently, a large number of these anti-parallel dimers laterally aggregate to form anchoring fibrils. Col7 acts as a major component of the anchoring fibrils enabling the attachment of the epidermis to the dermis. A mutation in the COL7Al gene involved in DEB (e.g., RDEB) leads to aberrant synthesis of C7 or defective assembly of the protein into anchoring fibrils, resulting in poor epidermal-dermal adherence.

Replacing the aberrant or missing protein with systemic, intravenous-administered collagen 7, such as a recombinant Col7 (rCol7), is predicted to allow formation of anchoring fibrils and therefore lead to correction of blistering abnormalities and complications throughout the body in patients with DEB (e.g., RDEB). Intravenous administration of rCol7 in mice has been demonstrated to result in deposition of collagen 7 protein in the skin and improvement in wound healing (see, e.g., U.S. Patent Publication No. US20140031295, which is herein incorporated in reference in its entirety).

The present disclosure relates to a disease-modifying Col7 (e.g., rCol7, such as rhCol7) replacement therapy for subjects with DEB, in particular subjects with recessive DEB (RDEB). A rCol7 composition (PTR-01) is developed by Applicant and used for treatment of patients with DEB, especially patients with RDEB. Systemic (e.g., intravenous) delivery of rCol7 is believed to restore functional Col7 to the dermal-epidermal basement membrane zone (BMZ), thereby promoting the assembly of normal anchoring fibrils, providing stability to dermal-epidermal adhesion at the lamina densa/upper papillary dermis interface, and correcting blistering abnormalities and complications in patients with DEB.

### DEFINITIONS

The terms used in this disclosure generally have their ordinary meanings in the art, within the context of this disclosure and in the specific context where each term is used.

As used herein, the terms “Collagen 7,” “Col7,” “Collagen Type VII,” and “C7” are used interchangeably and refer to collagen 7 protein. Human Col7 (also referred to herein as hCol7) is a large extracellular protein with a mass of approximately 900 kilodaltons (kDa) that is a major component of the anchoring fibrils enabling the attachment of the epidermis to the dermis. Col7 is a homo-trimer consisting of three human collagen alpha-1 (Type VII) polypeptides (alpha-1 chains). The alpha-1 chain (Type VII) is encoded by the COL7A1 genc. Each Col7 alpha-1 chain is 2928 amino acids in length and comprises a central collagenous domain flanked by two non-collagenous domains: the NC1 domain at the N-terminus and the NC-2 domain at the C-terminus. Three alpha-1 chains associate through their C-terminal NC2 domains to form the homo-trimer molecule which folds into a triple-helical conformation within the collagenous region, which contains hydroxylated proline and lysine residues. Two homo-trimers lead to the formation of an antiparallel dimer with the N-terminus (NC1 domain) present at both ends of the anti-parallel dimer. Lateral assembly of anti-parallel dimers lead to the formation of the anchoring fibrils that are highly specialized attachment structures in the basement membrane zone (BMZ) and are critical for the attachment of the epidermis to the underlying dermis. These anchoring fibrils could also extend from the lamina densa of the BMZ to the upper papillary dermis within the dermal-epidermal junction in normal skin. The full-length alpha chain polypeptide of human Col7 comprises the amino acid sequence of SEQ ID NO: 1 (Ref. NO.: NP_000085), which is naturally encoded by the nucleic acid sequence of SEQ ID NO: 2 (Ref. NO.: NM_000094). In the context of the present disclosure, Col7 may refer to recombinant collagen 7 protein, or a functional variant thereof. A functional variant of a rCol7 may have structural properties (e.g., anchoring fibril formation), binding properties (e.g., binding to collagen IV and/or fibronectin), and/or signal transduction activities (e.g., tumor growth factor beta (TGF-beta) suppression and/or fibroblast growth factor 2 (FGF2) expression) equivalent to or substantially similar to full length Col7. As used herein, the term “recombinant human Col7 (rhCol7)” refers to a recombinant form of human Col7 having at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to the amino acid sequence of a native human Col7 (e.g., SEQ ID NO: 1). A recombinant Col7 (e.g., rhCol7) may be made by recombinant techniques, such as made by recombinant DNA methods and produced in a host cell comprising a nucleotide sequence encoding Col7. The host cell may be a mammalian cell that provides necessary modifications to recombinant Col7.

As used herein, the terms “recombinant human collagen 7 substance,” “rhCol7 substance,” and “rhC7 substance” are used interchangeably and refer to a substance (e.g., a drug substance) comprising a plurality of recombinant Col7 alpha polypeptides, and/or functional variants thereof. In some embodiments, an rhCol7 substance comprises a recombinant Col7 alpha polypeptide having an amino acid sequence of SEQ ID NO: 1. An rhCol7 substance may be produced by host cells that are engineered to express high levels of recombinant Col7 and/or functional variants thereof. The rhCol7 substance may be purified from the culture media of the host cells. Suitable host cells may include primary or transformed cell lines, including, but not limited to, fibroblasts, keratinocytes, CHO cells, HEK293 cells, C127 cells, VERO cells, BHK cells, HeLa cells, COS cells, MDCK cells, etc.

As used herein, the term “recombinant human Col7 (rhCol7) drug product” refers to a drug formulation comprising rhCol7 substance as the active ingredient. An rhCol7 drug product is formulated to maintain Col7 protein stability and is suitable for clinical administration by, for example, intravenous injection and/or infusion. An rhCol7 drug product may be a pharmaceutical composition comprising an rhCol7 substance.

As used herein, the term “protein therapy” refers to a medical treatment that supplements or replaces a protein in subjects in which that particular protein is deficient or absent. The protein may be introduced into the subject through gene therapy, cell therapy, and/or direct protein replacement. The term “protein replacement” refers to the introduction of a non-native, purified protein into a subject in which such protein is deficient or absent. The administered protein may be obtained from natural sources such as from subjects having normal protein, by, e.g., purification of protein from isolated tissue or fluid such as placenta and animal milk, or by recombinant protein expression. The purified, recombinant protein may be produced in vitro, e.g., using engineered cell lines. Protein replacement therapy may also refer to the introduction of a purified protein to a subject otherwise requiring or benefiting from administration of a purified protein, e.g., suffering from protein insufficiency.

As used herein, the term “mutant protein” refers to a protein translated from a gene containing one or more genetic mutations that result in an altered protein sequence, a truncated protein fragment, and/or complete absence of the protein. In contrast, a “wild type” or “native” protein refers to any protein encoded by a wild-type gene that is capable of having normal functional biological activity when expressed or introduced in vivo.

As used herein, the terms “wound” and “skin wound” refer to a breakdown in the protective function of the skin, such as the loss of continuity of epithelium following injury to the skin or underlying tissues caused by surgery, a cut, a laceration, a gash, a tear, a scrape, an abrasion, a scratch, a burn, chemical exposure, or as a result of a disease such as DEB (e.g., RDEB). A wound may have uniform or variable depth and morphology. A wound can be a chronic wound such as chronic open wound, a recurrent wound, a surgical wound, a blister, an ulcer, a non-healing wound, a scar, a surgical scar, or a scald. In the context of the present disclosure, wounds associated with DEB (e.g., RDEB) may be chronic open wounds and/or recurrent or acute wounds of different sizes. A chronic open wound refers to an area that does not heal and stays open for at least 12 weeks. A recurrent wound refers to an area that partially heals but then easily re-blisters. A wound may be disposed anywhere on the body, including on the torso, chest, abdomen, arms, hands, fingers, legs, feet, toes, back, buttocks, neck, or head. A wound may be discrete (e.g., having a definable size or endpoints) and/or may be continuous (e.g., extending across a swatch of skin and lacking a definable size or endpoints).

As used herein, the terms “disease” and “disorder” refer to a pathological condition of a part, organ, or system of an organism resulting from various causes, such as autoimmune defect, genetic defect, and/or environmental stress, and characterized by an identifiable group of signs or symptoms. As used herein, the term “genetic disease” may be a disease that is characterized by a protein deficiency. The protein deficiency may be caused by genetic mutations in a gene encoding such protein which causes absent, insufficient amounts of, or dysfunctional protein or may arise from the development of antibodies to the protein. A “skin disease” or “skin disorder” means a clinical condition of the skin, such as a condition that affects the skin in a subject, for example, a bullous disorder, an inflammatory skin condition, or a skin cancer. Bullous (blistering) disorders are a group of heterogeneous disorders characterized by elevated fluid-filled blistering lesions (bullae) that primarily are on the skin and mucous membranes. Bullae can be variable in sizes and the specific symptoms and severity of blistering diseases vary from one person to another, even among individuals with the same disorder. Exemplary blistering disorders include, but are not limited to, epidermolysis bullosa acquisita (EBA) and congenital epidermolysis bullosa (EB) such as dystrophic EB. EB includes a group of inherited connective tissue diseases that cause blisters on the skin and mucous membranes resulting from genetic defects. DEB is mostly caused by mutations within the COL7A1 gene, which encodes Col7 protein. To date, about 800 mutations in the COL7A1 gene have been reported. DEB has two patterns of inheritance: autosomal dominant (Dominant DEB, DDEB) and autosomal recessive (Recessive DEB, RDEB). DDEB involves reduced Col7 expression which is generally caused by glycine substitutions within the collagenous domain of the collagen alphal (VII) chain. RDEB is usually severe and caused by absence or marked reduction of Col7 expression, mostly due to premature termination codons (PTC) in the COL7A1 gene. In some embodiments, DEB (e.g., DDEB or RDEB) is diagnosed following a physical exam, laboratory test results (e.g., based on skin biopsy), review of medical history, and/or genetic testing. In some embodiments, diagnosis of DEB (e.g., DDEB or RDEB) is confirmed via genetic testing.

As used herein, the terms “patient” and “subject” refer to an individual to be treated according to the methods provided herein. A subject may be a human or a non-human mammal, such as a non-human primate, pig, goat, horse, cow, dog, cat, rat, mouse, or rabbit. A subject is preferably a human. A subject may be afflicted with a skin disorder (e.g., a genodermatosis) such as DEB (e.g., DDEB or RDEB). A subject may have previously been diagnosed with and optionally undergone a therapy for a skin disorder (e.g., a genodermatosis) such as DEB (e.g., DDEB or RDEB). A subject may be known to have a genetic mutation associated with DEB. A subject may have one or more symptoms or complications associated with DEB, such as skin wounds including blistering, itching, pain, skin erosion, scarring, skin fragility, gangrene, aplasia or hypoplasia of the skin, hypopigmentation of skin, milia, skin infections, cheilitis (e.g., inflammation of lips), dystrophic fingernails or toenails, abnormal dental enamel, pseudosyndactyly or camptodactyly of fingers or toes, finger or toe syndactyly, flexion contracture of toes, carious teeth, dysphagia (e.g., poor swallowing), furrowed or grooved tongue, esophageal stricture, laryngeal stenosis, failure to thrive, dilated cardiomyopathy, abnormal pulmonary interstitial morphology, acute constipation, hearing or visual impairment, eczema, glomerulopathy, immunologic hypersensitivity, nasolacrimal duct obstruction, anemia, chronic ear infections, corneal erosion, corneal abrasion or scarring, oral erosions, microstomia, osteopenia, ectropion, immunologic hypersensitivity, nephrotic syndrome, phimosis, renal insufficiency, urinary retention, ureteral stenosis, stroke, or squamous cell carcinoma. In some embodiments, a subject has one or more symptoms or complications associated with DEB selected from skin wounds including blistering, scarring, gangrene, aplasia or hypoplasia of the skin, hypopigmentation of skin, milia, skin infections, cheilitis (e.g., inflammation of lips), dystrophic fingernails or toenails, abnormal dental enamel, pseudosyndactyly or camptodactyly of fingers or toes, finger or toe syndactyly, flexion contracture of toes, carious teeth, dysphagia (e.g., poor swallowing), furrowed or grooved tongue, esophageal stricture, and laryngeal stenosis. In some embodiments, a subject has one or more symptoms or complications associated with DEB selected from skin wounds including blistering, scarring, aplasia or hypoplasia of the skin, cheilitis (e.g., inflammation of lips), dystrophic fingernails or toenails, pseudosyndactyly or camptodactyly of fingers or toes, and finger or toe syndactyly. A subject may be of any age or stage of development. In some embodiments, a subject is an infant or neonate. In some embodiments, a subject is less than about 24 months old, such as less than about 20, 18, 16, 14, 12, 10, 8, 6, 5, 4, 3, 2, or 1 month old. In some embodiments, a subject is at least about 24 months (e.g., 2 years) old, such as about 3, 4, 5, 6, 7, 8, 9, 10, 11, 12, 13, 14, 15, 16, 17, or 18 years old. In some embodiments, a subject is between about 2-5 years old, about 2-6 years old, about 2-8 years old, about 2-10 years old, about 6-10 years old, about 6-12 years old, about 6-18 years old, about 8-12 years old, about 8-18 years old, about 12-18 years old, about 2-13 years old, about 6-13 years old, about 13-18 years old, or any range therein. In some embodiments, a subject is at least 18 years old.

As used herein, the term “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans, or generally recognized as safe for use in, e.g., parenteral products.

As used herein, the terms “therapeutically effective dose” and “effective amount” refer to the amount of the compound that is sufficient to result in a therapeutic response. In connection with Col7 protein therapy, the terms “therapeutically effective dose” and “effective amount” may refer to the amount of the Col7 substance (e.g., rhCol7 substance), or drug product or pharmaceutical composition comprising the same, that is sufficient to result in a therapeutic response. A therapeutic response may be any response that a user (e.g., a clinician) will recognize as an effective response to the therapy. Thus, a therapeutic response will generally be an amelioration of one or more symptoms and/or complications of a disease or disorder such as DEB (e.g., DDEB or RDEB), specifically, the amount capable of inhibition, to some extent, incurrence and/or severity of skin wound; reduction in the number of skin wounds; reduction in skin lesion size; inhibition including reduction, slowing down, or complete cessation of blistering; closure of chronic open wounds; increase in the rate of healing of wounds and blisters; alleviation of skin inflammation; and enhancement of Col7 incorporation to the epidermis-dermis junctions.

As used herein, “treating.” “treat,” and “treatment” refer to administering to a subject having a disease or disorder, or a symptom or complication thereof, a pharmaceutical composition, such that at least one symptom or complication of the disease or disorder is reversed, cured, alleviated, ameliorated, eliminated, delayed, or decreased. Treating EB (e.g., DEB such as DDEB or RDEB) in a subject refers to administering to the subject having an EB a pharmaceutical composition (e.g., a pharmaceutical composition comprising an rhCol7 substance, as described herein) such that at least one symptom or complication of the EB is reversed, cured, alleviated, ameliorated, eliminated, delayed, or decreased. The symptoms or complications of an EB disease that may be targeted for treatment include, but are not limited to, skin wounds including blistering; itching; pain; lesions (e.g., rectal, anal, urethral lesions and/or mucosal lesions and/or lesions of squamous epithelial tissue); skin erosion; scarring; skin fragility; gangrene; aplasia or hypoplasia of the skin; hypopigmentation of skin; milia; skin infections; cheilitis (e.g., inflammation of lips); dystrophic fingernails or toenails; lesions of the gastrointestinal tract; contractures, e.g., flexion contractures (e.g., of the extremities); pseudosyndactyly or camptodactyly of the hands or feet; carcinoma (e.g., squamous cell carcinoma); bulla formation; nail and/or teeth deformities; constricted esophagus; laryngeal stenosis; furrowed or grooved tongue; abnormal dental enamel; carious teeth; dysphagia (e.g., poor swallowing); eye disorders; hearing or visual impairment; anemia; malnutrition;secondary skin infection; sepsis; hoarse voice; urethral stenosis; phimosis; corneal scarring or erosion; corneal abrasion; oral erosions; microstomia; osteopenia; malabsorption; dilated cardiomyopathy; abnormal pulmonary interstitial morphology; acute constipation; eczema; glomerulopathy; immunologic hypersensitivity; nasolacrimal duct obstruction; chronic car infections; ectropion; immunologic hypersensitivity; nephrotic syndrome, phimosis; renal insufficiency; urinary retention; ureteral stenosis; stroke; and failure to thrive.

As used herein, the terms “preventing,” “prevent,” and “prevention” refer to administering to a subject a pharmaceutical composition, e.g., prior to a clinical manifestation of an unwanted condition (e.g., disease or other unwanted state of the host animal) so that it protects the host against developing the unwanted condition or a symptom or condition thereof. “Preventing” a disease may also be referred to as “prophylaxis” or “prophylactic treatment.” In the context of the present disclosure, one or more symptoms or complications associated with EB, such as skin blistering or scarring, may be prevented by administration of a pharmaceutical composition (e.g., a pharmaceutical composition comprising an rhCol7 substance). Scarring in a subject with EB may result in one or more of the following symptoms: contractures, e.g., flexion contractures (e.g., of the extremities); pseudosyndactyly of the hands or feet; carcinoma (e.g., squamous cell carcinoma); rectal lesions; mucosal lesions; bulla formation; bulla formation post manual trauma; nail or teeth deformities; constricted esophagus; eye disorders, anemia, malnutrition; secondary skin infection; sepsis; hoarse voice; urethral stenosis; phimosis; corneal scarring; malabsorption; and failure to thrive. In some embodiments, a symptom or complication of an EB disease (e.g., DEB such as DDEB or RDEB) that may be prevented by administration of a pharmaceutical composition (e.g., a pharmaceutical composition comprising an rhCol7 substance) may be, for example, skin wounds including blistering; itching; pain; lesions (e.g., rectal, anal, urethral lesions and/or mucosal lesions and/or lesions of squamous epithelial tissue); skin erosion; scarring; skin fragility; gangrene; aplasia or hypoplasia of the skin; hypopigmentation of skin; milia; skin infections; cheilitis (e.g., inflammation of lips); dystrophic fingernails or toenails; lesions of the gastrointestinal tract; contractures, e.g., flexion contractures (e.g., of the extremities); pseudosyndactyly or camptodactyly of the hands or fect; carcinoma (e.g., squamous cell carcinoma); bulla formation; nail and/or teeth deformities; constricted esophagus; laryngeal stenosis; furrowed or grooved tongue; abnormal dental enamel; carious teeth; dysphagia (e.g., poor swallowing); eye disorders; hearing or visual impairment; anemia; malnutrition; secondary skin infection; sepsis; hoarse voice; urethral stenosis; phimosis; corneal scarring or erosion; corneal abrasion; oral erosions; microstomia; osteopenia; malabsorption; dilated cardiomyopathy; abnormal pulmonary interstitial morphology; acute constipation; eczema; glomerulopathy; immunologic hypersensitivity; nasolacrimal duct obstruction; chronic ear infections; ectropion; immunologic hypersensitivity; nephrotic syndrome, phimosis; renal insufficiency; urinary retention; ureteral stenosis; stroke; and failure to thrive.

As used herein, the terms “improve,” “increase,” “reduce,” and “decrease,” and grammatical equivalents thereof, indicate modulation of values relative to a baseline measurement, such as a measurement in the same individual prior to initiation of the treatment described herein, or a measurement in a control subject (or multiple control subjects) in the absence of the treatment described herein.

As used herein, the term “dosing interval” refers to the amount of time that elapses between multiple doses being administered to a subject (e.g., a human patient). The dosing interval may alternately be referred to herein as the dosing frequency (e.g., the frequency at which a dose is administered to a subject). In some embodiments, a single dosing interval is used for the duration of therapy with a Col7 substance (e.g., rhCol7 substance). Such a dosing interval may be considered to be “fixed” within a given dosing regimen. In some embodiments, multiple dosing intervals are used over the course of therapy with a Col7 substance (e.g., rhCol7 substance). In some embodiments, a dosing interval is less than about one day, about one day, about 2 days, about 3 days, about 4 days, about 5 days, about 6 days, about one week, about two weeks, about three weeks, about one month, about two months, about three months, about six months, or longer. In some embodiments, a dosing interval is an individualized interval that is determined for a given subject (e.g., human patient) based on, e.g., pharmacokinetic/toxicokinetic (PK/TK) data or other information about that subject, such as age, weight, height, body mass index, disease severity, comorbidities, race, ethnicity, national origin, genotype, and/or prior response to a Col7 replacement therapy. An individualized dosing interval may be the same dosing interval as for a fixed dosing interval (e.g., under another therapeutic scheme or for another subject) or may differ. A therapeutic regimen comprising administration of a Col7 substance (e.g., rhCol7 substance) to a subject may include multiple dosing regimens, one or more of which may comprise a fixed dosing interval and one or more of which may comprise an individualized dosing interval, as described herein. In some embodiments, a therapeutic regimen comprising administration of a Col7 substance (e.g., rhCol7 substance) to a subject includes a first dosing regimen comprising administration of the Col7 substance at a first dosing interval (e.g., at a first frequency), and a second dosing regimen comprising administration of the Col7 substance at a second dosing interval (e.g., at a second frequency), where the first dosing interval and the second dosing interval are both fixed dosing intervals with different dosing frequencies, and where the first dosing regimen is performed before the second dosing regimen. In some embodiments, a therapeutic regimen comprising administration of a Col7 substance (e.g., rhCol7 substance) to a subject includes a first dosing regimen comprising administration of the Col7 substance at a first dosing interval (e.g., at a first frequency), and a second dosing regimen comprising administration of the Col7 substance at a second dosing interval (e.g., at a second frequency), where the first dosing interval is a fixed dosing interval and the second dosing regimen is an individualized dosing interval, and where the first dosing regimen is performed before the second dosing regimen. In other embodiments, a therapeutic regimen comprising administration of a Col7 substance (e.g., rhCol7 substance) to a subject includes a first dosing regimen comprising administration of the Col7 substance at a first dosing interval, and a second dosing regimen comprising administration of the Col7 substance at a second dosing interval, where the second dosing interval is a fixed dosing interval and the first dosing regimen is an individualized dosing interval, and where the first dosing regimen is performed before the second dosing regimen. In some embodiments, a dosing regimen includes administering a dose of a drug substance to a subject (e.g., a human patient) more than once per day, about once per day, about every 2 days, about every 3 days, about every 4 days, about every 5 days, about every 6 days, about once a week, about every other week, about every three weeks, about once a month, about every two months, about every three months, about every six months, or less frequently.

As used herein, the term “clinical parameter” refers to a parameter of a clinical measurement. A clinical parameter may relate to a complication and/or symptom associated with DEB such as a size and number of skin wounds, duration of chronic open wounds, the rate of healing of wounds and blisters, etc. A clinical parameter may be assessed by visual or physical inspection, a biopsy, and/or a biochemical assay, such as an assay of urine, blood, or plasma.

### DRUG SUBSTANCE AND COMPOSITIONS

### Active Substance: rhCol7

The present disclosure relates to intravenous rhCol7 replacement therapy to treat cutaneous and systemic manifestations of dystrophic epidermolysis bullosa (DEB), particularly recessive DEB (RDEB), in subjects. The active drug substance for systemically treating a clinical condition of DEB, is recombinant human collagen 7 protein (rhCol7). As described herein, anchoring fibrils formed by assembly of Col7 dimers facilitate the attachment of the epidermis to the underlying dermis. Pathogenic mutations in the gene encoding collagen alpha-1 (Type VII) chain polypeptide (COL7A1) lead to abnormal, decreased, or a complete absence of anchoring fibrils in the skin, resulting in DEB. This lack of normal functioning anchoring fibrils is responsible for cutaneous and non-cutaneous symptoms including poor epidermal-dermal adherence, pronounced skin fragility, and severe blistering in the skin and mucosa in DEB patients.

The direct mechanistic link between the genetic defect or absence of Col7 in anchoring fibrils and DEB makes appropriate introducing native Col7 or functional equivalent, such as a recombinant Col7 (e.g., rhCol7) for treating DEB patients. Previous nonclinical data demonstrate that intravenous administration of rhCol7 distributes to and is selectively retained at the dermal-epidermal BMZ, forms normal anchoring fibrils, reverses dermal-epidermal separation by providing stability to dermal-epidermal adhesion at the lamina densa/upper papillary dermis interface, and results in a statistically significant improvement in survival in a mouse model of DEB.

Specifically, RDEB is a devastating systemic disease with no effective therapy. Current therapies in development are unlikely to address significant gastrointestinal/genitourinary (GI/GU) morbidity, corneal, and oral manifestations of DEB, or development of squamous cell carcinoma (SCC), the major source of mortality. No other systemic disease modifying approaches are in development. Recombinant collagen 7 has demonstrated a reasonably favorable toxicological profile, including all observations related to immune mediated response to “foreign” protein. Therefore, it is reasonable that intravenous delivery of rhCol7 will restore functional Col7 to the dermal-epidermal BMZ. The restoration may promote the assembly of normal anchoring fibrils, providing stability to dermal-epidermal adhesion at the lamina densa/upper papillary dermis interface and correct blistering abnormalities and complications in patients with DEB.

In accordance with the present disclosure, the active ingredient of the drug substance is a recombinant human alpha-1 chain homo-trimer of type VII collagen. In some embodiments, the active drug substance is a recombinant form of the Col7 protein produced in engineered cells that are transfected with an expression plasmid encoding the full-length human collagen VII sequence.

In some embodiments, the alpha-1 chain (Type VII collagen) polypeptide comprises an amino acid sequence of SEQ ID NO: 1. In some embodiments, the alpha-1 chain (Type VII collagen) polypeptide consists of an amino acid sequence of SEQ ID NO: 1. In some embodiments, the alpha-1 (Type VII collagen) chain polypeptide may comprise an amino acid sequence that is about 75%, 80%, 85%, 86%, 87%, 88%, 89%, 90%, 91%, 92%, 93%, 94%, 95%, 96%, 97%, 98%, 99%, or 100% identical to the sequence of SEQ ID NO: 1. In some embodiments, the alpha-1 chain polypeptide may be encoded by a polynucleotide comprising a nucleic acid sequence of SEQ ID NO: 2. The rhCol7 encoding polynucleotide sequence may be about 75%, 80%, 85%, 86%, 875, 88%, 89%, 90%, 91%, 92%, 93%, 94%, 95%, 96%, 97%, 98%, 99% or 100% identical to the sequence of SEQ ID NO: 2. In some embodiments, the rhCol7 encoding polynucleotide sequence may be codon optimized. Codon optimization may serve to increase glycine content of the coding nucleotide sequence. In some embodiments, the rhCol7 encoding polynucleotide sequence comprises one or more modifications. In some embodiments, the one or more modifications increase the stability of the encoding polynucleotide.

In some embodiments, the rhCol7 substance comprises a plurality of recombinant Col7 alpha-1 chain polypeptides, and/or functional variants thereof.

In some embodiments, the rhCol7 substance is produced in a mammalian cell line engineered to express or manufacture rhCol7. In some embodiments, the rhCol7 substance is produced using genetically modified mammalian cells. As used herein, the term “genetically modified” refers to cells that express a particular gene product following introduction of a nucleic acid comprising a coding sequence which encodes the gene product. Introduction of the nucleic acid may be accomplished by any method known in the art including gene targeting and homologous recombination. As used herein, the term also includes cells that have been engineered to express or overexpress an endogenous gene or gene product not normally expressed by such cell. In some embodiments, the rhCol7 substance is produced in mammalian cells such as CHO cells. The CHO cells may be modified to comprise a polynucleotide encoding the alpha-1 chain (Type VII) and at least one polynucleotide encoding a protein that can increase the expression of the Col7 alpha-1 chain in the host cells, e.g., prolidase, prolyl 4-hydroxylase, and/or heat shock protein 47 (HSP 47). The genetically engineered cell line used to manufacture rhCol7 may be referred to as a Master Cell Bank (MCB). In one preferred embodiment, the rhCol7 production cells may be derived from a Chinese hamster ovary CHO-K1 host cell line which is transfected with plasmids encoding rhCol7 as well as the prolyl hydroxylase alpha and beta subunits to promote proline hydroxylation (P4H), a post-translational modification that stabilizes the rhCol7 structure. In some embodiments, the rhCol7 substance is produced by the master cells co-expressing rhCol7 and prolidase, as described in U.S. Pat. No. 9,676,837, the contents of which are incorporated herein by reference in their entirety. In some embodiments, the rhCol7 substance is produced by the master cells co-expressing rhCol7 and prolyl 4-hydroxylase, as described in International Patent Publication No. WO2020025129, the contents of which are incorporated herein by reference in their entirety. The rhCol7 substance may be produced in cell culture and purified using a series of chromatographic and filtration steps, then processed into drug product. Each lot of drug substance and drug product may be evaluated for quality and stability.

rhCol7 expressing MCB cells may be progressively expanded in vessels of increasing size using batch and/or perfusion culture to reach a high cell density in the production bioreactors. Intermediate harvests from the production bioreactors may be collected daily and clarified. The clarified harvests may be concentrated, frozen, and later pooled for purification to generate the rhCol7 substance. The clarified, concentrated harvest pools may be purified using multiple chromatography processes. In some cases, three chromatography steps including mixed mode, cation exchange, and hydrophobic interaction chromatography unit operations may be used. For example, the recombinant products may be purified through purification processes including but not limited to ultrafiltration/diafiltration (UF/DF) membrane capture, CaptoCore, SP Sepharose, and polypropylene glycol (PPG) chromatography steps. The purification process may also include one or more viral reduction processes, such as three orthogonal viral reduction steps including UV-C and detergent (Triton X-100) viral inactivation steps as well as viral filtration (e.g., Asahi Planova 35N). The processed rhCol7 substance is preferably free of viruses. The purified product may be concentrated and filtered (e.g., diafiltered) into a formulation buffer. Polysorbate 20 may be added to generate the final drug substance composition.

In some embodiments, a drug substance includes one or more salts, surfactants, buffers, amino acids (e.g., non-essential amino acids), cryoprotectants, and/or chelating agents. In some embodiments, a drug substance includes a non-essential amino acid, which non-essential amino acid is preferably arginine. In some embodiments, a drug substance includes a phosphate buffer, which phosphate buffer is preferably sodium phosphate. In some embodiments, a drug substance includes a chelating agent, which chelating agent is preferably sodium citrate. In some embodiments, a drug substance includes a cryoprotectant, which cryoprotectant is preferably a sugar such as sucrose. In some embodiments, a drug substance includes sodium chloride. In some embodiments, a drug substance includes a surfactant, a buffer, a non-essential amino acid, a cryoprotectant, and a chelating agent. In some embodiments, a drug substance includes sodium phosphate, sodium citrate, arginine (e.g., L-arginine), sucrose, sodium chloride, and polysorbate 20. In some embodiments, a drug substance includes 1.2 mg/mL recombinant rCol7 in 10 mM sodium phosphate, 5 mM sodium citrate, 100 mM L-arginine, 1.7% sucrose (w/v), 70 mM sodium chloride, and 0.05% (v/v) polysorbate 20, pH 7.2. In some embodiments, the bulk drug substance is 0.2 micrometer (um) filtered, filled into sterile containers, and stored at ≤−60° C. for the drug production.

The rhCol7 substance is preferably free of microbial and fungal adventitious agents. The structural and functional characteristics of the rhCol7 substances may be evaluated and compared with native Col7 protein.

In some embodiments, rhCol7 substance is a systemic protein therapy to be delivered via the intravenous route.

### Formulations and Drug Products

The drug products of the present disclosure contain at least one active ingredient, recombinant human Col7 (rhCol7). The rhCol7 substance may be produced using MCB cells as discussed herein. The rhCol7 substance may be included in the drug product at a target protein concentration of about 0.01 milligrams per milliliter (mg/mL) to 100 mg/mL, about 0.1 mg/mL to 100 mg/mL, about 0.5 mg/mL to 100 mg/mL, about 1 mg/mL to 100 mg/mL. about 10 mg/mL to 100 mg/mL, about 0.01 mg/mL to 50 mg/mL, about 0.1 mg/mL to 50 mg/mL, about 0.5 mg/mL to 50 mg/mL, about 1 mg/mL to 50 mg/mL, about 0.01 mg/mL to 20 mg/mL, about 0.1 mg/mL to 20 mg/mL, about 0.5 mg/mL to 20 mg/mL, about 1 mg/mL to 20 mg/mL, about 10 mg/mL to 50 mg/mL, about 0.01 mg/mL to 20 mg/mL, about 0.1 mg/mL to 20 mg/mL, about 0.5 mg/mL to 20 mg/mL, about 1 mg/mL to 20 mg/mL, about 0.01 mg/mL to 10 mg/mL, about 0.1 mg/mL to 10 mg/mL, about 0.5 mg/mL to 10 mg/mL, or about 1.0 mg/mL to 10 mg/mL. In some examples, the rhCol7 substance is included in the drug product at a target protein concentration of about 0.1 mg/mL, about 0.2 mg/mL, about 0.3 mg/mL, about 0.4 mg/mL, 0.5 mg/mL, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3.0 mg/mL, about 3.5 mg/mL, about 4.0 mg/mL, about 4.5 mg/mL, about 5.0 mg/mL, about 5.5 mg/mL, about 6.0 mg/mL, about 6.5 mg/mL, about 7.0 mg/mL, about 7.5 mg/mL, about 8.0 mg/mL, about 8.5 mg/mL, about 9.0 mg/mL, about 9.5 mg/mL, about 10 mg/mL, about 15 mg/mL, or about 20 mg/mL. In some embodiments, the drug product contains recombinant human collagen VII (rhCol7) at a target concentration of 1.2 mg/mL.

In some embodiments, the rhCol7 drug product comprises at least one pharmaceutically acceptable excipient, including those commonly used in parenteral biopharmaceutical formulations. In some examples, the excipients added in the present rhCol7 drug products include, but are not limited to, sodium phosphate dibasic, sodium citrate dihydrate, sodium chloride, L-arginine HCl, sucrose, and polysorbate 20.

Buffers may be selected from phosphate buffered saline (PBS), or other suitable buffers. The buffer is suitable for the target pH range, excipients, and surfactants. In some examples, the rhCol7 drug product may have a pH ranging from about pH 6.8 to about pH 7.5. For example, the pH of the rhCol7 drug product may be about 6.6, about 6.7, about 6.8, about 6.9, about 7.0, about 7.1, about 7.2, about 7.3, about 7.4, or about 7.5.

The rhCol7 drug products provided herein may have significantly improved solubility and reduced viscosity at higher protein concentrations. In some embodiments, agents are added to increase the stability of the drug substance of the product, including but not limited to L-arginine (e.g., 100 millimolar (mM) L-arginine), and sucrose.

In some embodiments, the rhCol7 formulation comprises about 5-200 mM, about 10-150 mM, about 20-150 mM, about 50-150 mM about 50-100 mM, about 75-100 mM, or about 100 mM L-arginine.

In some embodiments, the rhCol7 formulation comprises about 10-100 mM, about 75-100 mM, about 50-100 mM, about 15-75 mM, about 35-75 mM, about 50-75 mM, or about 50 mM sucrose.

In some embodiments, a surfactant is included in the rhCol7 drug product. In some embodiments, the surfactant is selected from the group consisting of polysorbate 20, polysorbate 40, polysorbate 60, polysorbate 80, and a combination thereof. In some embodiments, the surfactant is polysorbate 20. In some embodiments, the polysorbate 20 is present at a concentration ranging from approximately 0.01% to 0.2%, such as from about 0.01% to 0.15%, about 0.01% to 0.1%, about 0.01% to 0.08%, or about 0.01% to 0.06%. In some embodiments, the drug product includes 0.05% polysorbate 20.

The present drug products are formulated to provide formulation conditions that allow for acceptable freeze/thaw (F/T) performance and improved stability profiles to support storage at frozen conditions, e.g., either −20° C. or at ≤−60° C.

In some embodiments, the present rhCol7 drug product is ready-to-fill. Therefore, all drug product excipients are incorporated at the drug substance (i.e., rhCol7 production) manufacturing stages, for example, the MCB cell culture and cultural media harvest and purification processes; there are no additional excipients added during the rhCol7 drug product manufacturing process.

In some embodiments, the rhCol7 drug product of the present disclosure is sterile. In some embodiments, the rhCol7 drug product of the present disclosure is free of preservatives.

In some embodiments, the rhCol7 drug product is manufactured using aseptic processing techniques. The drug product may be sterile-filtered into sterile, depyrogenated glass vials and stoppered and sealed in an ISO 5/Class 100/Grade A area. In process testing may be performed to ensure the sterility through the entire manufacturing process. The in-process testing may include integrity testing of bioburden reduction (0.2 micrometer (μm) filtration) pre-and/or post-use, bioburden testing prior to sterile filtration, and/or endotoxin testing. Environmental monitoring including particulates and microbial monitoring, may also be performed throughout the drug product manufacturing process.

In some embodiments, the rhCol7 drug product is directly prepared/manufactured from the rhCol7 substance from MCB cell cultures. In some embodiments, the frozen rhCol7substance (e.g., cultural media harvested from the MCB culture) is removed from storage, thawed (e.g., in temperature-controlled water bath), pooled, and mixed, and subsequently subjected to a bioburden reduction filtration through a 0.5/0.2 μm membrane filter unit. Sterile filtration of the pooled bulk drug product may be performed using two consecutive 0.5/0.2 μm sterile filter units. The drug product may be aseptically filled into sterile vials (e.g., 10R glass vials with stopper and flip cap), which vials may then be stoppered and crimped. The filled vials may be 100% visually inspected and defect vials are rejected. All acceptable vials are labelled and packaged at room temperature and then stored at −60° C.

In some embodiments, a drug formulation comprising recombinant human Col7(rhCol7) substance is in the form of a clear to slightly opalescent, colorless solution. As a non-limiting example, the rhCol7 drug product is PTR-01, in which the rhCol7 substance is formulated to a target concentration of 1.2 mg/mL recombinant Col7 in 10 mM sodium phosphate, 5 mM sodium citrate, 100 mM L-arginine, 1.7% sucrose (w/v), 70 mM sodium chloride, and 0.05% (v/v) polysorbate 20 at pH 7.2. Additional examples of rhCol7 drug products are described in International Patent Publication No. WO2017112757, which is herein incorporated by reference in its entirety.

In some embodiments, the rhCol7 substance is formulated for systemic administration. In some embodiments, the rhCol7 substance is formulated for intravenous administration such as intravenous infusion. In some embodiments, the rhCol7 drug product is an injectable aqueous solution.

In some embodiments, an rhCol7 composition is supplied as a sterile, preservative-free, frozen liquid in 10 mL clear, borosilicate glass vials (10R. Type I glass). Each vial may have a 20 millimeter (mm) grey butyl rubber stopper and a 20 mm aluminum flip cap. Each vial may be filled with a target volume of 5.5 milliliter (mL) to ensure a withdrawal volume of at least 5.0 mL (i.e., a target weight of 6.0 mg dose) from each vial. Each vial may be for single use.

In some embodiments, the rhCol7 drug product is stored at ≤−60° C. and stable for at least 24 months at ≤−60° C., or at least 32 months at ≤−60° C., or at least 36 months at ≤−60° C., at least 42 months at ≤−60° C., or at least 48 months at ≤−60° C. In other embodiments, the rhCol7 drug product may be stored at −20±5° C. and stable for at least 12 months at −20±5° C., or at least 18 months at −20±5° C., or at least 24 months at −20±5° C., or at least 30 months at −20±5° C., or at least 36 months at −20±5° C. In other embodiments, the rhCol7 drug product may be stored at 5±3° C. and is stable for up to one month.

The present drug product may be clinically prepared as dosing solutions before intravenous injection or infusion. During clinical use, dosing solutions may be prepared at fixed volumes in sterile saline (0.9% sodium chloride) with a final dosing solution volume of about 10-1000 mL, about 20-1000 mL, about 40-1000 mL, about 50-1000 mL, about 100-1000 mL, about 200-1000 mL, about 300-1000 mL, about 400-1000 mL, about 500-1000 mL, about 10-500 mL, about 20-500 mL, about 40-500 mL, about 50-500 mL, about 100-500 mL, about 200-500 mL, about 300-500 mL, about 400-500 mL, about 10-300 mL, about 20-300 mL, about 40-300 mL, about 50-300 mL, about 100-300 mL, or about 200-300 mL, such as about 10 mL, 20 mL, 30 mL, 40 mL, 50 mL, 60 mL, 70 mL, 80 mL, 90 mL, 100 mL, 150 mL, 200 mL, 250 mL. 300 mL, 350 mL, 400 mL, 450 mL, 500 mL, 600 mL, 700 mL, 800 mL, 900 mL, or 1000 mL. The dosing solution may be administered intravenously with dose preparation at the clinical or hospital center pharmacy. In some examples, the dosing solution may be prepared in an IV bag such as a PVC IV bag. The dosing solution may be stable for up to 24 hours of storage at ambient room temperature.

### DISEASES AND APPLICATIONS

The rCol7 substance, drug products, and compositions and methods of the present disclosure can provide prophylactic, palliative, or therapeutic relief of a wound, disorder, or disease of the skin in a subject, specifically a skin disorder caused by deficiency in Col7, e.g., Dystrophic Epidermolysis Bullosa (DEB), including the two main subtypes: recessive dystrophic epidermolysis bullosa (RDEB) and dominant dystrophic epidermolysis bullosa (DDEB). Other skin disorders may include, but are not limited to, skin cancer, psoriasis, lichen planus, lupus, rosacea, eczema, cutaneous candidiasis, cellulitis, impetigo, decubitus ulcers, crysipelas, ichthyosis vulgaris, dermatomyositis, acrodermatitis, stasis dermatitis, burns, and Netherton syndrome.

In some embodiments, Recessive Dystrophic Epidermolysis Bullosa (RDEB) includes all subtypes such as RDEB-generalized severe (formerly known as the Hallopeau-Siemens variant), RDEB-generalized other, RDEB-inversa, RDEB-pretibial, RDEB-pruriginosa, RDE-centripetalis, and RDEB-bullous dermolysis of the newborn (Soro L. et al., J. Anesthel Dermatol., 2015, 8 (5): 41-46). In other embodiments, dominant dystrophic epidermolysis bullosa (DDEB) includes all subtypes such as DDEB-generalized, DDEB-acral, DDEB-pretibial, DDEB-pruriginosa, DDEB-nails only, and DDEB-bullous dermolysis of the newborn (Fine et al., J. Am. Acad. Dermatol. 2008; 58931-950).

The rhCol7 substance, drug products, and compositions provided herein can provide therapeutic relief, including alleviating, preventing, or preventing the progression of one or more complications and symptoms of DEB, especially RDEB, including cutaneous and systemic manifestations in patients.

In accordance with the present disclosure, use of the rhCol7 substance, drug products, and compositions provided herein for therapeutically treating a subject with RDEB by systemic administration of an effective amount of the rhCol7 substance is capable of enhancing, increasing, augmenting, and/or supplementing the levels of a collagen alpha-1 (type VII) chain polypeptide, particularly the levels of Col7 present at the BMZ of the skin.

In some embodiments, the patient to be treated is diagnosed with RDEB. The patient may be diagnosed with RDEB by genetical diagnosis for mutations in the COL7A1 gene.

In some embodiments, the subject is an adult. For example, the subject may be at least 18 years of age. In some embodiments, the subject is an elderly subject. For example, the subject may be at least 55 years of age, at least 60 years of age, at least 65 years of age, at least 70 years of age, at least 75 years of age, or at least 80 years of age. In some embodiments, the subject is a pediatric subject that is less than 18 years of age. In some embodiments, a subject is less than about 24 months old, such as less than about 20, 18, 16, 14, 12, 10, 8, 6, 5, 4, 3, 2, or 1 month old. In some embodiments, a subject is at least about 24 months (e.g., 2 years) old, such as about 3, 4, 5, 6, 7, 8, 9, 10, 11, 12, 13, 14, 15, 16, 17, or 18 years old. In some embodiments, a subject is between about 2-5 years old, about 2-6 years old, about 2-8 years old, about 2-10 years old, about 2-12 years old, about 6-10 years old, about 6-12 years old, about 6-18 years old, about 8-12 years old, about 8-18 years old, about 12-18 years old, about 2-13 years old, about 6-13 years old, about 13-18 years old, or any range therein. In some embodiments, a subject is at least 18 years old. In some embodiments, a subject is an infant or neonate (e.g., newborn). In some embodiments, a subject is less than about 24 months old, such as less than about 20, 18, 16, 14, 12, 10, 8, 6, 5, 4, 3, 2, or 1 month old. In some embodiments, a subject is less than about 1 year old. In some embodiments, the pediatric subject is diagnosed with RDEB. In some embodiments, the newborn subject is diagnosed with RDEB.

In some embodiments, the subject to be treated has chronic wounds such as chronic open wounds and recurrent wounds. In some embodiments, the subject has chronic open wounds. In some embodiment, the subject has blisters. In other embodiments, the subject has recurrent wounds. In some embodiments, the subject has asplasia or hypoplasia of the skin. In some embodiments, the subject has pseudosyndactyly or camptodactyly of fingers or toes, and finger or toe syndactyly. In some embodiments, the subject has cheilitis. In some embodiments, the subject has dysphagia, furrowed or grooved tongue, esophageal stricture, and/or laryngeal stenosis. In some embodiments, the subject has chronic or recurrent wounds, blisters, aplasia of the skin, hypoplasia of the skin, pseudosyndactyly or camptodactyly of fingers or toes, finger or toc syndactyly, cheilitis, dysphagia, furrowed or grooved tongue, esophageal stricture, laryngeal stenosis, or a combination thereof.

In some embodiments, the subject is suspected of having a mutation of the COL7A1 gene. In some embodiments, the subject has undergone genetic testing to confirm a COL7A1 mutation.

METHODS OF TREATING EPIDERMOLYSIS BULLOSA

The present disclosure provides methods of treating skin disorders such as dystrophic epidermolysis bullosa (DEB, such as RDEB), or symptoms or complications thereof, using Col7 substances (e.g., rhCol7 substances). Treating a subject according to a method provided herein may result in a change such as an improvement in the condition of the subject, as measured by visual inspection, physical exam, laboratory exam, self-reporting by the subject, or any other useful measure. Treating a subject according to a method provided herein may result in amelioration, alleviation, reduction, improvement, delay of onset, delay of progression, elimination, and/or curing of one or more symptoms or complications of a skin disorder such as DEB (e.g., RDEB). The present disclosure also provides methods of ameliorating, alleviating, reducing, improving, delaying onset of, delaying progression of, eliminating, and/or curing one or more symptoms or complications of a skin disorder such as DEB. Symptoms and complications of DEB (e.g., RDEB) may include, for example, cutaneous and systemic symptoms. Symptoms and complications of DEB may include, for example, skin wounds including blistering, itching, pain, skin erosion, scarring, skin fragility, gangrene, aplasia or hypoplasia of the skin, hypopigmentation of skin, milia, skin infections, cheilitis (e.g., inflammation of lips), dystrophic fingernails or toenails, abnormal dental enamel, pseudosyndactyly or camptodactyly of fingers or toes, finger or toe syndactyly, flexion contracture of toes, carious teeth, pysphagia (e.g., poor swallowing), furrowed or grooved tongue, esophageal stricture, laryngeal stenosis, failure to thrive, dilated cardiomyopathy, abnormal pulmonary interstitial morphology, acute constipation, hearing or visual impairment, eczema, glomerulopathy, immunologic hypersensitivity, nasolacrimal duct obstruction, anemia, chronic ear infections, corneal erosion, corneal abrasion and scarring, oral erosions, microstomia, osteopenia, ectropion, immunologic hypersensitivity, nephrotic syndrome, phimosis, renal insufficiency, urinary retention, ureteral stenosis, stroke, or squamous cell carcinoma.

In some embodiments, a method of treating DEB (e.g., RDEB), or a symptom or complication thereof, in a subject comprises administering to the subject a Col7 substance (e.g., rhCol7 substance). In some embodiments, the subject has been diagnosed with DEB (e.g., RDEB) or is at a risk of developing a severe skin disorder such as DEB.

In some embodiments, a method of treating DEB (e.g., RDEB), or a symptom or complication thereof, in a subject comprises administering to the subject a therapeutically effective amount of a rhCol7 drug product by systemic (e.g., intravenous) administration. In some embodiments, the rhCol7 drug product comprises recombinant human Col7 alpha-1 chain (Type VII) as active drug substance formulated in an injectable aqueous solution. In some embodiments, the rhCol7 drug product is PTR-01.

In some embodiments, prior to receiving the rhCol7 protein therapy, a subject with DEB is prescreened and identified as suitable for treatment with the present rhCol7 protein therapy. Prescreening methods may include analyzing the genetic mutations in the COL7A1 gene and/or measuring the Col7 deficiency at the dermal-epidermal junction by immunofluorescence (IF) staining. The subject may be further evaluated for the presence of antibodies specific to Col7, e.g., Col7 neutralizing antibodies.

In some embodiments, the method further includes analyzing the genetic mutations in the COL7A1 gene in the subject to be treated (e.g., prior to initial administration of rhCol7 protein therapy). In some embodiments, the method includes selecting a subject for such treatment on the basis that the subject carries a mutation in the COL7A1 gene and is consistent with a recessive inheritance pattern (i.e., RDEB). In one aspect, the present disclosure provides a method for treating a subject with RDEB, comprising: i) identifying a subject having a COL7A1 mutation consistent with a recessive inheritance pattern; and ii) administering a pharmaceutically effective amount of an rhCol7 drug product to the subject intravenously.

One or more parameters (e.g., complications and clinical symptoms associated with RDEB) may be assessed following administration of a rhCol7 protein therapy. In some embodiments, the one or more parameters are measured at the conclusion of a defined dosing period, such as an initial dosing interval. In some embodiments, the one or more parameters are measured at a defined duration of treatment, such as after 1 month, 3 months, 6 months, 1 year, 2 years, or more. In some embodiments, the one or more parameters are compared to assessments of the one or more parameters prior to initiation of rhCol7 protein therapy. Assessment of the one or more parameters may be used to determine one or more aspects of the dosing regimen such as the dosage, dosing frequency, or duration. In some embodiments, the method further comprises performing or reviewing a serial photographic quantitative assessment of the wounds and blisters of the subject being treated. Such an assessment may evaluate, for example, wound surface area (WSA), percent wound healing, and/or time to re-blistering and may be beneficial for subjects with RDEB given the chronic and dynamic nature of wounds in RDEB patients (Solis D. et al., J Invest Dermatol, 2018, 138, Supplement: 97; doi.org/10.1016/j.jid.2018.03.580).

In some embodiments, rhCol7 treatment increases wound healing (e.g., the frequency and/or completeness or sustainability of wound healing) in a subject with RDEB. In some embodiments, a wound of a subject heals faster while the subject is under rhCol7 than it would otherwise heal or had previously healed prior to commencement of treatment. In some embodiments, a wound of a subject heals more completely and/or more sustainably while the subject is under rhCol7 that it would otherwise heal or had previously healed prior to commencement of treatment. The wound may be a chronic open wound, and/or a recurrent wound. In some embodiments, the wound healing is sustained. In some embodiments, a majority of visible wounds or lesions are improved. In some embodiments, the improvement in a wound lesion is at least 2 levels according to a 7-point Global Impression of Change instrument (Table 1). In some embodiments, the improvement in a wound or lesion is assessed via visual inspection by a physician and/or caregiver. In some embodiments, the improvement in a wound or lesion is assessed via patient interview or self-evaluation.

In some embodiments, rhCol7 treatment increases the deposition of rCol7 into the DEJ of the skin. The deposition of collagen 7 protein to the DEJ can induce formation of anchoring fibrils. Accordingly, the present disclosure further provides a method of inducing formation of anchoring fibrils in the skin, comprising administering rhCol7 to the subject (e.g., as described herein).

In some embodiments, rCol7 treatment can reduce the wound surface area in a subject, including wound surface area of target lesions and/or total body wound surface area. The wound surface area can be measured by imaging the wound areas. In some embodiments, a skin wound reduces in size after rCol7 therapy. The size of a skin wound may be reduced by, for example, at least about 10%, 20%, 30%, 40%, 50%, 60%, 70%, 80%, 90%, or more. In some embodiments, a skin wound completely heals or closes in response to rCol7 therapy (e.g., the size is reduced by about 100%). In some embodiments, the number or abundance of skin wounds and lesions is reduced in a subject following rCol7 therapy.

In some embodiments, rCol7 treatment improves skin integrity, as assessed by, e.g., a decrease in suction blister time and/or location and/or an increase in time to re-blistering.

In some embodiments, rCol7 treatment reduces pain associated with a skin wound, such as severity of pain and/or impact of pain on quality of life, which may be evaluated by the modified PROMIS subscales and iscorEB.

In some embodiments, rCol7 treatment reduces itch or irritation associated with skin wounds such as severity of itch or irritation and/or impact of itch or irritation on quality of life, which may be evaluated by the modified PROMIS subscales and iscorEB.

In some embodiments, rCol7 treatment reduces or stabilizes systemic features of RDEB including dysphagia (e.g., as measured by Brief Esophageal Dysphagia Scale and/or oral nutritional intake), corneal symptoms (e.g., as measured by Eye Symptoms Scale), nutritional status (e.g., as measured by biomarkers Hgb/Hct, total protein/albumin, and/or Fc/TIBC), inflammation (e.g., as measured by markers for inflammation and/or CRP), fibrosis (e.g., as measured by fibrosis biomarkers), DEB associated activity, and/or mental health and social functioning.

In some embodiments, rCol7 treatment improves the overall quality of life, overall health, and disability of a subject with RDEB. In some embodiments, improvement of quality of life, health, and/or disability is measured by a patient interview and/or self- evaluation. In some embodiments, improvement of quality of life, health, and/or disability is measured by a caregiver interview and/or evaluation.

In some embodiments, rCoL7 treatment decreases pain associated with RDEB, decreases blister and/or wound formation, increases the rate of healing of skin blisters and/or skin wounds, increases the rate of wound closure, increases the percentage of wound healing, decreases time to re-blistering and/or rewound, increases skin integrity (i.e., decrease in likelihood of tearing or blistering due to mechanical or environmental stress), decreases the number of chronic blisters and/or wounds, decreases the wound surface area including the total wound area, decreases the number of concomitant medications required to control RDEB symptoms, decreases the number of blisters and/or wounds which become infected, decreases pruritus, decreases inflammation associated with RDEB, and/or decreases infection incidence. In some embodiments, rCol7 treatment reduces the risk of anemia that is typically associated with larger recurrent wound size and longer time to close the open wounds in subjects with RDEB.

### Administration

The methods provided herein of, e.g., treating a skin disorder such as DEB (e.g., RDEB or DDEB) and ameliorating, alleviating, reducing, improving, delaying onset of, delaying progression of, eliminating, and/or curing a symptom or complication of a skin disorder such as DEB may comprise systemic administration of a Col7 drug substance (e.g., rhCol7 substance). Systemic administration is suitable for delivery rhCol7 protein to the skin of the total body in the subject, as well as other areas of the body that may be impacted by a systemic skin disorder such as DEB (e.g., RDEB or DDEB).

In some embodiments, systemic administration of a Col7 substance (e.g., rhCol7substance) comprises intravenous injection or infusion. As a non-limiting example, the rhCol7 composition may be administered by slow infusion via an intravenous route of administration.

In some embodiments, the rhCol7 drug product is administered by bolus injection, slow push injection, or intravenous infusion. In some embodiments, the administration is by intravenous infusion over a period of time, e.g., less than about 6 hours, less than about 5 hours, less than about 4 hours, less than about 3 hours, less than about 2 hours, less than about 90 minutes, less than about 80 minutes, less than about 70 minutes, less than about 60 minutes, less than about 50 minutes, less than about 45 minutes, less than about 30 minutes, less than about 20 minutes, or less than about 10 minutes. For example, the intravenous infusion may last about 1-10 minutes, about 1-5 minutes, about 3-10 minutes, about 5-10 minutes, or 10-30 minutes. In some embodiments, the intravenous infusion lasts about 30minutes to 4 hours, such as about 30-60 minutes, about 30-90 minutes, about 1-4 hours, about 1-3 hours, or about 2-4 hours.

In some embodiments, the rhCol7 protein therapy is administered at an infusion rate of 0.1 mg/kg/minutes to 5.0 mg/kg/minute, such as about 0.1 mg/kg/minute, 0.2 mg/kg/minute, 0.3 mg/kg/minute, 0.4 mg/kg/minute, 0.5 mg/kg/minute, 0.6 mg/kg/minute, 0.7 mg/kg/minute, 0.8 mg/kg/minute, 0.9 mg/kg/minute, 1.0 mg/kg/minute, 1.1 mg/kg/minute, 1.2 mg/kg/minute, 1.3 mg/kg/minute, 1.4 mg/kg/minute, 1.5 mg/kg/minute, 1.6 mg/kg/minute, 1.7 mg/kg/minute, 1.8 mg/kg/minute, 1.9 mg/kg/minute, 2.0 mg/kg/minute, 2.5 mg/kg/minute, 3.0 mg/kg/minute, 3.5 mg/kg/minute, 4.0 mg/kg/minute, 4.5 mg/kg/minute, or 5.0 mg/kg/minute.

### Dosing Regimen

In some embodiments, more than one dose of a rhCol7 drug product is administered to a subject. In some embodiments, a dosing interval (e.g., time between doses during a dosing regimen) is less than about one day, about one day, about 2 days, about 3 days, about 4 days, about 5 days, about 6 days, about one week, about two weeks, about three weeks, about one month, about two months, about three months, about six months, about one year, or longer. In some embodiments, the subject is administered a defined number of doses of a rhCol7 substance in a defined period of time. For example, the subject may receive 2, 3, 4, 5, 6, 7, 8, 9, 10 or more doses over a defined period of time, such as during a one week, two week, three week, four week, five week, six week, month long, year long, or other period.

In some embodiments, rhCol7 treatment lasts at least about one month, such as at least about three months, four months, five months, six months, one year, two years, three years, or longer. In some embodiments, rhCol7 treatment lasts the lifetime of the subject.

In some embodiments, dosing is weight-based dosing. In some embodiments, an rhCol7 substance is administered at a dose of about 0.01 milligrams of drug substance per kilogram of subject (mg/kg) to 50 mg/kg, such as about 0.05 mg/kg to 50 mg/kg, about 0.1 mg/kg to 50 mg/kg, about 0.5 mg/kg to 50 mg/kg, about 1 mg/kg to 50 mg/kg, about 5 mg/kg to 50 mg/kg, about 0.01 mg/kg to 20 mg/kg, about 0.1 mg/kg to 20 mg/kg, about 0.5 mg/kg to 20 mg/kg, about 1 mg/kg to 20 mg/kg, about 0.01 mg/kg to 10 mg/kg, about 0.1 mg/kg to 10 mg/kg, about 0.5 mg/kg to 10 mg/kg, about 1 mg/kg to 10 mg/kg, about 3 mg/kg to 10 mg/kg, about 0.01 mg/kg to 5 mg/kg, about 0.1 mg/kg to 5 mg/kg, about 0.3 mg/kg to 5 mg/kg, about 0.5 mg/kg to 5 mg/kg, about 1.0 mg/kg to 5.0 mg/kg, about 3.0 mg/kg to 5.0 mg/kg, about 0.01 mg/kg to 3 mg/kg, about 0.1 mg/kg to 3 mg/kg, about 0.3 mg/kg to 3 mg/kg, about 0.5 mg/kg to 3 mg/kg, about 1 mg/kg to 3 mg/kg, or any useful range therein. In some embodiments, an rhCol7 substance is administered at a dose of between about 0.3 mg/kg to about 5 mg/kg. In some embodiments, an rhCol7 substance is administered at a dose of about 0.01 mg/kg, about 0.03 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.5 mg/kg, about 5.0 mg/kg, about 6.0 mg/kg, about 7.0 mg/kg, about 8.0 mg/kg, about 9.0 mg/kg, about 10 mg/kg, about 15 mg/kg, or about 20 mg/kg.

In some embodiments, the rhCol7 protein treatment includes thawing and diluting a frozen or lyophilized rhCol7 substance with a pharmaceutically acceptable carrier such as a sterile saline solution suitable for intravenous injection, thereby forming a drug solution; and withdrawing a volume of drug solution to provide a pre-selected dose, e.g., a dose of about 0.01 mg/kg, about 0.03 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.5 mg/kg, about 5.0 mg/kg, about 6.0 mg/kg, about 7.0 mg/kg, about 8.0 mg/kg, about 9.0 mg/kg, about 10 mg/kg, about 15 mg/kg, or about 20 mg/kg.

In some embodiments, a suitable saline solution contains sodium chloride. In some examples, sodium chloride is present at a concentration ranging from approximately 0-2% (e.g., from approximately 0-1.5% or 0-1.0%). In some embodiments, the sodium chloride is present at a concentration of approximately 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0%. In some embodiments, the saline solution is present at a concentration of more than about 2.0%. In one example, the rhCol7 drug product is diluted in 0.9% saline forming drug solution for administration.

In some embodiments, a single dosing interval is used over the course of therapy with a Col7 substance (e.g., rhCol7 substance). In some embodiments, multiple dosing intervals are used over the course of therapy with a Col7 substance (e.g., rhCol7 substance). In some embodiments, a dosing interval is fixed. In some embodiments, a dosing interval is an individualized interval that is determined for a given subject based on, e.g., pharmacokinetic/toxicokinetic (PK/TK) data or other information about that subject, such as age, weight, height, body mass index, disease severity, comorbidities, race, ethnicity, national origin, genotype, and/or prior response to a Col7 replacement therapy. An individualized dose and dosing interval combination may be the same as those for fixed interval regimens or may differ. In some embodiments, the subject initially receives a fixed dosing interval for the first two or more doses, and then changes to an individualized dosing interval depending on a follow-up measurement after the initial dosing. Alternatively, the regimen may initially be at an individualized dosing interval, and then it may change to a fixed dosing interval.

In some embodiments, a single dose level is used over the course of therapy with a Col7 substance (e.g., rhCol7 substance). In some embodiments, multiple dose levels are used over the course of therapy with a Col7 substance (e.g., rhCol7 substance). In some embodiments, the dosing corresponding to a first dosing interval of a first dosing regimen is different from the dosing corresponding to a second dosing interval of a second dosing regimen. In some embodiments, a first dosing regimen comprises dosing the subject at a first dose level and with a first dosing interval and a second dosing regimen comprises dosing the subject at a second dose level and with a second dosing interval, where the first dose level and second dose level are the same. In some embodiments, a first dosing regimen comprises dosing the subject at a first dose level and with a first dosing interval and a second dosing regimen comprises dosing the subject at a second dose level and with a second dosing interval, where the first dose level and second dose level are different. In some embodiments, the first dosing regimen precedes the second dosing regimen, and the first dose level is lower than the second dose level. In some embodiments, the first dosing regimen precedes the second dosing regimen, and the first dose level is higher than the second dose level. In some embodiments, the first dosing regimen precedes the second dosing regimen, and the first dosing interval is shorter than the second dosing interval. In some embodiments, the first dosing regimen precedes the second dosing regimen, and the first dosing interval is longer than the second dosing interval.

In some embodiments, a method of treating a skin disorder (e.g., DEB, such as RDEB or DDEB), or ameliorating, alleviating, reducing, improving, delaying onset of, delaying progression of, eliminating, and/or curing a symptom or complication of a skin disorder such as DEB, comprises administering a Col7 substance (e.g., rhCol7 substance) according to a first dosing regimen and a second dosing regimen, wherein: i) the first dosing regimen includes administering a first therapeutically effective amount of the substance according to a first dosing interval; and ii) the second dosing regimen includes administering a second therapeutically effective amount of the substance according to a second dosing interval. Administration according to the first dosing regimen may take place prior to administration according to the second dosing regimen. The first dosing regimen may be referred to as a “loading phase” and the second dosing regimen may be referred to as a “maintenance phase.” In some embodiments, the first therapeutically effective amount is greater than the second therapeutically effective amount. In some embodiments, the first therapeutically effective amount is less than the second therapeutically effective amount. In some embodiments, the first therapeutically effective amount is the same as the second therapeutically effective amount. In some embodiments, the first dosing interval is the same as the second dosing interval. In some embodiments, the first dosing interval is longer than the second dosing interval (e.g., the first dosing frequency is lower than the second dosing frequency). In some embodiments, the first dosing interval is shorter than the second dosing interval (e.g., the first dosing frequency is higher than the second dosing frequency). In some embodiments, the first dosing interval is a fixed dosing interval and the second dosing interval is an individualized dosing interval. In some embodiments, the first dosing interval is an individualized dosing interval and the second dosing interval is a fixed dosing interval. In some embodiments, the first dosing interval and the second dosing interval are both fixed dosing intervals. In some embodiments, the second dosing regimen lasts longer than the first dosing regimen. In some embodiments, the first dosing regimen lasts longer than the second dosing regimen.

In some embodiments, the first dosing regimen comprises administering at least one dose of the substance to the subject, such as at least 2, 3, 4, 5, 6, 7, 8, 9, 10, 11, 12, 13, 14, 15, or more doses to the subject. In some embodiments, the first dosing regimen comprises administering the Col7 substance to the subject at least once per week, such as at least 2, 3, 4, 5, 6, or 7 times per week. In some embodiments, the first dosing interval is 1 day, 2 days, 3 days, 4 days, 5 days, 6 days, 7 days (e.g., 1 week), 2 weeks, 3 weeks, or 4 weeks. In some embodiments, the first dosing regimen comprises administering the Col7 substance to the subject once per week. In some embodiments, the first dosing regimen comprises administering the Col7 substance to the subject less than once per week, such as once every two weeks, once every three weeks, or once every four weeks. In some embodiments, the first dosing regimen lasts 1-12 weeks, such as 1, 2, 3, 4, 5, 6, 7, 8, 9, 10, 11, or 12 weeks. In some embodiments, the first dosing regimen comprises administering a dose of between about 0.1-50 mg/kg, such as about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5.0 mg/kg, about 5.2, about 5.4 mg/kg mg/kg, about 5.6 mg/kg, about 5.8 mg/kg, about 6.0 mg/kg, about 6.2 mg/kg, about 6.4 mg/kg, about 6.6 mg/kg, about 6.8 mg/kg, about 7.0 mg/kg, about 7.5 mg/kg, about 8.0 mg/kg, about 8.5, about 9.0 mg/kg mg/kg, about 9.5 mg/kg, about 10.0 mg/kg, about 11.0 mg/kg, about 12.0 mg/kg, about 13.0 mg/kg, about 14.0 mg/kg, about 15.0 mg/kg, about 20.0 mg/kg, about 25.0 mg/kg, about 30.0 mg/kg, about 35.0 mg/kg, about 40.0 mg/kg, about 45.0 mg/kg, or about 50.0 mg/kg of the Col7 substance.

In some embodiments, the second dosing regimen comprises administering at least one dose of the substance to the subject, such as at least 2, 3, 4, 5, 6, 7, 8, 9, 10, 11, 12, 13, 14, 15, 20, 25, 30, 35, 40, 45, 50, 52, 60, 70, 80, 90, 100, or more doses to the subject. In some embodiments, the second dosing regimen lasts at least about 1 month, such as at least about 1, 3, 6, 8, 9, 12, 18, 24, 36, or more months. In some embodiments, the second dosing regimen lasts from its commencement to the end of the subject's lifetime. In some embodiments, the second dosing regimen comprises administering the Col7 substance to the subject about every day, about every 2 days, about every 3 days, about every 4 days, about every 5 days, about every 6 days, about once a week, about every other week, about every three weeks, about once a month, about every two months, about every three months, about every six months, or less frequently. In some embodiments, the second dosing interval is 1 day, 2 days, 3 days, 4 days, 5 days, 6 days, 7 days (e.g., 1 week), 2 weeks, 3 weeks, 4 weeks, 1 month, 2 months, 3 months, 6 months, or longer. In some embodiments, the second dosing regimen comprises administering the Col7 substance to the subject about every other week. In some embodiments, the second dosing regimen comprises administering the Col7 substance to the subject about every four weeks. In some embodiments, the second dosing regimen comprises administering a dose of between about 0.1-50 mg/kg, such as about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5.0 mg/kg, about 5.2 mg/kg, about 5.4 mg/kg, about 5.6 mg/kg, about 5.8 mg/kg, about 6.0 mg/kg, about 6.2 mg/kg, about 6.4 mg/kg, about 6.6 mg/kg, about 6.8 mg/kg, about 7.0 mg/kg, about 7.5 mg/kg, about 8.0 mg/kg, about 8.5 mg/kg, about 9.0 mg/kg, about 9.5 mg/kg, about 10.0 mg/kg, about 11.0 mg/kg, about 12.0 mg/kg, about 13.0 mg/kg, about 14.0 mg/kg, about 15.0 mg/kg, about 20.0 mg/kg, about 25.0 mg/kg, about 30.0 mg/kg, about 35.0 mg/kg, about 40.0 mg/kg, about 45.0 mg/kg, or about 50.0 mg/kg of the Col7 substance.

In some embodiments, the second therapeutically effective amount and/or second dosing interval of the second dosing regimen are determined at least in part based on an assessment of the subject during or after the first dosing regimen. In some embodiments, the method further comprises, prior to the second dosing regimen, assessing the subject to, e.g., assess the effectiveness of the Col7 substance and/or the response of the subject to the Col7 substance. In some embodiments, the method further comprises assessing one or more clinical parameters during the first dosing regimen, during the second dosing regimen, and/or between the first and second dosing regimens.

The methods provided herein may include a first dosing regimen (loading phase) and a second dosing regimen (maintenance phase). The loading dosing phase at the beginning of a treatment may comprise an initial intensive period at least in part because of the profound deficit of endogenous collagen 7 in subjects with DEB (e.g., RDEB). Without wishing to be bound by theory, the intensive loading dosing regimen may provide a sufficient amount of rhCol7 to the skin to increase fibril formation. In some embodiments, the loading dosing regimen comprises administration of one dose of the rCol7 composition twice daily, every day, every other day, every three days, every four days, every five days, every six days, every week, or every other week. In some embodiments, the loading dosing regimen includes two, three, four, five, six or more doses, for example, the loading dosing regimen may include three or more doses, or four or more doses, or five or more doses. In some embodiments, the loading dosing regimen lasts for at least 1 week, 2 weeks, 3 weeks, 4 weeks, 5 weeks, 6 weeks, 1 month, 2 months, 3 months, or longer. In some embodiments, the loading dose is about 3 mg/kg rhCol7.

The maintenance dosing regimen may involve less frequent dosing of the subject than the loading dosing regimen. In some embodiments, the maintenance dosing regimen comprises administration of one dose of the rCol7 composition every day, every other day, every three days, every four days, every five days, every six days, weekly, every other week, every three weeks, every four weeks, every month, every two months, every three months, every four months, every five months, every six months, or every year. The rCol7 drug product may be administered at the same dose or alternatively different doses, in the loading and maintenance phases. As a non-limiting example, the dose at the loading dosing phase is about 3.0 mg/kg and the dose administered at the maintenance dosing phase is about 3.0 mg/kg. The maintenance period may last four months, five months, six months, seven months, eight months, nine months, ten months, eleven months, one year, one and half years, two years, five years, ten years, or longer, such as for the life of the patient.

In some embodiments, a dosing regimen (e.g., the first dosing regimen and/or the second dosing regimen) including dose frequency, dose, and duration time is personalized according to the subject's reaction, side effects, and treatment efficacy. In some embodiments, a dosing regimen (e.g., the first dosing regimen and/or the second dosing regimen) including dose frequency, dose, and duration time is fixed (e.g., not individualized based on details of the subject).

In some embodiments, the treatment regimen comprises administration of the rCol7 composition to the patient with RDEB at least one loading dose of about 3.0 mg/kg daily, twice daily, thrice daily, or weekly, and at least one maintenance dose of about 3.0 mg/kg weekly. In some embodiments, the dosing interval of the loading phase is about one day. In some embodiments, the dosing interval of the loading phase is about one week.

In some embodiments, the treatment regimen comprises administration of the rCol7 composition to the patient with RDEB at least one loading dose of about 3.0 mg/kg daily, twice daily, thrice daily, or weekly, and at least one maintenance dose of about 3.0 mg/kg biweekly. In some embodiments, the dosing interval of the loading phase is about one day. In some embodiments, the dosing interval of the loading phase is about one week.

In some embodiments, the treatment regimen comprises administration of the rCol7 composition to the patient with RDEB at least one loading dose of about 3.0 mg/kg daily, twice daily, thrice daily, or weekly and at least one maintenance dose of about 3.0 mg/kg monthly. In some embodiments, the dosing interval of the loading phase is about one day. In some embodiments, the dosing interval of the loading phase is about one week.

In some embodiments, the treatment regimen comprises administration of the rCol7 composition to the patient with RDEB at least one loading dose of about 3.0 mg/kg daily, twice daily, thrice daily, or weekly and at least one maintenance dose of about 3.0 mg/kg at every two months. In some embodiments, the dosing interval of the loading phase is about one day. In some embodiments, the dosing interval of the loading phase is about one week.

In some embodiments, the treatment regimen comprises administration of the rCol7 composition to the patient with RDEB at least one loading dose of about 3.0 mg/kg daily, twice daily, thrice daily, or weekly and at least one maintenance dose of about 3.0 mg/kg at every three months. In some embodiments, the dosing interval of the loading phase is about one day. In some embodiments, the dosing interval of the loading phase is about one week.

In some embodiments, the methods of the present disclosure comprise intravenously administering to a subject a Col7 substance (e.g., rhCol7 substance) according to a first dosing regimen and a second dosing regimen, where the first dosing regimen comprises (i) administration of a first loading dose of about 0.1-5.0 mg/kg, about 0.3-5.0 mg/kg, about 0.5-5.0 mg/kg, about 1.0-5.0 mg/kg, about 0.1-3.0 mg/kg, about 0.3-3.0 mg/kg, about 0.5-3.0 mg/kg, or about 1.0-3.0 mg/kg, such as about 0.1 mg/kg, about 0.3 mg/kg, about 0.5 mg/kg, about 1.0 mg/kg, about 3.0 mg/kg, or about 5.0 mg/kg, of the Col7 substance on the first day of treatment; (ii) administration of a second loading dose of about 0.1-5.0 mg/kg, about 0.3-5.0 mg/kg, about 0.5-5.0 mg/kg, about 1.0-5.0 mg/kg. 0.1-3.0 mg/kg, about 0.3-3.0 mg/kg, about 0.5-3.0 mg/kg, or about 1.0-3.0 mg/kg, such as about 0.1 mg/kg, about 0.3 mg/kg, about 0.5 mg/kg, about 1.0 mg/kg, about 3.0 mg/kg, or about 5.0 mg/kg, of the Col7 substance about one week after administration of the first loading dose; (iii) administration of a third loading dose of about 0.1-5.0 mg/kg, about 0.3-5.0 mg/kg, about 0.5-5.0 mg/kg, about 1.0-5.0 mg/kg, about 0.1-3.0 mg/kg, about 0.3-3.0 mg/kg, about 0.5-3.0 mg/kg, or about 1.0-3.0 mg/kg, such as about 0.1 mg/kg, about 0.3 mg/kg, about 0.5 mg/kg, about 1.0 mg/kg, about 3.0 mg/kg, or about 5.0 mg/kg, of the Col7 substance on the third week of the treatment; and optionally (iv) administration of a fourth loading dose of about 0.1-5.0 mg/kg. about 0.3-5.0 mg/kg, about 0.5-5.0 mg/kg, about 1.0-5.0 mg/kg, about 0.1-3.0 mg/kg, about 0.3-3.0 mg/kg. about 0.5-3.0 mg/kg, or about 1.0-3.0 mg/kg, such as about 0.1 mg/kg, about 0.3 mg/kg. about 0.5 mg/kg, about 1.0 mg/kg, about 3.0 mg/kg, or about 5.0 mg/kg, of the Col7 substance on the fourth week of the treatment. In some embodiments, at least one of the first loading dose, second loading dose, third loading dose, and optional fourth loading dose differs from the other three. In some embodiments, the first loading dose, second loading dose, third loading dose, and optional fourth loading dose are the same. In some embodiments, the first loading dose is about 3.0 mg/kg. In some embodiments, the second loading dose is about 3.0 mg/kg. In some embodiments, the third loading dose is about 3.0 mg/kg. In some embodiments, the fourth loading dose is about 3.0 mg/kg. In some embodiments, the first loading dose, second loading dose, third loading dose, and optional fourth loading dose are each 3.0 mg/kg. In some embodiments, the second dosing regimen (maintenance phase) is performed after completion of the first dosing regimen (loading phase). In some embodiments, the second dosing regimen is performed immediately after completion of the first dosing regimen (e.g., after administration of the last dose of the first dosing regimen and corresponding dosing interval). In some embodiments, a dose holiday is used between the first dosing regimen and the second dosing regimen. In some embodiments, the second dosing regimen comprises administration of a dose of the Col7 substance daily, twice daily, weekly, every other week, every three weeks, every four weeks, every month, every two months, every three months, every four months, every five months, every six months, or every year. In some embodiments, the second dosing regimen comprises administration of about 0.1-5.0 mg/kg, about 0.3-5.0 mg/kg, about 0.5-5.0 mg/kg, about 1.0-5.0 mg/kg, about 0.1-3.0 mg/kg, about 0.3-3.0 mg/kg, about 0.5-3.0 mg/kg, or about 1.0-3.0 mg/kg, such as about 0.1 mg/kg, about 0.3 mg/kg, about 0.5 mg/kg, about 1.0 mg/kg, about 3.0 mg/kg, or about 5.0 mg/kg, of the Col7 substance. In some embodiments, the second dosing regimen comprises administration of about 3.0 mg/kg of the Col7 substance. In some embodiments, the duration of the second dosing regimen is such that the subject receives the maintenance dose for more than one year, or more than five years, or more than ten years, or for the life of the subject.

In some embodiments, a method of treating a skin disorder (e.g., DEB, such as RDEB or DDEB), or ameliorating, alleviating, reducing, improving, delaying onset of, delaying progression of, eliminating, and/or curing a symptom or complication of a skin disorder such as DEB, in a subject (e.g., human subject) in need thereof comprises administering a Col7 substance (e.g., rhCol7 substance) according to a first dosing regimen and a second dosing regimen, wherein: i) the first dosing regimen includes administering a first therapeutically effective amount of the substance according to a first dosing interval; and ii) the second dosing regimen includes administering a second therapeutically effective amount of the substance according to a second dosing interval, wherein: a) the first dosing regimen lasts at least one week, the first frequency is weekly, and the first effective amount is about 0.1-10 mg/kg; and b) the second dosing regimen lasts at least four weeks, the second frequency is every other week, and the second effective amount is about 0.1-10 mg/kg. In some embodiments, administration according to the first dosing regimen is performed prior to administration according to the second dosing regimen. In some embodiments, administration according to the first dosing regimen is performed immediately prior to administration according to the second dosing regimen (e.g., as described herein). In some embodiments, there is no gap between the first dosing regimen and the second dosing regimen (e.g., the second dosing regimen commences after the last dose of the first dosing regimen has been administered and corresponding dosing interval elapsed). In some embodiments, there is a gap of at least one day, such as at least two days, three days, four days, five days, six days, one week, two weeks, or longer, between the first dosing regimen and the second dosing regimen. In some embodiments, the first dosing regimen lasts four weeks. In some embodiments, the second dosing regimen lasts at least seven weeks. In some embodiments, the second dosing regimen comprises administering the Col7 substance to the subject over the lifetime of the subject. In some embodiments, the first effective amount and the second effective amount are the same. In some embodiments, the first effective amount is about 0.1-5.0 mg/kg. about 0.3-5.0 mg/kg, about 0.5-5.0 mg/kg, about 1.0-5.0 mg/kg, about 0.1-3.0 mg/kg, about 0.3-3.0 mg/kg, about 0.5-3.0 mg/kg, or about 1.0-3.0 mg/kg, such as about 0.1 mg/kg, about 0.3 mg/kg, about 0.5 mg/kg, about 1.0 mg/kg, about 3.0 mg/kg, or about 5.0 mg/kg. In some embodiments, the first effective amount is 0.3 mg/kg or 3.0 mg/kg. In some embodiments, the second effective amount is about 0.1-5.0 mg/kg, about 0.3-5.0mg/kg, about 0.5-5.0 mg/kg, about 1.0-5.0 mg/kg, about 0.1-3.0 mg/kg, about 0.3-3.0 mg/kg, about 0.5-3.0 mg/kg, or about 1.0-3.0 mg/kg, such as about 0.1 mg/kg, about 0.3 mg/kg, about 0.5 mg/kg. about 1.0 mg/kg, about 3.0 mg/kg, or about 5.0 mg/kg. In some embodiments, the second effective amount is 0.3 mg/kg or 3.0 mg/kg. In some embodiments, the first effective amount and the second effective amount are the same. In some embodiments, the first effective amount and the second effective amount are both 3.0 mg/kg. In some embodiments, the subject has RDEB. In some embodiments, one or more parameters of the second dosing regimen (e.g., duration, second frequency, second effective amount) is determined at least in part based on assessment of one or more clinical parameters of the subject during or after completion of the first dosing regimen.

### Assessment of Treatment Efficacy

As discussed herein, a treatment regimen may be personalized as to the dose frequency, dosage, and duration time according to the subject's response, side effect, and treatment efficacy. In some embodiments, the subject being treated is evaluated and assessed for treatment efficacy (e.g., between a first dosing regimen and a second dosing regimen); such assessments may be used to individualize the treatment schedule.

DEB patients may have different degrees of disease severity by skin involvement (e.g., mild, moderate, or severe) based on the percentage of body surface area (BSA) involvement represented by active and chronic wounds and supported by, e.g., the classification system described by Fine et al. (J Am Acad Dermatol, 2008, 58(6): 931-502), the contents of which are incorporated by reference herein in their entirety. The changes from a patient's baseline (pre-treatment measurement) may be assessed during and/or following the rhCol7 protein therapy. In some examples, one or more target wounds are selected for treatment efficacy assessment. A wound targeted for assessment may be about 3-100 cm² in size. The wounds may be evaluated with images obtained using medical photography and/or evaluation by a physician. Each wound may be assessed individually one or more times, such as at each dose after the initial dose of the Col7 substance. An assessment of a wound may use a 7-point scale for changes from Baseline (Table 1).

TABLE 1
Wound specific 7-point scale

| Score | Definition |
|---|---|
| 1 | Very much improved |
| 2 | Much improved |
| 3 | Minimally improved |
| 4 | No change |
| 5 | Minimally worse |
| 6 | Much worse |
| 7 | Very much worse |

Skin wounds may also be evaluated using imaging methodologies for quantitative wound surface area (WSA), including whole-body and wound-based medical imaging. In some embodiments, the WSA may be measured as the primary parameter of the treatment. As used herein, WSA is defined as the percentage of the wound affected body surface area (BSA), a calculated measure of WSA divided by estimated BSA. WSA is defined relative to the patient population, by wound boundaries, and other aspects of severity of wound. The wounds may include acute wounds such as blisters, erosions, and crusted erosions, as well as chronic wounds. The WSA methods may be based on the rule of nines body regions by Wallace (The Lancet, 1951), the contents of which are incorporated herein by reference in their entirety, and/or based on the Psoriasis Area and Severity Index (PASI) (Berth-Jones et al., 2006; Langley, 2004). In some examples, the WSA may be measured by wound-based quantitative medical imaging with a handheld 3-dimentional camera.

In addition, a Global Assessment (GA) may be used as the basis for clinical assessment of the average overall severity of the wounds (Table 2).

TABLE 2
Global Assessment (GA)

| Score | Definition |
|---|---|
| 0 = clear | No blisters, no erosions, minimal erythema and/or pigmentary |
| | changes may be present |
| 1 = near clear | 2 or fewer small blisters, faint signs of erosion may be present, barely |
| | perceptible evidence of crusting, slight erythema |
| 2 = mild | predominantly small and some medium blisters, minimal erosions, |
| | clear crusting, definite well-defined erythema |
| 3 = moderate | Mix of small and medium blisters, definite erosions, limited areas of |
| | crusting, marked erythema |
| 4 = severe | Mix of medium and large blisters, marked erosions, ulcerations may |
| | be present, marked and extensive crusting, intense erythema |

During the course of a rCol7 replacement treatment (e.g., as described herein), a biopsy (e.g., punch skin biopsy) may be obtained at intervals, e.g., between doses at during a first dosing regimen (e.g., loading dosing phase) and/or second dosing regimen (e.g., maintenance dosing phase), for directly measuring deposition of Col7 protein into the DEJ of the skin. The measurement can be done with immunofluorescence (DIF) staining using anti-Col7 antibodies. Immunoelectron microscopy (IEM) may also be performed to assess whether functional anchoring fibrils form in the tissue, and if the dermal-epidermal separation is reversed after the rhCol7 protein therapy.

In some embodiments, suction blister time (SBT) can be determined to measure the epidermal-dermal skin adherence, thereby assessing treatment efficacy and for modifying dosing regimen. As the RDEB skin blisters more easily than the healthy skin.

In some embodiments, other complications and symptoms relevant to DEB patient populations are assessed, such as gastrointestinal related clinical manifestations, and other non-skin systemic manifestations, dysphagia (difficulty in swallowing), eye erosion, nail dystrophy, alopecia, and mitten deformities of the hands and feet and skin cancer (see, e.g., Fine et al., J Am Acad Dermatol. 2014, 70:1103-1126). In other embodiments, a global assessment for dermatologic conditions that involve multiple areas of skin involvement such as psoriasis or atopic dermatitis is performed.

In some embodiments, the subject undergoing rCol7 replacement therapy is assessed for overall activity of life using the Epidermolysis Bullosa Disease Activity and Scarring Index (EBDASI) activity subscales, an instrument used to characterize disease severity and response. The subject, before the treatment, may be assessed, and given a baseline status for the overall severity of the patient's EB (e.g., a Global Impression of Severity, GIS) considering the cutaneous and systemic manifestations of DEB. A GIS may be, for example, not assessed, mild, moderate, or severe. After the treatment starts, the subject may be assessed after each dosing, and given an overall status change considering the cutaneous and systemic manifestations of DEB. The status change is referred to as Global Impression of Change (GIC). The change score is from 1 to 7 as indicated in Table 1.

### Combined Treatment

In one aspect, the Col7 substance (e.g., rhCol7 substance) may be used in combination with other one or more other treatments for DEB, such as one or more treatments for skin wounds associated with DEB such as RDEB in a subject.

Other treatments that may be used in combination with the systemic rhCol7 replacement therapy provided herein may include, but are not limited to, topical therapies (e.g., topical cream comprising Col7 as described in U.S. patent application publication No. 2015/313967 or topical therapy comprising Coenzyme Q10 as described in US patent application publication No. 2018/369164, the contents of each of which are incorporated herein by reference in their entirety); cell-based delivery of Col7 (e.g., skin keratinocytes mediated Col7 delivery in the PCT publication No. WO2017/120147, the contents of which are incorporated herein by reference in their entirety); viral vector mediated delivery of Col7 (e.g., herpes simplex virus (HSV) mediated delivery of Col7 in the PCT publication No. WO2017/176336, the contents of which are incorporated herein by reference in their entirety); aminoglycoside agents that induce read-through of COL7A1 premature termination codon (PTC) mutations (e.g., gentamicin and amlexanox, as described in, e.g., Atanasova et al., J Invest Dermatol. 2017, 137(9): 1842-1849); RNA editing therapy (e.g., Bremer et al., Mol Ther Nucleic Acids. 2016; 5:0379); and/or physical interventions including bandages, wraps, sutures, stitches, braces, patches, sealing films and glues (e.g., bandages initially applied as liquids), staples, clips, and the like.

In some embodiments, the systemic rhCol7 protein therapy is used in combination of an anti-infection agent (e.g., an antibiotic), an antioxidant (e.g., Vitamin B3, Vitamin A, or Vitamin C), a growth factor (e.g., FGFP), anti-inflammatory agents, pain relievers, and/or another skin healing agent, such as an antibiotic agent, antiseptic, hydrogel, hydrocolloid, alginate, petroleum jelly, aloe vera, cleansing agent, irrigant, moisturizer, or physical intervention (e.g., as described herein).

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### EXAMPLES

### Example 1: In Vitro Pharmacodynamic Studies

### Platelet Aggregation

The platelet aggregation activity and hemolytic activity might be caused by the homology of the N-terminal non-collagenous domain 1 (NC-1) of C7 alpha-chains to von Willebrand Factor (vWF) (Leineweber S, et al., FEBS Lett. 2011; 585(12): 1748-52).

In this study, the rhCol7 substance was tested for the potential of platelet aggregation in vitro in monkey and human blood samples up to a concentration of 200 micrograms per milliliter (μg/mL). Citrated whole blood samples were collected from 6 human subjects and 6 Cynomolgus monkeys. Platelet rich plasma (PRP) samples were prepared and standardized to reach a platelet concentration between 244×10³ and 267×10³ cells per microliter (cells/μL) in human PRP and between 210×10³ and 255×10³ cells/μL in monkey PRP. Samples were loaded in the aggregometer in duplicate in pre-heated cuvette at 37° C. for 2 minutes, before spiking with 0.9% NaCl (negative control); ReoPro® (inhibition control); collagen (positive control); reference item (buffer); the rhCol7 composition at 2, 50, 100 and 200 μg/mL final concentration in human PRP; or the rhCol7 composition at 2, 25, 62 and 126 μg/mL final concentration in monkey PRP. Immediately after being spiked, the platelet aggregation was monitored for 6 minutes. The results indicate that no activation effect was observed following in vitro treatment of human PRP with 2, 50, 100 and 200 μg/mL rhCol7. rhCol7 also displayed no biologically significant activation effect following in vitro treatment of monkey PRP with 2, 25, 62 and 126 μg/mL rhCol7. Although a slight dose-dependent increase was observed in monkey PRP, this slight increase was not biologically significant as compared to the collagen positive control.

There is no biologically significant increase in platelet activation and aggregation observed in monkey platelet rich plasma (PRP) as compared to the collagen positive control. No platelet activation effect was observed following in vitro treatment of human PRP with rhCol7 substance up to 200 μg/mL.

### Hemolysis

The hemolytic potential of rhCol7 compositions was investigated in vitro in blood from rat, cynomolgus monkey, and human. rhCol7 substance formulated in the vehicle (10 mM sodium phosphate, 5 mM sodium citrate, 70 mM NaCl, 100 mM L-arginine, 1.7% sucrose, and 0.05% (v/v) polysorbate 20 at pH 7.2) was tested in rat whole blood samples from Crl:CD (SD) rats at concentrations of 40, 95, and 185 μg/mL; in non-human primate (cynomolgus monkey) whole blood at concentrations of 25, 62.5, and 125 μg/mL; and in human whole blood at concentrations of 50, 100, and 200 μg/mL. Three concurrent control tests were performed using the control vehicle; positive (deionized water) and negative (0.9% sodium chloride for injection) control articles in each type of blood were tested. The blood and treatment volumes per sample were each 0.5 mL (1.0 mL total) for all tests and were conducted using 5 replicates for each test. Each incubation was carried out at approximately 37° C. for approximately 60 minutes. The percentage hemolysis for each sample was calculated according to the formula modified from Makroo et al. (Asian Journal of Transfusion Science 2011, 5, 15-17).

In rat, non-human primate, and human blood, hemolysis was detected in the positive control group, with mean percent hemolysis values ranging from 34.6% to 37.0%. At all exposure levels in each of the 3 blood matrices, no hemolysis was detected. Mean percent hemolysis values for the treated groups ranged from 0.3% to 0.5%. No hemolysis was detected in the negative or vehicle control groups. Mean percent hemolysis values ranged from 0.3% to 1.2% for the negative control and from 0.3% to 0.6% for the vehicle control. Based on these results, no hemolysis was detected at concentrations up to rhCol7 185 μg/mL in rat blood, concentrations up to rhCol7 125 μg/mL in non-human primate blood, and concentrations up to rhCol7 200 μg/mL human blood.

### Example 2: Preclinical Efficacy Studies of rhCol7 Compositions in a Mouse DEB Model (In Vivo Primary Pharmacodynamic Studies)

The mouse model of DEB (Col7a1^{-/-}) with a targeted inactivation of the COL7A1 gene (Heinonen et al., J Cell Sci. 1999, 112:3641-3648) have no detectable Col7 at the dermal-epidermal BMZ, completely lack anchoring fibrils, and are born with extensive cutaneous blisters on their ventral surface and hemorrhagic blisters on their paws and neck, with mortality typically observed within the first week of life. This DEB mouse model (Col7a1^{-/-}) results in severe blistering that mimics the clinical, histological, and ultrastructural presentation of severe human recessive DEB (RDEB). Using this mouse model, nonclinical efficacy studies were performed for dose-dependent response and efficacy after intravenous administration of the rhCol7 drug product, for studying formation of anchoring fibrils, distribution and localization of rhCol7, and histological and survival effects after rhCol7 intravenous dosing. The study design is summarized in Table 3. In Table 3, doses of 16 μg, 28 μg and 40 μg equate to approximately 8, 14 and 20 mg/kg, respectively, based on 2 g body weight.

TABLE 3
Experimental design of primary pharmacodynamic studies in DEB mice

| | | Method of | Dose and | |
|---|---|---|---|---|
| | Experimental design | administration | concentration | Measurements |
| Study 1 | Single dose on post- | i.v. bolus | Vehicle control PBS | Distribution, |
| | neonatal day 2/3 (PND | (superficial | (phosphate buffered | histological and |
| | 2/3) DEB mice | temporal vein) | saline, 0.5 mM | survival |
| | n = 27 (rhCol7) | | EDTA), 40 μL, | endpoints |
| | n = 20 (FB-Col7 | | rhCol7 (Batch 1) | |
| | n = 13 (control vehicle) | | (16 μg; 40 μL), | |
| | | | FB-rCol7 (16 μg; | |
| | | | 40 μL) | |
| Study 2 | Daily dose for 7 days on | i.d. injection | Control PBS (40 μL), | Formation of |
| | PND 2/3 DEB mice | (dorsum) | rhCol7 (Batch 1) | anchoring fibrils |
| | n = 3 (control vehicle) | | (16 μg; 40 μL), | |
| | n = 5 (FB-Col7) | | FB-rCo17 (16 μg; | |
| | n = 6 (rhCol7) | | 40 μL) | |
| Study 3 | Single dose on PND 2/3 | i.v. bolus | Control PBS (40 μL), | Formation of |
| | DEB mice | (superficial | rhCol7 (Batch 1) | anchoring fibrils |
| | n = 3 (control vehicle) | temporal vein) | (28 μg; 70 μL), | |
| | n = 3 (FB-Col7) | | rhCol7 (Batch 1) | |
| | n = 7 (rhCol7; 40 μg) | | (40 μg; 100 μL) | |
| | n = 3 (rhCol7; 28 μg) | | FB-rCol7 (40 μg; | |
| | | | 100 μL) | |
| Study 4 | Single dose on PND 2/3 | i.v. bolus | Vehicle Control PBS | Distribution, |
| | DEB mice | (superficial | (40 μL); | histological |
| | | temporal vein) | Vehicle 2 (137 mM | improvement |
| | | | sodium chloride, | |
| | | | 2.7 mM potassium | |
| | | | chloride, 10 mM | |
| | | | phosphate, 0.5 mM | |
| | | | EDTA, pH 7.1) | |
| | | | (40 μL); | |
| | | | rhCol7: 2.5 mg/kg | |
| | | | (5 μg/40 μL); or | |
| | | | 8 mg/kg | |
| | | | (16 μg/40 μL), | |
| | | | or 14 mg/kg | |
| | | | (28 μg/70 μL) | |
| | | | (Batch 1) | |
| Study 5 | Repeat-dosing on Days | i.v. bolus | Vehicle Control PBS | Distribution, |
| | 1, 3 and 5 on PND 2/3 | (superficial | (40 μL); | histological |
| | DEB mice | temporal vein) | Vehicle 2 (40 μL); | improvement |
| | n = 3 (control) | | rhCol7: 0.5 mg/kg | |
| | n = 5 (rhCol7, 0.5 mg/kg) | | (40 μL); or 1 mg/kg | |
| | n = 6 (rhCol7, 1 mg/kg) | | (40 μL); or 5 mg/kg | |
| | n = 6 (rhCol7, 5 mg/kg) | | (70 μL) (Batch 2) | |
| Study 6 | Single dose on PND 2/3 | i.v. bolus | Vehicle Control PBS | Distribution, |
| | DEB mice | (superficial | (40 μL); | histological and |
| | n = 11 (control) | temporal vein) | rhCol7 (16 μg; 40 μL) | survival |
| | n = 19 (rhCol7, 16 μg, | | (Batches 1 and 2) | endpoints |
| | 40 μL) (Batch 2) | | | |
| | n = 14 (rhCol7, 16 μg, | | | |
| | 40 μL) (Batch 1) | | | |
| Study 7 | Single dose on PND 2/3 | i.v. bolus | Vehicle (10 mM | Distribution, |
| | DEB mice | (superficial | sodium phosphate, | histological and |
| | n = 10 (control) | temporal vein) | 5 mM sodium citrate, | survival |
| | n = 26 (rhCol7_Batch 3, | | 70 mM sodium | endpoints |
| | 16 μg, 40 μL) | | chloride, 100 | |
| | n = 26 (rhCol7_Batch 4, | | arginine, 50 mM | |
| | 16 μg, 40 μL) | | sucrose, 0.05% | |
| | | | polysorbate 20, pH | |
| | | | 7.1) (40 μL) | |
| | | | rhCol7 (Batches 3 | |
| | | | and 4) (16 μg; 40 μL) | |

In Studies 1-3, neonatal DEB Col7a1^{-/-} mice were administered a single i.v. bolus of phosphate buffered saline (PBS) and formulation vehicle control, rhCol7 compositions (Batches 1, 2, and 3) or reference Col7 (wild type Col7 derived from Fibroblasts; FB-Col7) via the superficial temporal vein. The formation of anchoring fibrils was evaluated after a single dose administration of rhCol7 (i.v.; 16 μg (8 mg/kg)) (Study 3) and after once daily intradermal (i.d.) dosing for 7 days (16 μg (8 mg/kg)) (Study 2).

Distribution of rhCol7 and histological and survival endpoints were evaluated after single dose administration of rhCol7 (28 μg (14 mg/kg), 40 μg (20 mg/kg)) (Studies 1, 6 and 7). Indirect immunofluorescence (IF) staining of tongue tissues obtained from DEB Col7a1-/- mice following i.v. administration with rhCol7 composition, FB-rCol7 or control PBS.

The results demonstrated that the rhCol7 composition was detected at the dermal-epidermal BMZ in tongue and skin in 3 out of 5 rhCol7 composition-dosed and 2 out of 6 FB-rCol7-dosed mice surviving more than 90 days. Both rhCol7 composition and FB-rCol7 distributed to the proper location within the dermal-epidermal junction. The rhCol7 composition was also detected in the esophagus of 2 mice surviving to 18 and 90 days. The results suggest that both rhCol7 and reference collagen 7 (FB-rCol7) distributed to the dermal-epidermal BMZ in tongue and skin following a single i.v. dose (16 μg (8 mg/kg)), while no detectable rhCol7 was found in vehicle control DEB mice.

Transmission electron microscopy of the tongue revealed formation of anchoring fibrils directly beneath the lamina densa of the dermal-epidermal BMZ following a single i.v. dose (28 or 40 μg) of rhCol7 composition and FB-rCol7, while no anchoring fibrils were present in vehicle control mice. The rhCol7 composition administered i.d. (16 μg) for 7 days into dorsum skin, demonstrated formation of cross-banded anchoring fibrils protruding perpendicularly from the dermal-epidermal BMZ (Study 2).

Single dose intravenous administration of rhCol7 composition or FB-rCol7 to Col7a1-/- DEB mice corrected the separation between the epidermis and dermis in tongue tissue. In contrast, PBS injected mice showed pronounced separation of the epidermis from the dermis. The proportion of mice with complete closure between the epidermis and dermis in tongue was 48% (12/25), 67% (12/18), and 0% (0/12) for rhCol7 composition, FB-rCol7, and vehicle control, respectively. The proportion of complete closure was statistically different in rhCol7 composition and FB-rCol7 treated mice compared to vehicle control mice.

Studies have demonstrated that i.v. administration of rhCol7 will restore functional Col7 to the dermal-epidermal BMZ. This is expected to promote the assembly of normal anchoring fibrils, providing stability to dermal-epidermal adhesion at the lamina densa/upper papillary dermis interface, and correct blistering abnormalities and complications in patients with DEB. rhCol7 formed anchoring fibrils, and corrected separation of the epidermis from the dermis in DEB mice.

A single i.v. dose (16 μg (8 mg/kg)) of rhCol7 composition or FB-rCol7 resulted in a statistically significant improvement in survival compared to vehicle controls. The median (interquartilerange) survival time was 4, 8, and 16.5 days for Col7a1-/- mice administered vehicle control, rhCol7 composition, and FB-rCol7, respectively. The proportion of mice surviving beyond 30 days was 0% (0/13), 19% (5/27), and 35% (7/20) respectively for mice administered vehicle control, rhCol7 composition, and FB-rCol7, respectively.

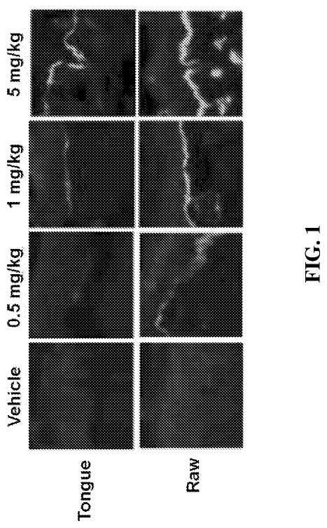

A separate single dose response (5, 16 or 28 μg; equivalent to approximately 2.5, 8 and 14 mg/kg, respectively) and repeat dose response (dosing on Days 1, 3 and 5 at 0.5, 1 and 5 mg/kg) study in the same mouse model of DEB was conducted (Studies 4 and 5) (Table 3). Intravenous administration of the rhCol7 composition resulted in a dose-dependent deposition at the dermal-epidermal BMZ in target tissues following a single dose of 2.5, 8 and 14 mg/kg (tissues collected at death) and repeat-dose administration at 0.5, 1 and 5 mg/kg on Days 1, 3 and 5 (tissues collected on Day 6) (FIG. 1).

Semi-quantification of the IF signal from the repeat-administration dose-response study also showed that rhCol7 distribution to the dermal-epidermal junction was dose dependent;
the minimum efficacious dose was considered to be 5 mg/kg based on distribution to the dermal epidermal BMZ in both tongue and skin. A single dose of the rhCol7 composition resulted in a dose dependent correction of dermal-epidermal separation in tongue.

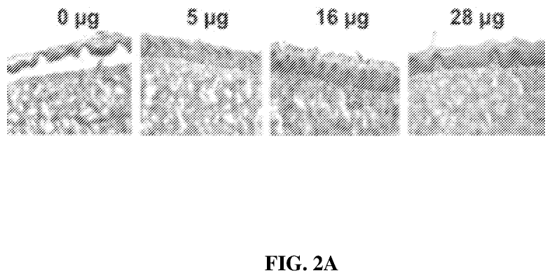

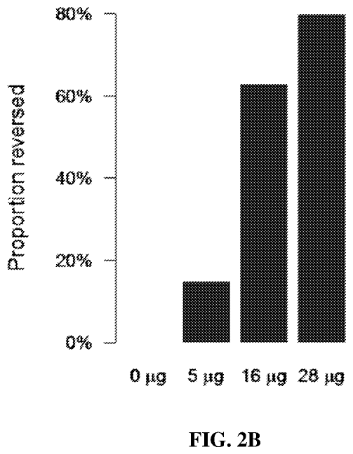

A dose dependent correction of dermal-epidermal separation in neonatal Col7a1-/- mice was also observed following a single intravenous administration of rhCol7 composition (as demonstrated in FIG. 2A). Quantification of the number of mice exhibiting complete closure of dermal-epidermal separation is shown in FIG. 2B.

In studies 6 and 7 for efficacy of rhCol7 compositions, similar distribution results were observed. Following a single dose (8 mg/kg (16 μg)) by intravenous administration, rhCol7 was distributed to the dermal-epidermal BMZ in the tongue in all rhCol7 treated mice, as well as to the skin obtained from the abdomen, back, and front leg in evaluated roCol7 treated mice. rhCol7 was not detected in any vehicle control mice.

Administration of rhCol7 compositions (Batch 1 and 2) resulted in correction of dermal-epidermal separation in Col7a1-/- mice as well. The proportion of mice with complete closure between the epidermis and dermis in tongue was 79% (15/19), 71% (10/14), and 27% (3/11) for mice administered rhCol7 composition (Batch 1), rhCol7 composition (Batch 2) and vehicle control, respectively in Study 6. The proportion of mice with reversal of dermal-epidermal separation was significantly higher in rhCol7 compositions treatment groups compared to vehicle control. In contrast, the proportion of mice with reversal of dermal-epidermal separation was similar between both treatment groups. A statistically significant improvement in survival in rhCol7 dosed groups was observed compared to vehicle controls. The median (interquartile-range) survival time was 3, 4.5, and 11 days for mice administered vehicle control, rhCol7 (Batch 1) and rhCol7 (Batch 2), respectively.

Similar results are observed in study 7. For example, rhCol7 compositions (both batches) distributed to the dermal-epidermal BMZ in tongue and front leg skin, while rhCol7 was not detected in ant vehicle control mice. Correction of dermal-epidermal separation was observed in rhCol7 compositions dosed animals. The proportion of mice with complete closure of dermal-epidermal separation was higher in rhCol7 composition treated mice (46% ( 12/26) (Batch 3), and 58% ( 15/26) (Batch 4)) than vehicle control (10% ( 1/10). A statistically significant improvement in survival of PTR-01 treated animals compared to vehicle controls was observed. The median survival time was 2.5, 9, and 14 days for Col7a1-/- mice administered vehicle control, rhCol7 (Batch 3) and rhCol7 (Batch 4), respectively. A correlation between histology and survival is observed.

These results and observations suggest that rhCol7 compositions by a single intravenous administration can distribute and localize to the proper location within the dermal-epidermal BMZ, form anchoring fibrils, correct dermal-epidermal separation, and result in a statistically significant improvement in survival compared to vehicle controls. Results are similar for mice administered recombinant human rhCol7 purified from human dermal fibroblasts (FB-rhC7), the positive control.

The dose-dependent response is observed both in a dose escalation study utilizing single i.v. administration of rhCol7 composition, and in a dose-dependent study with repeat administration of rhCol7 compositions; the responses include dose-dependent distribution of rhCol7 compositions to the dermal-epidermal BMZ in target tissues and a dose-dependent reversal of dermal-epidermal separation. The observation suggests a minimum efficacious dose is 5 mg/kg based on distribution to the dermal-epidermal BMZ in both tongue and skin.

### Example 3: In Vivo Pharmacokinetics and Toxicokinetics of rhCol7 Compositions

Pharmacokinetic (PK)/toxicokinetic (TK) characteristics of rhCol7 have been assessed in mice, rats, and monkeys, including single dose and 28-days weekly repeat dose studies by the intravenous (i.v.) route. The characterization includes PK/TK evaluations in repeat-dose studies in rats and monkeys, tissue distribution using quantitative whole-body autoradiography (QWBA) in rats and ADA analysis in the repeated dose toxicity. Absorption, distribution, and excretion of rhCol7 compositions were tested in rat, monkey, and dog. The studies for these purposes are listed in Table 4.

TABLE 4
Pre-clinical pharmacokinetic studies with rhCol7 compositions

| | | Method of | Dose and | |
|---|---|---|---|---|
| | Experimental system | administration | concentration | Measurements |
| Study 8 | Rat, Sprague-Dawley | single-dose; i.v. | rhCol7 (0.5, 2 | Absorption |
| | (males only) | | and 5 mg/kg) | |
| | (n = 12/rhCol7group) | | | |
| Study 9 | Monkey, Cynomolgus | single-dose; i.v. | rhCol7 (0.5, 2 | Absorption |
| | (females only) | | and 5 mg/kg) | |
| | (n = 3/rhCol7group) | | | |
| Study 10 | Rat, Sprague-Dawley | 5 doses/28 days, | rhCol7 (2, 5 | Absorption |
| | (n = 12 2 mg/kg rhCol7, | i.v. | and 10 mg/kg) | |
| | n = 12, 5 mg/kg rhCol7 | | control | |
| | n = 12, 10 mg/kg rhCol7 | | vehicle | |
| | n = 8 control) | | | |
| Study 11 | Monkey, Cynomolgus | 5 doses/28 days, | rhCol7 (2, 5 | Absorption |
| | (n = 8, 2 mg/kg rhCol7 | i.v. | and 10 mg/kg) | |
| | n = 14, 5 mg/kg rhCol7 | | | |
| | n = 14, 10 mg/kg rhCol7 | | | |
| | n = 14 control) | | | |
| Study 12 | Monkey, Cynomolgus | 5 doses/28 days, | rhCol7 (4 and | Absorption |
| | (n = 12, 4 mg/kg rhCol7 | i.v. | 8 mg/kg) | |
| | n = 12, 8 mg/kg rhCol7) | | | |
| Study 13 | Mouse, Col7a1 −/− | i.v. | | Half-life in tongue |
| | (DEB model) | | | and skin |
| Study 14 | Mouse, tamoxifen- | 2 doses at | | Distribution in |
| | inducible C7 knockout | 20 minutes | | tongue, esophagus |
| | (DEB model) | intervals; i.v. | | and skin (intact and |
| | | | | wound) |
| Study 15 | Rat, Sprague-Dawley | single-dose; i.v. | rhCol7 | Tissue distribution; |
| | (n = 3) | | (2 mg/kg) | and excretion (urine |
| | | | | and feces) |
| Study 16 | Dog, RDEB model | single-dose; i.v. | | Distribution in skin |
| | | | | and lip |

### Single Dose-Absorption in Rats and Monkeys

After single i.v. slow push (1-3 minutes) administration of 0.5, 2 and 5 mg/kg to male Sprague-Dawley Crl:CD rats (12 males/group) (study 8). Blood samples were collected prior to dosing and at approximately 2, 5, 15, 30, and 45 minutes, 1, 2, 4, 8, 12, and 24 hours after dose administration on Day 0. Serum samples were analyzed with electrochemiluminescence (ECL). Exposure to rhCol7 composition increased with the increase in rhCol7 dose from 0.5 to 5 mg/kg in a greater than dose-proportional manner in terms of area under the curve from time zero to infinity (AUC_{inf}) and dose-proportional in terms of Cₘₐₓ. However, the increase in exposure in terms of AUC_{inf} was dose-proportional from 2 to 5 mg/kg. A 10-fold increase in dose resulted in approximately 9- and 33-fold in terms of Cₘₐₓ and AUC_{inf}, respectively. Peak serum concentrations of rhCol7 were observed at the first sampling point (2 minutes) post-dose at all dose levels. Cl was low ranging from 83.7 to 276 mL/h/kg. and the volume of distribution at steady state (Vₛₛ) was small, ranging from 17.1 to 33.1 mL/kg. The resulting terminal half-life (T_{1/2}) was short, ranging from 0.108 to 0.293 hour. Cl appeared to slightly decrease from 0.5 to 2 mg/kg and remained similar at 2 and 5 mg/kg. At 5 mg/kg the maximum serum concentration (Cₘₐₓ) was 105,000 ng/mL and AUC₀₋₂₄ was 59,800 ng*hr/mL.

Similarly, after single i.v. slow push (1-2 minutes) administration of 0.5, 2, and 5 mg/kg rhCol7 at a dose volume of 10 mL/kg, to female Cynomolgus monkeys (3 females/group) (study 9). Blood samples were collected prior to dosing and at approximately 2, 5, 15, 30, and 45 minutes and 1, 2, 4, 8, 12, and 24 hours after dose administration on Day 0. Serum samples were analyzed with ECL. rhCol7 serum concentrations were measurable in at least 1 of 3 animals through 4-, 12-, and 24-hours post-dose at 0.5, 2, and 5 mg/kg, respectively. Peak serum concentrations of rhCol7 were observed at the first sampling point (2 minutes) post-dose at all dose levels. Serum concentrations appeared to decrease in a bi-phasic manner. Exposure to rhCol7 increased with the increase in rhCol7 dose from 0.5 to 5 mg/kg in a greater than dose-proportional manner in terms of AUC_{inf} and dose-proportional in terms of Cₘₐₓ. The increase in exposure in terms of AUC_{inf} was nearly dose-proportional from 2 to 5 mg/kg. Plasma clearance (Cl) was low, ranging from 43.7 to 163 mL/h/kg and Vₛₛ was small, ranging from 32.2 to 51.9 mL/kg. The resulting tin ranged from 0.347 to 5.59 hours. At 5 mg/kg the Cₘₐₓ was 135,000 ng/ml and AUC₀-24 was 116,000 ng*hr/mL.

### Repeat Dose-Absorption in Rats and Monkeys

After repeated once weekly i.v. bolus injections of 2, 5 and 10 mg/kg/week (5 doses in total over a 28-day dosing period) at a dose volume of 10 mL/kg to Sprague-Dawley Crl: CD rats (n=12/dose) (study 10). A concurrent control group (n=8) received the vehicle on the same regimen. Blood samples were collected at approximately 5 and 15 minutes after dose administration on Days 0 and 28 for the control group and prior to dosing and at approximately 5 and 15 minutes and approximately 1, 2, 4, 8, 12, and 24 hours after dose administration on Days 0 and 28. Scrum samples were analyzed with ECL.

rhCol7 serum concentrations were below the level of quantification (BLQ) in all samples prior to dosing with rhCol7 compositions and in samples collected from control animals on Days 0 and 28. In general, exposures were similar on Day 0 and Day 28 for all dose groups and accumulation of rhCol7 compositions was not observed after once weekly administration for 28 days. Accumulation ratios ranged from 0.516 to 1.27. Clearance was low (approximately 1.07 to 3.64 mL/min/kg for combined male and female data) and Vₛₛ was small (approximately 34.9 to 94.4 mL/kg for combined male and female data) which resulted in a mean terminal tv2 range of approximately 0.196 to 0.787 hours for combined male and female data. No gender difference was observed. The TK parameters of rhCol7 are summarized in Table 5.

TABLE 5
Toxicokinetic parameters for rhCol7 compositions after
intravenous Bolus administration in Sprague-Dawley Rats

| | Dose (mg/kg/day) | | | | | |
|---|---|---|---|---|---|---|
| | 2 | | 5 | | 10 | |
| PK Parameters | Male | Female | Male | Female | Male | Female |
| | Day 0 | | | | | |
| AUC₀₋₂₄ (ng*hr/mL) | 13,500 | 13,500 | 50,800 | 36,600 | 126,000 | 123,000 |
| AUC_{inf} (ng*hr/mL) | 13,400 | 13,400 | 50,700 | 36,400 | 126,000 | 123,000 |
| Cₘₐₓ (ng/mL) | 20,400 | 29,400 | 56,700 | 47,300 | 96,500 | 109,000 |
| Cl (mL/h/kg) | 2.48 | 2.49 | 1.64 | 2.29 | 1.32 | 1.36 |
| Vₛₛ (mL/kg) | 0.0407 | 0.0284 | 0.0497 | 0.0596 | 0.0686 | 0.0664 |
| T_{1/2}(hr) | 0.214 | 0.147 | 0.297 | 0.256 | 0.628 | 0.362 |
| | Day 28 | | | | | |
| AUC₀₋₂₄ (ng*hr/mL) | 11,800 | 6,970 | 64,500 | 24,700 | 160,000 | 151,000 |
| AUC_{inf} (ng*hr/mL) | 11,600 | 6,910 | 64,300 | 24,600 | 160,000 | 151,000 |
| Cₘₐₓ (ng/mL) | 14,000 | 21,100 | 63,300 | 47,900 | 136,000 | 98,900 |
| Cl (mL/h/kg) | 2.86 | 4.82 | 1.30 | 3.39 | 1.04 | 1.11 |
| Vₛₛ (mL/kg) | 0.0996 | 0.0492 | 0.0597 | 0.113 | 0.0654 | 0.0669 |
| T_{1/2}(hr) | 0.349 | 0.260 | 0.462 | 0.395 | 0.794 | 0.777 |

Two studies were conducted in monkeys to investigate the toxicokinetic profile of rhCol7 compositions. In study 11, after repeated once weekly i.v. slow push (1-2 minutes) injections of 2, 5 and 10 mg/kg/week (5 doses in total over a 28-day dosing period) at a dose volume of 10 mL/kg to Cynomolgus monkeys. Blood samples were collected once during acclimation, prior to first dosing, prior to dosing on day 28, and at approximately 5 and 15 minutes and 1, 2, 8, 12, and 24 hours after dose administration on days 0 and 28. Serum samples were analyzed with ECL. There were no measurable rhCol7 concentrations in serum samples collected from control group animals. In general, exposure to rhCol7 (AUC and Cₘₐₓ) was similar in males and females and there was no consistent gender difference.

Following i.v. bolus administration of rhCol7 composition at 2 mg/kg/week, rhCol7 serum concentrations were measurable from 0.083 hours post-dose through 2-, 4-, or 8-hours post-dose on both days. In the 5 and 10 mg/kg/week dose groups, rhCol7 serum concentrations were measurable from 0.083 hours post-dose through 4-, 8-, 12-, or 24-hours post-dose on both days.

The accumulation ratios ranged from 0.738 to 1.50. Clearance was low (<5 mL/min/kg) and Vₛₛ was small (<0.105 L/kg) which resulted in a relatively short mean ty, that ranged from 0.304 to 1.36 hours for combined male and female data. The male and female toxicokinetic parameters for rhCol7 are summarized in Table 6.

TABLE 6
Toxicokinetic parameters for rhCol7 compositions after intravenous slow push administration in monkeys

| | Dose (mg/kg/day) | | | | | |
|---|---|---|---|---|---|---|
| | 2 | | 5 | | 10 | |
| PK Parameters | Male | Female | Male | Female | Male | Female |
| | Day 0 | | | | | |
| AUC₀₋₂₄ (ng*hr/mL) | 21,500 | 29,800 | 157,000 | 120,000 | 415,000 | 439,000 |
| AUC_{inf} (ng*hr/mL) | 21,300 | 29,700 | 157,000 | 120,000 | 414,000 | 438,000 |
| Cₘₐₓ (ng/mL) | 47,200 | 55,700 | 120,000 | 124,000 | 166,000 | 256,000 |
| Cl (mL/h/kg) | 0.083 | 0.125 | 0.536 | 0.131 | 0.155 | 0.131 |
| Vₛₛ (mL/kg) | 1.56 | 1.19 | 0.576 | 1.07 | 0.449 | 0.410 |
| T_{1/2}(hr) | 0.0384 | 0.0338 | 0.0370 | 0.139 | 0.0489 | 0.0379 |
| | Day 28 | | | | | |
| AUC₀₋₂₄ (ng*hr/mL) | 16,900 | 20,000 | 182,000 | 133,000 | 449,000 | 446,000 |
| AUC_{inf} (ng*hr/mL) | 16,700 | 19,700 | 181,000 | 132,000 | 449,000 | 446,000 |
| Cₘₐₓ (ng/mL) | 27,800 | 38,400 | 126,000 | 108,000 | 197,000 | 213,000 |
| Cl (mL/h/kg) | 0.125 | 1.125 | 0.762 | 0.262 | 0.178 | 0.702 |
| Vₛₛ (mL/kg) | 2.12 | 6.06 | 0.501 | 0.663 | 0.681 | 0.374 |
| T_{1/2}(hr) | 0.0789 | 0.125 | 0.0308 | 0.0407 | 0.116 | 0.0383 |

In study 12, after repeated once weekly i.v. infusions (1 hour) of 4 and 8mg/kg/week (5 doses in total over a 28-day dosing period) at an infusion rate of 6.6 mL/kg/h to Cynomolgus monkeys. Blood samples were collected prior to dosing, 30 minutes post start of infusion and at 5 and 15 minutes, and 1, 2, 4, 8, 12, and 24 hours post end of infusion. Serum samples were analyzed with ECL. Over the dose range, exposure to rhCol7 (based on AUC_{0-Tlast}, AUC_{inf} and Cₘₐₓ values) on Days 1 and 29 generally increased dose-dependently and in slightly to moderately more than dose-proportional manner. There were generally no marked gender-related differences. The AUCs and Cₘₐₓ accumulation ratios (Day 29/Day 1) ranged from 0.9 to 1.7 indicating that rhCol7 does not accumulate when administered once weekly as i.v. infusion over a period of 1 hr/day to Cynomolgus monkeys for 4 weeks for a total of 5 doses

The mean estimated ti ranged from 0.359 to 0.857 hours on Day 1 and from 0.427 to 1.40 hours on Day 29. rhCol7 was cleared at a mean rate of 43.8 to 71.9 mL/hour/kg on Day 1 and a mean rate of 27.1 to 54.4 mL/hour/kg on Day 29. The mean volume of distribution during terminal phase (V_{z}) ranged from 31.0 to 52.4 mL/kg on Day 1 and from 31.3 to 47.8 mL/kg on Day 29, suggesting that rhCol7 is confined to a large extent in the circulating system. The TK parameters for rhCol7 are summarized in Table 7.

TABLE 7
Toxicokinetic parameters for rhCol7 after intravenous infusion in monkeys

| | Dose (mg/kg/day) | | | |
|---|---|---|---|---|
| | 2 | | 8 | |
| PK Parameters | Male | Female | Male | Female |
| | Day 1 | | | |
| AUC_{0-Tlast} (ng*hr/mL) | 74,600 | 58,400 | 187,000 | 178,000 |
| AUC_{inf} (ng*hr/mL) | 74,800 | 57,900 | 187,000 | 173,000 |
| Cₘₐₓ (ng/ml) | 57,000 | 42,800 | 80,600 | 86,500 |
| Tₘₐₓ (hr) | 1.11 | 1.11 | 1.08 | 1.14 |
| Cl (mL/h/kg) | 55.0 | 71.9 | 43.8 | 47.9 |
| Vz (mL/kg) | 31.0 | 36.8 | 52.4 | 42.1 |
| T_{1/2} (hr) | 0.391 | 0.359 | 0.857 | 0.628 |
| | Day 29 | | | |
| AUC_{0-Tlast} (ng*hr/mL) | 88,000 | 76,700 | 326,000 | 181,000 |
| AUC_{inf} (ng*hr/mL) | 88,700 | 77,000 | 326,000 | 187,000 |
| Cₘₐₓ (ng/ml) | 50,300 | 49,500 | 108,000 | 82,600 |
| Tₘₐₓ (hr) | 1.22 | 1.12 | 1.17 | 1.33 |
| Cl (mL/h/kg) | 48.4 | 54.4 | 27.1 | 44.0 |
| Vz (mL/kg) | 42.3 | 31.3 | 47.8 | 36.8 |
| T_{1/2} (hr) | 0.631 | 0.427 | 1.40 | 0.612 |

### Distribution

The half-life (T_{1/2}) of rhCol7 compositions distributed in tongue and skin was determined in the DEB mouse model (Col7a1-/1) following a single i.v. administration of rhCol7 composition (8 mg/kg (16 μg) (Study 13). Tongue (n=25) and skin from front and rear paws (n=27) were collected and subjected to IF staining for rhCol7. Tissue T_{1/2} was estimated with semi-quantitated IF signal at the dermal-epidermal BMZ specifically. The tissue t1/2 in tongue and skin obtained from paws was estimated to be 26.4 days and 32.6 days respectively. The distribution of rhCol7 in tongue, esophagus and skin (intact and wounded) was further determined in the tamoxifen-inducible Col7 knockout DEB mouse model following i.v. administration (study 14). After a small wound was incised on the back of the C7-inducible knockout mice, two i.v. injections of 100 μg rhCol7 (8 mg/kg (200 μg in total; n=5) or vehicle (n=4) were administered. Two weeks after rhCol7 placebo injection, the mice were sacrificed and samples of tongue, esophagus and skin (intact and wounded) were collected for rhCol7 detection at the dermal-epidermal junction. rhCol7 distributed to the dermal-epidermal BMZ in wounded skin of tamoxifen-inducible Col7 knockout mice following i.v. administration. rhCol7 was not detected in tongue, esophagus or unwounded skin.

The distribution of [¹¹¹In]-PTR-01 after a single i.v. administration to 8 Sprague Dawley male rats at 2 mg/kg was assessed (Study 15). One animal/time point was sacrificed at 0.5-, 2-, 6-, 24-, 72-, 168-, 216-, and 336-hours post-dose. Following administration of [¹¹¹In]-PTR-01 to rats the Cmax in blood and plasma were observed at 0.5 hours post-dose. The radioactivity concentrations in plasma were approximately 2-fold higher than those in blood. Levels of radioactivity in both matrices declined rapidly and were approximately 5-fold lower by 2 hours post-dose but were still detectable at 336 hours post-dose. The highest radioactivity concentrations and percentage of dose/g were observed in the bone marrow, kidneys, liver, and spleen. The majority of the tissues had tissue: plasma concentration ratios >1 at the later time points indicating that drug-related radioactivity was retained in tissues. rhCol7 was detected in skin (unpigmented) at each time point including 336 hours post-dose; the skin: plasma ratio at 336 h post-dose was 4.61.

The distribution of rhCol7 in the lip and skin (car) was determined in one female RDEB dog following i.v. infusion of 4 mg/kg (study 16). The RDEB dog model is a spontaneous model with a homozygous glycine mutation in the collagenous domain (G1906S). rhCol7 was detected faintly at the dermal-epidermal BMZ in skin (car) 3 days post-dose. A very weak IF signal at the dermal-epidermal BMZ in skin was also detected 25 days post-dose. In contrast, IF staining without the primary antibody (immunoglobulin G (IgG) control) showed no staining at the dermal-epidermal BMZ. rhCol7 was detected faintly in the skin car) but not in the lip. There were no changes in anchoring fibrils and the suction blister test in the one animal dosed.

### Excretion

rhCol7 tissue distribution and excretion was studied in [¹¹¹Indium (In)]-rhCol7 treated rat (study 15). Sprague Dawley male rats was administered with [¹¹¹Indium (In)]-rhCol7 b y a single i.v. injection at a dose level of 2 mg/kg. Urine was collected from animals at 0-6 and 6-24 hours post-dose and thereafter at 24-hour intervals through 168 hours post-dose. Feces were collected at 24-hour intervals through 168 hours post-dose. Concentrations of total radioactivity were determined in dose formulations using instant thin layer chromatography (iTLC) and solid scintillation counting (SSC), and quantitative whole-body autoradiography (QWBA). The autoradiographic images were analyzed to determine tissue concentrations of radioactivity.

Blood, plasma, urine, feces, and carcasses were analyzed by SSC to determine radioactivity concentrations. After i.v. administration of [¹¹¹In]-rhCol7 the highest recovery of drug-related radioactivity, accounting for a mean of 56.8% over the 0 to 168 hour collection period, was found in the urine. Drug-related radioactivity was also present in feces. Approximately 30% of the administered dose remained in the carcass and total recoveries (urine, feces, and carcasses) were a mean of 95.9±1.04%.

In summary, there were no general gender differences in systemic exposure as measured by AUC and Cₘₐₓ in either rats or monkeys. Elimination after i.v. administration was rapid, with tin often less than half an hour in all species tested. In both rodents and non-human primates rhCol7 half-life in plasma increased with dose. Exposure data support allometric scaling for human equivalent dose (HED) calculation. In the tamoxifen-inducible Col7 knockout mouse model rhCol7 distributed to the dermal-epidermal BMZ in wounded skin further confirming the specific homing of collagen VII. This is similar to the tissue half-life of endogenous murine Col7 in target tissues (skin, tongue and esophagus), as estimated in a tamoxifen-inducible Col7 knockout mouse model (Kuhl et al., J Invest Dermatol., 2016, 136:1116-1123). rhCol7 was widely distributed throughout the whole body of the rat after an i.v. administration. The highest amount of rhCol7 was observed in the bone marrow, kidneys, liver and spleen. In Sprague Dawley rats, levels of [¹¹¹In]-rhCol7 in blood and plasma declined rapidly and were approximately 5-fold lower by 2 hours post-dose but were still detectable at 336 hours post-dose after i.v. administration. rhCol7 was detected faintly at the dermal-epidermal BMZ in skin 3 days following a single i.v. infusion.

### Example 4: Selection of Patients Prior to the rhCol7 Drug Treatment

Recessive dystrophic epidermolysis bullosa (RDEB) patients with at least one chronic (e.g., at least 6 weeks) lesion >2 cm² are selected. Patients diagnosed with RDEB have a genetic analysis before enrollment for the rhCol7 protein treatment. A blood sample is collected from each patient for genotyping of Col7A1. Only patients showing a mutation(s) in the COL7A1 gene consistent with a recessive inheritance pattern, are enrolled. Biopsy samples are also obtained from patients for IF staining prior to the enrollment. The patients who have the presence of some but deficient Col7 staining at the dermal-epidermal junction (DEJ) by IF staining are selected.

### Example 5: Efficacy of rCol7 Replacement in Human Patients with RDEB

This study was performed to assess the efficacy of rCol7 replacement in treating humans with RDEB. Adult Patients with Recessive Dystrophic Epidermolysis Bullosa (RDEB) are selected as described in Example 4. Twelve patients with a diagnosis of RDEB and a history of at least one chronic lesion were enrolled and divided into 4 dosing cohorts. Patients received 3 doses of PTR-01 at doses ranging from 0.1 to 3.0 mg/kg. In particular, the four cohorts were dosed at 0.1 mg/kg (active drug), 0.3 mg/kg (active drug), 1.0 mg/kg (active drug), and 3.0 mg/kg (active drug), respectively.

Efficacy assessments were performed prior to first dose of therapy, and repeatedly during the treatment, and follow-up periods on Days 15, 29, 43, 57, 71, 85 and 127.

The pharmacokinetic parameter estimates including Cₘₐₓ, Tₘₐₓ, AUC, clearance, and tin were measured. Skin samples were obtained and subject to IF staining for Col7 staining at the dermal-epidermal junction (DEJ). Electron microscopy (EMS) was also used to assess anchoring fibrils in the skin biopsy. The duration of rhCol7 in tissues was measured in the skin biopsy.

Changes of skin blisters, skin lesion size, and wound healing were assessed and measurements of biomedical markers including albumin, iron/TIBC, hemoglobin, and hematocrit were also tested. Additionally, patient reported outcomes such itch and pain scale, overall life quality, and DEB activity, etc., were included as part of the assessment,

The interim efficacy assessment data indicated a 200% increase, on average, in C7 deposition at the dermal-epidermal junction (DEJ) by direct immunofluorescence (DIF) was seen at 1 mg/kg dose. A dose-dependent increase in C7 deposition at the DEJ was shown with the C7 levels at 3 mg/kg approaching those seen in asymptomatic heterozygotes and normal human skin.

A trend towards improved wound healing in patients was observed. A dose-dependent increase in this study with Cₘₐₓ levels at 3 mg/kg approaching those seen at the minimum effective dose in animals. In addition, the increase in C7 DIF was maintained for about one month after discontinuation of dosing.

### Example 6: Dosing Frequency Study in Patients with RDEB

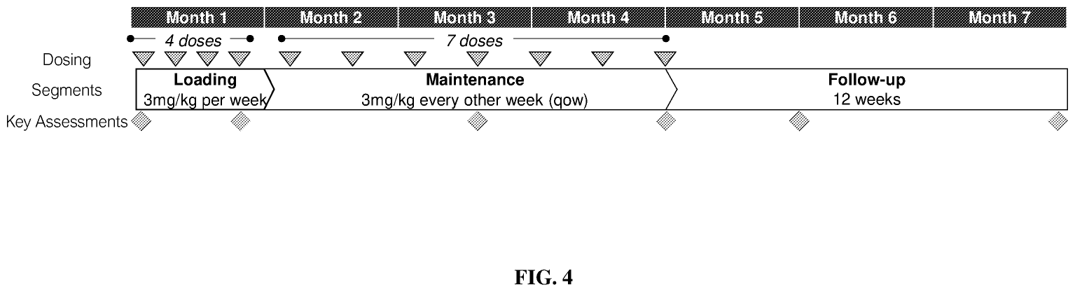

This Phase 2, Open-Label study determines the dose frequency on incorporation of rC7 in the skin and efficacy in patients with RDEB. The treatment plan to RDEB patients were divided into 3 parts including two dosing regimens (Parts 1 and 2, alternately referred to as “loading” and “maintenance” phases) and a 3-month observation phase (Part 3). In Part 1. patients receive a dose of 3.0 mg/kg rCol7 every week for a total of 4 doses. In Part 2. following Part I, patients receive a dose of 3.0 mg/kg rCol7 every other week for a total of 7 doses. An efficacy assessment is performed at the end of each dosing period. During Part 3, patients are evaluated at Months 1 and 3 after completion of dosing to access the durability of wound healing and other efficacy parameters. All the doses were administered by intravenous infusion. FIG. 4 schematically illustrates the study design.

Dosing is scheduled as follows: For patients over 16 years of age and older, PTR-01 was administered at a dose of 3.0 mg/kg IV weekly for 4 doses, then every other week for 7 doses; and for patients under 16 years of age, PTR-01 is administered at a dose of 120 mg/m2 IV weekly for 4 doses, then every other week for 7 doses.

Primary endpoints for this study included improvement in a majority of target lesions of at least 2 levels using a 7-point Global Impression of Change (GIC) instrument, as well as treatment-emergent adverse events (TEAEs), infusion-associated reactions (IARs), and immunogenicity. Secondary and exploratory endpoints included delivery of PTR-01 to skin; formation of new anchoring fibrils as measured by electron microscopy; wound area of target lesions by imaging; investigator GIC (IGIC) assessment; total body wound surface area; severity of pain and impact of pain on quality of life (modified PROMIS subscales and iscorEB); global impressions of severity and change (IGIS&C, PGIS&C); wound care burden; patient interviews and anecdotal reports; and markers of skin fibrosis.

Table 8 summarizes details of patients enrolled in the study. Six patients were enrolled and five completed the study. Patients 202-001, 202-002, and 202-003 participated in the Phase 1 study described in Example 5. The median patient age was 19.0 years.

TABLE 8
Details of study patients

| | 201-001 | 202-001 | 202-002 | 202-003 | 203-001 | 203-002 |
|---|---|---|---|---|---|---|
| Age | 30 | 20 | 17 | 30 | 18 | 13 |
| Gender | Female | Male | Male | Male | Male | Male |
| Ethnicity | White | White | White | Non-White | White | White |
| Mutation | Homozygous | Homozygous | Heterozygous | Heterozygous | Heterozygous | Unknown |
| | p.GLU2020ASN | R578X | c.7787delG | c.4448G > A; | c.993C > G | p.G2013S |
| | FSX183; | | (exon 104); | c.8221C > T | (p.Y311*); | (exon 73); |
| | p.GLU2020ASN | | IVS26-3T -->g | | c.7929 + | n/a |
| | FSX183 | | (intron 26) | | 11_7929 + 26DEL | |

Table 9 summarizes wound response on Day 120 compared to baseline by Wound-Specific 7 Point Scale. In the table, m is the number of wounds that met the response criteria; M is the total number of wounds; responders were patients with a ≥2-point increase in the wound-specific 7-point assessment scale in ≥50% of their wounds; and the total wound response is the total number of wounds (all patients) with a ≥2-point increase in the wound-specific 7-point assessment scale for all patients. The total number of patients assessed was 5rather than the 6 enrolled in the study as one patient discontinued after Day 36 due to lack of efficacy.

TABLE 9
Wound response on Day 120 compared to baseline

| | Overall |
|---|---|
| Parameter | (N = 6) |
| Number of patients with Day 120 assessment | 5* |
| Responders [n (%)] | 4 (66.7) |
| Non-responders [n (%)] | 1 (16.7) |
| Response Rate (95% CI) | 80.0 (28.4, 99.5) |
| Total wound response for all patients [m/M (%)] | 18/26 (69.2) |

*One patient discontinued after day 36 due to lack of efficacy.

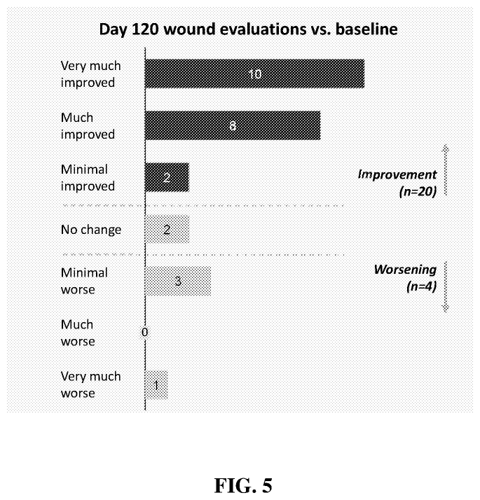

FIG. 5 shows the comparison of Day 120 wound evaluations vs. baseline. As shown in Table 9 and FIG. 5, treatment with PTR-01 led to rapid, consistent, and durable wound healing. By Day 15, 15/26 wounds (57.7%) met the response criteria of ≥2-point increase in the wound-specific 7-point assessment scale, and at Day 120, 18/26 wounds (69.2%) did. Based on these criteria, 4/6 patients (66.7%) were responders.

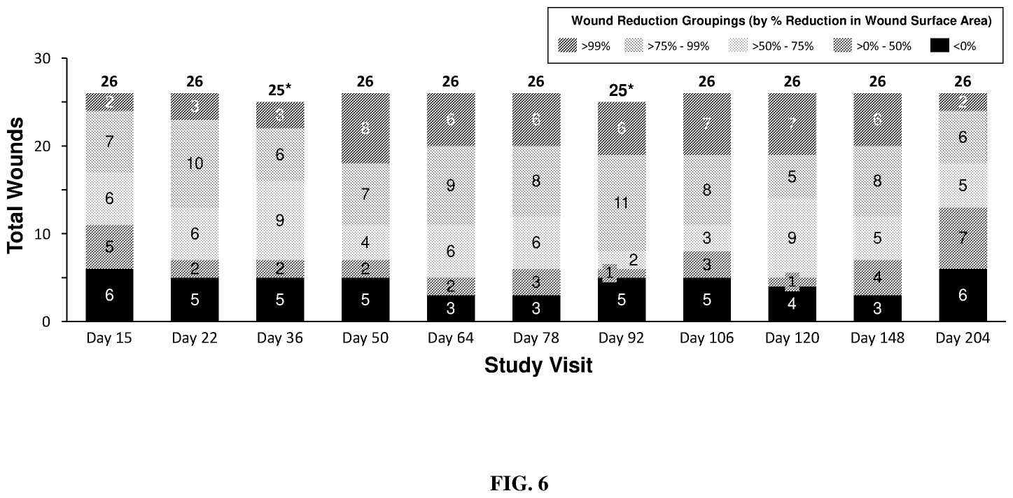

FIG. 6 shows the wound response by percent reduction in wound surface area by Canfield imaging. Wounds exhibited a rapid response to treatment with a majority (80%) reaching >50% closure by Day 68. At Day 120, the end of treatment, over 80% of wounds closed with >50% compared to baseline. Durability of treatment lasted one month after the last dose with treatment effects waning starting at Day 204.

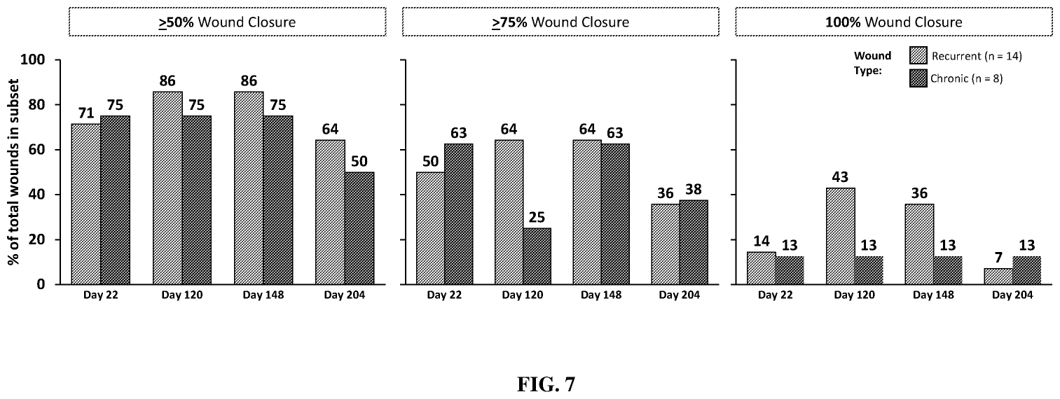

FIG. 7 shows the wound closure observed in chronic and recurrent wounds. PTR-01 impacts wound closure in chronic wounds, which are a major source of morbidity. This analysis was based on 14 wounds characterized as “recurrent” and 8 wounds characterized as “chronic;” efficacy was similarly observed in small and large wounds. Of 26 wounds analyzed in the study, 4 were labeled as “physician choice” per protocol and therefore no clinical wound history was provided and the wounds were omitted from the analysis. Robust wound healing response was observed across different wound types: small and large, chronic and recurrent. The majority of wounds achieved healing of ≥50 and ≥75% on Day 120.

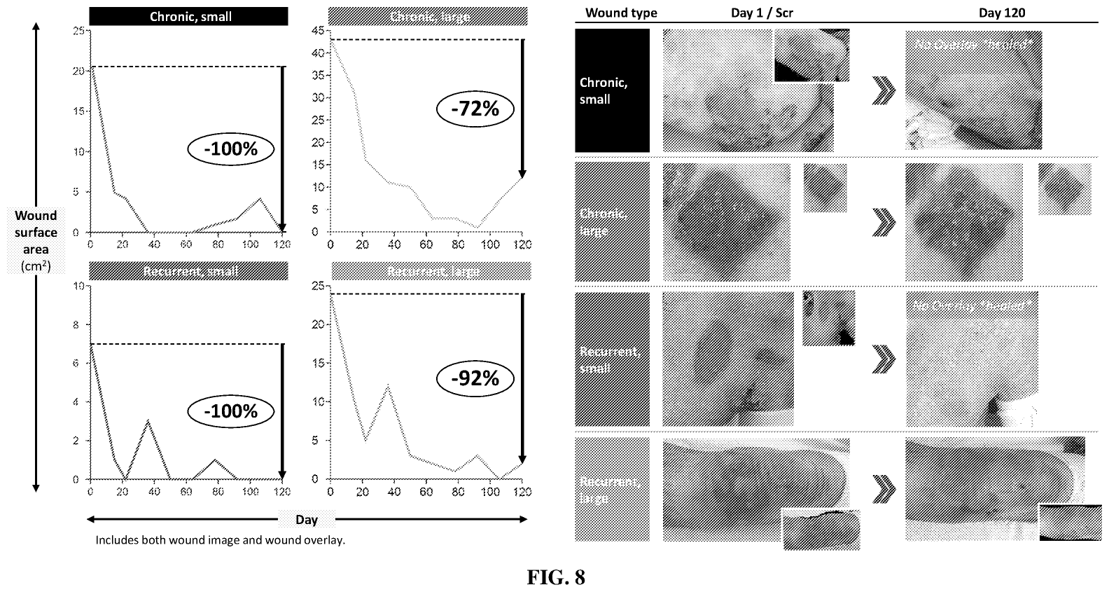

FIG. 8 shows example wound images demonstrating clear reduction in wound surface area post PTR-01 treatment across all types and sizes of wounds.

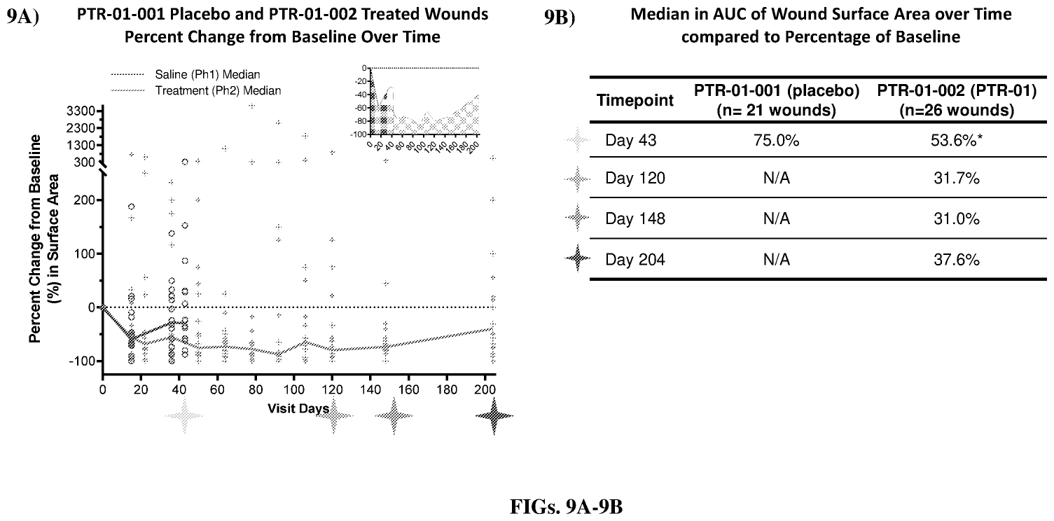

FIGS. 9A-9B show individual wound and median change from baseline in wound surface area. Using area under the curve (AUCi) analysis to examine wound size over time relative to baseline, there was a greater reduction at Day 43 in patients receiving PTR-01 than that observed in a historic Phase 1 PTR-01 study with patients receiving placebo (53.6% vs. 75%).

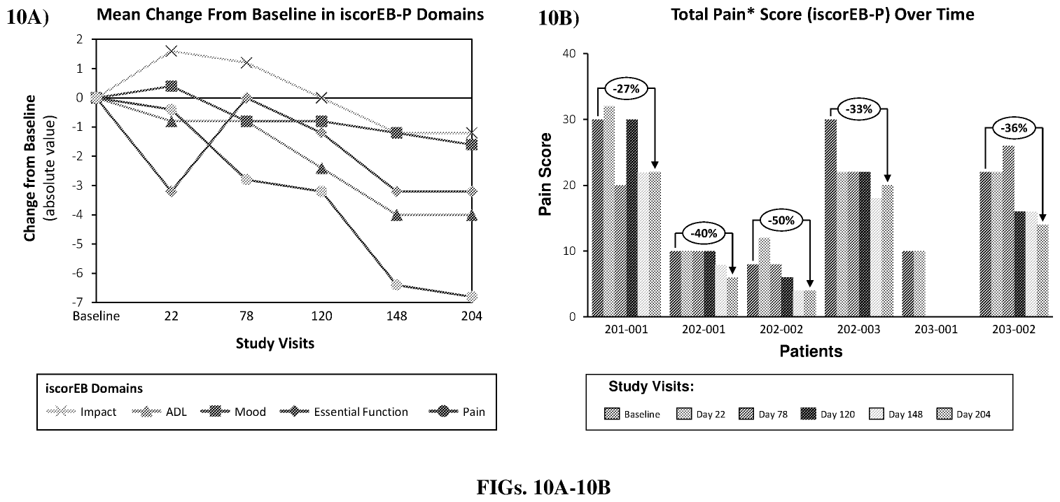

FIGS. 10A-10B show improvements in pain, disease impact, activities of daily living, mood, and essential functions measured by iscorEB-P by patients in the Phase 2 study. Marked mean and median reductions from baseline to Day 204 were observed in iscorEB scores for these metrics.

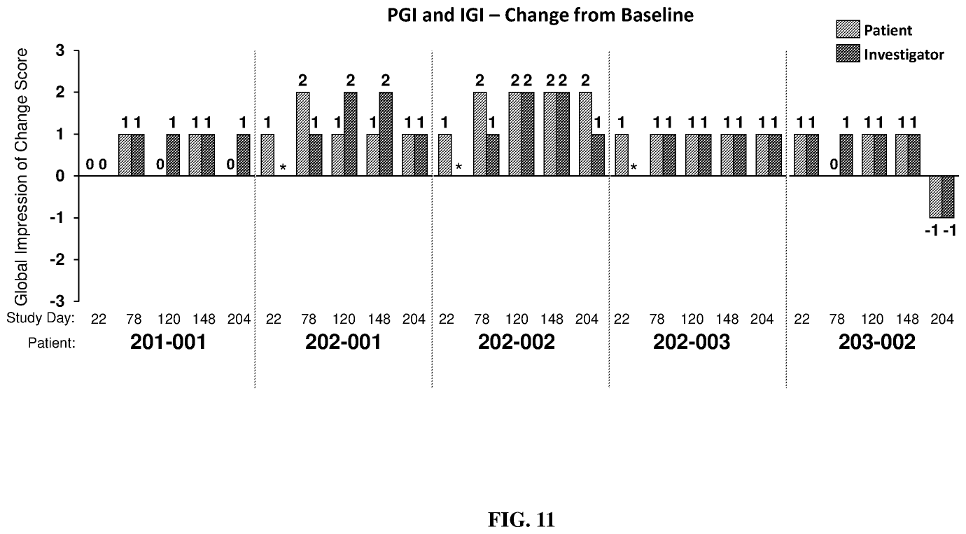

FIG. 11 shows Investigator and Patient Global Impression of Change (GIC) score by patient and time point. At Days 22, 78, 120, and 148, both IGIC and PGIC scores improved, with good correlation between investigator and patient assessments.

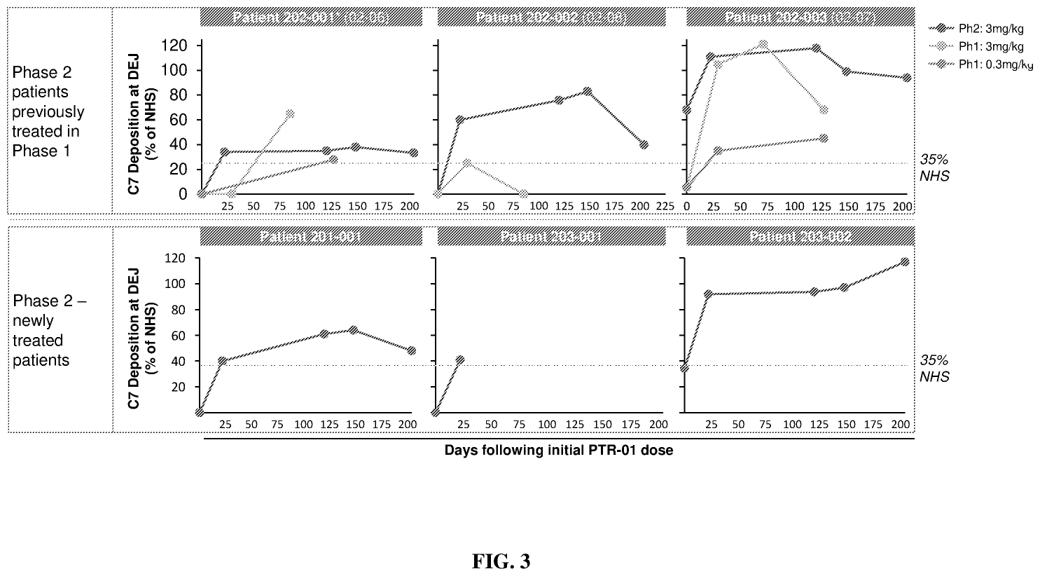

FIG. 3 shows immunofluorescence data showing deposition of C7 at the dermal-epidermal junction (DEJ) following administration of PTR-01. The upper panels show deposition for patients previously treated in the Phase 1 study described in Example 5, and the lower panels show deposition for patients not treated in the prior study. As shown in FIG. 3, deposition of C7 at the DEJ in patients who received PTR-01 at 0.3 mg/kg, and 3 mg/kg was dose dependent deposition. Rapid deposition of C7 at the DEJ was observed during the loading phase, achieving levels projected to confer a therapeutic effect (35% of normal). These levels were maintained throughout treatment and for one month following treatment completion. All patients exhibited continued deposition of C7 in the DEJ compartment ranging from one to three months following their last dose of PTR-01 (e.g., Day 148 and Day 204 timepoints). These data support the possibility of monthly, or perhaps longer, maintenance dosing.

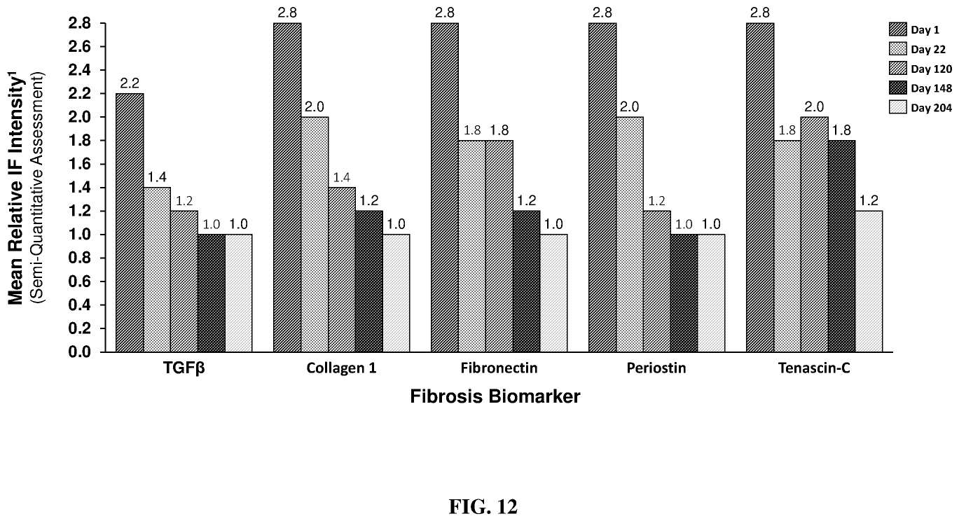

FIG. 12 shows reduction in skin pro-fibrotic biomarker staining with PTR-01 administration. Biomarkers of fibrosis decreased over the course of treatment from an elevated baseline, and remained reduced after treatment completion. Overall, PTR-01 was well tolerated when given once per week for 4 weeks and then every other week for 14 weeks. Table 10 summarizes TEAEs observed in the study. Twenty AEs were reported for 4 patients, all of which were resolved. No deaths, severe adverse events, or unexpected AEs were observed, and no AEs led to treatment discontinuation. All AEs were mild or moderate except a single AE of anemia, which was considered not related to the study drug. One patient had infusion reactions that responded to supportive care and resolved within hours. Three patients had detectable low-titer ADAs, observed at least once during the study. These observations were not associated with clinical or laboratory manifestations. One patient had high-titer ADAs. This patient had mild infusion reactions and eventually withdrew from the study due to lack of efficacy.

TABLE 10
TEAEs observed in the Phase 2 study

| | # of | |
|---|---|---|
| | patients | |
| TEAE | (events) | Grades |
| Any TEAE | 4 (20) | I, II, III |
| IARs/AESIs | 1 (10) | I |
| Infections | 1 (2) | I |
| Palpitations | 1 (1) | I |
| Dermatitis | 1 (1) | I |
| Other vitals/lab abnormalities | 2 (5) | I, II |
| Anemia | 1 (1) | III |

In summary, weekly infusions of PTR-01 at 3.0 mg/kg per week for 4 weeks followed by every other week (q.o.w.) infusions for 14 weeks were well-tolerated and resulted in rapid and sustained improvements in measures of wound healing including the proportion of patients with at least a 2 point improvement in the majority of wounds and the proportion of total patient wounds with ≥50% reduction in surface area; reduction in iscorEB symptom scores; deposition of rC7 at the DEJ; and reduction of pro-fibrotic biomarkers in the skin. Investigator and patient global assessments of change were in agreement and reflected improvement in overall disease. The results of this study support further investigation of PTR-01 administration for the treatment of DEB.

1. A method of treating dystrophic epidermolysis bullosa (DEB) in a subject in need thereof, comprising intravenously administering to the subject a recombinant human Col7 (rhCol7) substance according to a first dosing regimen and a second dosing regimen, wherein:
i) the first dosing regimen includes administering a first therapeutically effective amount of the substance at a first frequency; and
ii) the second dosing regimen includes administering a second therapeutically effective amount of the substance at a second frequency,
wherein the first frequency is greater than the second frequency, and wherein administration according to the first dosing regimen occurs prior to administration according to the second dosing regimen.

2. A method of ameliorating, alleviating, reducing, improving, delaying onset of, delaying progression of, eliminating, and/or curing a symptom or complication of dystrophic epidermolysis bullosa (DEB) in a subject in need thereof, comprising intravenously administering to the subject a recombinant human Col7 (rhCol7) substance according to a first dosing regimen and a second dosing regimen, wherein:
i) the first dosing regimen includes administering a first therapeutically effective amount of the substance at a first frequency; and
ii) the second dosing regimen includes administering a second therapeutically effective amount of the substance at a second frequency,
wherein the first frequency is greater than the second frequency, and wherein administration according to the first dosing regimen occurs prior to administration according to the second dosing regimen.

3. The method of claim 2, wherein the symptom or complication of DEB is skin wounds including blistering, itching, pain, skin erosion, scarring, skin fragility, gangrene, aplasia or hypoplasia of the skin, hypopigmentation of skin, milia, skin infections, cheilitis (e.g., inflammation of lips), dystrophic fingernails or toenails, abnormal dental enamel, pseudosyndactyly or camptodactyly of fingers or toes, finger or toe syndactyly, flexion contracture of toes, carious teeth, dysphagia (e.g., poor swallowing), furrowed or grooved tongue, esophageal stricture, laryngeal stenosis, failure to thrive, dilated cardiomyopathy, abnormal pulmonary interstitial morphology, acute constipation, hearing or visual impairment, eczema, glomerulopathy, immunologic hypersensitivity, nasolacrimal duct obstruction, anemia, chronic ear infections, corneal erosion, corneal abrasion and scarring, oral erosions, microstomia, osteopenia, ectropion, immunologic hypersensitivity, nephrotic syndrome, phimosis, renal insufficiency, urinary retention, ureteral stenosis, stroke, squamous cell carcinoma, or a combination thereof.

4. The method of claim 3, wherein the symptom or complication of DEB is skin wounds including blistering, skin erosion, scarring, skin fragility, aplasia or hypoplasia of the skin, cheilitis (e.g., inflammation of lips), dystrophic fingernails or toenails, pseudosyndactyly or camptodactyly of fingers or toes, finger or toe syndactyly, flexion contracture of toes, dysphagia (e.g., poor swallowing), esophageal stricture, laryngeal stenosis, or a combination thereof.

5. The method of claim 3, wherein the symptom or complication of DEB is skin wounds including blistering.

6. The method of any one of claims 2-5, wherein the method ameliorates, alleviates, reduces, improves, or eliminates one or more skin wounds.

7. The method of any one of claims 1-6, wherein the first dosing regimen lasts at least one week.

8. The method of any one of claims 1-7, wherein the first dosing regimen lasts at least two weeks.

9. The method of any one of claims 1-8, wherein the first dosing regimen lasts at least three weeks.

10. The method of any one of claims 1-9, wherein the first dosing regimen lasts at least four weeks.

11. The method of any one of claims 1-10, wherein the first frequency is about daily, about every two days, about every three days, about every four days, about every five days, about every six days, about every week, about every ten days, or about every two weeks.

12. The method of claim 11, wherein the first frequency is weekly.

13. The method of any one of claims 1-12, wherein the second dosing regimen lasts at least four weeks.

14. The method of any one of claims 1-13, wherein the second dosing regimen lasts at least six weeks.

15. The method of any one of claims 1-14, wherein the second dosing regimen lasts at least seven weeks.

16. The method of any one of claims 1-15, wherein the second dosing regimen lasts at least eight weeks.

17. The method of any one of claims 1-16, wherein the second dosing regimen lasts at least six months.

18. The method of any one of claims 1-17, wherein the second dosing regimen lasts at least one year.

19. The method of any one of claims 1-18, wherein the second dosing regimen comprises administering the rhCol7 substance to the subject over the subject's lifetime.

20. The method of any one of claims 1-19, wherein the second frequency is about every week, about every ten days, about every two weeks, about every three weeks, about every four weeks, or about every month.

21. The method of claim 20, wherein the second frequency is weekly.

22. The method of claim 20, wherein the second frequency is every other week.

23. The method of any one of claims 1-6, wherein the first dosing regimen lasts between one week and four weeks, and the second dosing regimen lasts at least six weeks.

24. The method of claim 23, wherein the first dosing regimen lasts one week.

25. The method of claim 23, wherein the first dosing regimen lasts two weeks.

26. The method of claim 23, wherein the first dosing regimen lasts three weeks.

27. The method of claim 23, wherein the first dosing regimen lasts four weeks.

28. The method of any one of claims 23-27, wherein the second dosing regimen lasts seven weeks.

29. The method of any one of claims 23-27, wherein the second dosing regimen lasts at least six months.

30. The method of any one of claims 23-27, wherein the second dosing regimen comprises administering the rhCol7 substance to the subject over the subject's lifetime.

31. The method of claim 23, wherein the first dosing regimen lasts four weeks, and the second dosing regimen lasts at least seven weeks.

32. The method of any one of claims 1-31, wherein the first effective amount is about 0.01 milligrams per kilogram of subject (mg/kg), about 0.03 mg/kg, about 0.05 mg/kg, about 0.1 mg/kg, about 0.2 mg/kg, about 0.3 mg/kg, about 0.4 mg/kg, about 0.5 mg/kg. about 0.6 mg/kg, about 0.7 mg/kg, about 0.8 mg/kg, about 0.9 mg/kg, about 1.0 mg/kg, about 1.1 mg/kg about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, about 10 mg/kg, about 15 mg/kg, or about 20 mg/kg.

33. The method of claim 32, wherein the first effective amount is about 0.1 mg/kg. 0.3 mg/kg. 0.5 mg/kg, 1.0 mg/kg, 1.5 mg/kg, 2.0 mg/kg, or 3.0 mg/kg.

34. The method of claim 33, wherein the first effective amount is about 0.3 mg/kg.

35. The method of claim 33, wherein the first effective amount is about 3.0 mg/kg.

36. The method of any one of claims 1-35, wherein the second effective amount is about 0.01 milligrams per kilogram of subject (mg/kg), about 0.03 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg. about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, about 10 mg/kg, about 15 mg/kg, or about 20 mg/kg.

37. The method of claim 36, wherein the second effective amount is about 0.1 mg/kg, 0.3 mg/kg, 0.5 mg/kg, 1.0 mg/kg, 1.5 mg/kg, 2.0 mg/kg, or 3.0 mg/kg.

38. The method of claim 37, wherein the second effective amount is about 0.3 mg/kg.

39. The method of claim 37, wherein the second effective amount is about 3.0 mg/kg.

40. The method of any one of claims 1-31, wherein the first effective amount is the same as the second effective amount.

41. The method of claim 40, wherein the first effective amount and the second effective amount are about 0.01 milligrams per kilogram of subject (mg/kg), about 0.03 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg. about 7.5 mg/kg, about 8.0 mg/kg, about 8.5 mg/kg, about 9.0 mg/kg, about 9.5 mg/kg. about 10 mg/kg, about 15 mg/kg, or about 20 mg/kg.

42. The method of claim 41, wherein the first effective amount and the second effective amount are about 0.1 mg/kg. 0.3 mg/kg, 0.5 mg/kg, 1.0 mg/kg, 1.5 mg/kg, 2.0 mg/kg, or 3.0 mg/kg.

43. The method of claim 42, wherein the first effective amount and the second effective amount are about 0.3 mg/kg.

44. The method of claim 42, wherein the first effective amount and the second effective amount are about 3.0 mg/kg.

45. The method of any one of claims 1-6, wherein the first dosing regimen lasts at least one week, the first frequency is weekly, and the first effective amount is about 0.1 mg/kg, 0.3 mg/kg, 0.5 mg/kg. 1.0 mg/kg, 1.5 mg/kg, 2.0 mg/kg, or 3.0 mg/kg.

46. The method of claim 45, wherein the first dosing regimen lasts four weeks.

47. The method of claim 45 or 46, wherein the first effective amount is about 0.3 mg/kg.

48. The method of claim 45 or 46, wherein the first effective amount is about 3.0 mg/kg.

49. The method of any one of claims 45-48, wherein the second dosing regimen lasts at least four weeks, the second frequency is every other week, and the second effective amount is about 0.1 mg/kg, 0.3 mg/kg, 0.5 mg/kg, 1.0 mg/kg, 1.5 mg/kg, 2.0 mg/kg, or 3.0 mg/kg.

50. The method of claim 49, wherein the second dosing regimen lasts at least seven weeks.

51. The method of claim 49 or 50, wherein the second effective amount is about 0.3 mg/kg.

52. The method of claim 49 or 50, wherein the second effective amount is about 3.0 mg/kg.

53. The method of any one of claims 1-52, wherein the intravenous administration is by intravenous infusion, or slow push injection.

54. The method of any one of claims 1-53, wherein the subject has recessive dystrophic epidermolysis bullosa (RDEB).

55. The method of any one of claims 1-53, wherein the subject has dominant dystrophic epidermolysis bullosa (DDEB).

56. The method of any one of claims 1-55, wherein the subject has a mutation in the COL7A1 gene that is consistent with a recessive inheritance pattern

57. The method of any one of claims 1-56, wherein the subject is a human.

58. The method of any one of claims 1-57, wherein the subject is under 18 years of age.

59. The method of claim 58, wherein the subject is at least 6 years of age.

60. The method of claim 58, wherein the subject is at least 2 years of age.

61. The method of claim 58, wherein the subject is less than 2 years of age.

62. The method of any one of claims 1-57, wherein the subject is at least 18 years of age.

63. The method of any one of claims 1-62, wherein the rhCol7 substance is included in a pharmaceutical composition comprising 1.2 milligrams per milliliter (mg/mL) rhCol7 substance, 10 millimolar (mM) sodium phosphate, 5 mM sodium citrate, 100 mM L-arginine, 1.7% sucrose (weight/volume), 70 mM sodium chloride, and 0.05% (volume/volume) polysorbate 20 (pH 7.2).

64. The method of any one of claims 1-63, wherein the second dosing regimen duration, second frequency, and/or second effective amount is determined at least in part based on an assessment of one or more clinical parameters of the subject performed during or after the first dosing regimen.

65. The method of any one of claims 1-64, further comprising, between the first dosing regimen and the second dosing regimen, assessing one or more clinical parameters.

66. The method of any one of claims 1-65, further comprising, during the first dosing regimen, assessing one or more clinical parameters.

67. The method of any one of claims 1-66, further comprising, during the second dosing regimen, assessing one or more clinical parameters.

68. The method of any one of claims 64-67, wherein the one or more clinical parameters are selected from wound surface area, wound healing, time to chronic wound healing, and time in re-blistering.
